(19) United States
(12) Patent Application Publication
(10) Pub. No.: US 2026/0207334 A1
(43) Pub. Date: Jul. 23, 2026

(54) HEART VALVE REPAIR DEVICES AND DELIVERY DEVICES THEREFOR

(71) Applicant: Edwards Lifesciences Corporation, Irvine, CA (US)

(72) Inventors: Grant Matthew Stearns, Costa Mesa, CA (US); Lauren R. Freschauf, Mission Viejo, CA (US); Quang Ngoc Vu, Aliso Viejo, CA (US); Keval Rajesh Savla, Irvine, CA (US); Nicholas William Humphreys, Aliso Viejo, CA (US); Christina Marie Santini, Huntington Beach, CA (US)

(21) Appl. No.: 19/568,789

(22) Filed: Mar. 17, 2026

Related U.S. Application Data
(63) Continuation of application No. PCT/US2024/045582, filed on Sep. 6, 2024.
(60) Provisional application No. 63/583,359, filed on Sep. 18, 2023.

(51) Int. Cl. A61F 2/24

(52) U.S. Cl. CPC A61F 2/2463; A61F 2/246; A61F 2220/0091; A61F 2250/0004

(57) ABSTRACT

Devices are configured to be positioned within a native heart valve to impede regurgitation through the native heart valve. The devices can have adjustable width frame members or paddles. A width of one or more the adjustable width frame members or paddles can be adjusted by moving portions of the one or more adjustable width frame members through one or more guides. The one or more guides control a shape of the one or more adjustable width frame members as the one or more adjustable width frame members move between a narrow configuration and a wide configuration. The one or more adjustable width frame members or paddles can be connected to a distal end portion of the device and pulled into a proximal end portion of the device.

### RELATED APPLICATIONS

The present application is a continuation of Patent Cooperation Treaty Application No. PCT/US2024/045582, filed on Sep. 6, 2024, titled “Heart Valve Repair Devices and Delivery Devices Therefor”, which claims the benefit of U.S. Provisional Application No. 63/583,359 filed on Sep. 18, 2023, titled “Heart Valve Repair Devices and Delivery Devices Therefor”, which are both incorporated herein by reference in their entireties.

### BACKGROUND

The native heart valves (i.e., the aortic, pulmonary, tricuspid, and mitral valves) serve critical functions in assuring the forward flow of an adequate supply of blood through the cardiovascular system. These heart valves may be damaged, and thus rendered less effective, for example, by congenital malformations, inflammatory processes, infectious conditions, disease, etc. Such damage to the valves may result in serious cardiovascular compromise or death. Damaged valves can be surgically repaired or replaced during open heart surgery. However, open heart surgeries are highly invasive, and complications may occur. Transvascular techniques can be used to introduce and implant devices to treat a heart in a manner that is much less invasive than open heart surgery. As one example, a transvascular technique useable for accessing the native mitral and aortic valves is the trans-septal technique. The trans-septal technique comprises advancing a catheter into the right atrium (e.g., inserting a catheter into the right femoral vein, up the inferior vena cava and into the right atrium). The septum is then punctured, and the catheter passed into the left atrium. A similar transvascular technique can be used to implant a device within the tricuspid valve that begins similarly to the trans-septal technique but stops short of puncturing the septum and instead turns the delivery catheter toward the tricuspid valve in the right atrium.

A healthy heart has a generally conical shape that tapers to a lower apex. The heart is four-chambered and comprises the left atrium, right atrium, left ventricle, and right ventricle. The left and right sides of the heart are separated by a wall generally referred to as the septum. The native mitral valve of the human heart connects the left atrium to the left ventricle. The mitral valve has a very different anatomy than other native heart valves. The mitral valve includes an annulus portion, which is an annular portion of the native valve tissue surrounding the mitral valve orifice, and a pair of cusps, or leaflets, extending downward from the annulus into the left ventricle. The mitral valve annulus may form a “D”-shaped, oval, or otherwise out-of-round cross-sectional shape having major and minor axes. The anterior leaflet may be larger than the posterior leaflet, forming a generally “C”-shaped boundary between the abutting sides of the leaflets when they are closed together.

When operating properly, the anterior leaflet and the posterior leaflet function together as a one-way valve to allow blood to flow only from the left atrium to the left ventricle. The left atrium receives oxygenated blood from the pulmonary veins. When the muscles of the left atrium contract and the left ventricle dilates (also referred to as “ventricular diastole” or “diastole”), the oxygenated blood that is collected in the left atrium flows into the left ventricle. When the muscles of the left atrium relax and the muscles of the left ventricle contract (also referred to as “ventricular systole” or “systole”), the increased blood pressure in the left ventricle urges the sides of the two leaflets together, thereby closing the one-way mitral valve so that blood cannot flow back to the left atrium and is instead expelled out of the left ventricle through the aortic valve. To prevent the two leaflets from prolapsing under pressure and folding back through the mitral annulus toward the left atrium, a plurality of fibrous cords called chordae tendineae tether the leaflets to papillary muscles in the left ventricle.

Valvular regurgitation involves the valve improperly allowing some blood to flow in the wrong direction through the valve. For example, mitral regurgitation occurs when the native mitral valve fails to close properly and blood flows into the left atrium from the left ventricle during the systolic phase of heart contraction. Mitral regurgitation is one of the most common forms of valvular heart disease. Mitral regurgitation may have many different causes, such as leaflet prolapse, dysfunctional papillary muscles, stretching of the mitral valve annulus resulting from dilation of the left ventricle, more than one of these, etc. Mitral regurgitation at a central portion of the leaflets can be referred to as central jet mitral regurgitation and mitral regurgitation nearer to one commissure (i.e., location where the leaflets meet) of the leaflets can be referred to as eccentric jet mitral regurgitation. Central jet regurgitation occurs when the edges of the leaflets do not meet in the middle and thus the valve does not close, and regurgitation is present. Tricuspid regurgitation may be similar, but on the right side of the heart.

### SUMMARY

This summary is meant to provide some examples and is not intended to limit of the scope of the disclosed subject matter in any way. For example, any feature included in an example of this summary is not required by the claims, unless the claims explicitly recite the feature. Also, the features, components, steps, concepts, etc. described in examples in this summary and elsewhere in this disclosure can be combined in a variety of ways. Various features and steps as described elsewhere in this disclosure can be included in the examples summarized here.

Devices for repairing and/or treating a native valve of a patient are disclosed. The devices can be valve repair devices, implantable devices, valve treatment devices, implants, etc. While the devices may be described and/or depicted as implantable devices in some examples herein, similar configurations can be used on other devices, e.g., valve repair devices, treatment devices, etc., that are not necessarily implanted and may be removed after treatment.

In some implementations, there is provided a device (e.g., a treatment device, a repair device, a valve repair device, a valve treatment device, a prosthetic device, an implantable device, an implant, etc.) that is configured to be positioned within a native heart valve to allow the native heart valve to form a more effective seal.

In some implementations, a device or implant (e.g., a treatment device, a repair device, a valve repair device, a valve treatment device, a prosthetic device, an implantable device, an implant, etc.) includes an anchor portion. In some implementations, the anchor portion includes one or more anchors. In some implementations, each anchor includes one or more paddles that are each moveable between an open position and a closed position.

In some implementations, a device useable for treating and/or repairing a native valve of a heart (e.g., a treatment device, a repair device, a valve repair device, a valve treatment device, a prosthetic device, an implantable device, an implant, etc.) includes one or more paddles (e.g., at least one paddle), one or more adjustable width frame members (e.g., at least one adjustable width frame member), and/or one or more guides (e.g., at least one guide). In some implementations, the one or more paddles are movable between an open position and a closed position.

In some implementations, the one or more paddles are configured to attach the device to the native valve. In some implementations, the one or more adjustable width frame members are coupled to the one or more paddles such that the one or more adjustable width frame members open and close with the one or more paddles.

In some implementations, the one or more adjustable width frame members extend through the one or more guides. In some implementations, the one or more guides control a shape of the one or more adjustable width frame members as the adjustable width frame members move between a narrow configuration and a wide configuration.

In some implementations, the one or more paddles comprise a pair of paddles, the one or more adjustable width frame members comprise a pair of adjustable width frame members, and/or the one or more guides comprise a pair of guides. In some implementations, the pair of paddles are movable between an open position and a closed position. In some implementations, the pair of paddles are configured to attach the device to the native valve. In some implementations, the pair of adjustable width frame members are coupled to the pair of paddles such that the pair of adjustable width frame members open and close with the pair of paddles. In some implementations, the pair of adjustable width frame members extend through the pair of guides. In some implementations, the pair of guides control a shape of the pair of adjustable width frame members as the adjustable width frame members move between a narrow configuration and a wide configuration.

In some implementations, the device includes a connector coupled to the one or more adjustable width frame members. In some implementations, the connector is configured to move the one or more adjustable width frame members between the narrow configuration and the wide configuration.

In some implementations, the connector includes attachment portions that connect to the adjustable width frame members.

In some implementations, one or more inner frame members (e.g., one inner frame members, a pair of inner frame members, three inner frame members, etc.) are coupled to the one or more paddles (e.g., to a pair of paddles). In some implementations, the one or more inner frame members are configured to bias the one or more paddles to a closed position.

In some implementations, each of the adjustable width frame members have an anterior portion extending from the medial side of the device to the lateral side of the device and a posterior portion extending from the medial side of the device to the lateral side of the device opposite the anterior portion.

In some implementations, the anterior portion and the posterior portion of each adjustable width frame member attach to one, or multiple, of the inner frame portion(s) and the one or more paddles at a proximal portion of the device.

In some implementations, each of the adjustable width frame members comprise a shape-memory alloy.

In some implementations, the one or more adjustable width frame members are symmetric.

In some implementations, each guide of the one or more guides comprises a sling.

In some implementations, each guide of the one or more guides comprises one or more loops (e.g., a pair of loops, etc.).

In some implementations, the one or more adjustable width frame members are attached to a proximal portion of the device.

In some implementations, the one or more adjustable width frame members are attached to a distal portion of the device.

In some implementations, the one or more adjustable width frame members are attached to a proximal portion of the device and the connector is pulled into a distal portion of the device to narrow the one or more adjustable width frame members.

In some implementations, the one or more adjustable width frame members are attached to a distal portion of the device and the connector is pulled into a proximal portion of the device to narrow the one or more adjustable width frame members.

In some implementations, a system (e.g., a treatment system, a repair system, a valve repair system, a valve treatment system, etc.) comprises a treatment and/or repair device (e.g., a treatment device, a repair device, a valve repair device, a valve treatment device, a prosthetic device, an implantable device, an implant, etc.) and a delivery device.

In some implementations, the device includes one or more paddles (e.g., a paddle, at least one paddle, a pair of paddles, three paddles, etc.), one or more adjustable width frame members (e.g., an adjustable width frame member, at least one adjustable width frame member, a pair of adjustable width frame members, three adjustable width frame members, etc.), and one or more guides (e.g., a guide, at least one guide, a pair of guides, three guides, etc.).

In some implementations, the one or more paddles are movable between an open position and a closed position. In some implementations, the one or more paddles are configured to attach the device to the native valve.

In some implementations, the one or more adjustable width frame members are coupled to the one or more paddles such that the one or more adjustable width frame members open and close with the one or more paddles.

In some implementations, the one or more adjustable width frame members extend through the one or more guides. In some implementations, the one or more guides control a shape of the one or more adjustable width frame members as the adjustable width frame members move between a narrow configuration and a wide configuration.

In some implementations, the delivery device includes an opening and closing element and a width control element. In some implementations, the opening and closing control element is coupled to the one or more paddles. In some implementations, the opening and closing element is configured to move the one or more paddles between an open position and a closed position.

In some implementations, the width control element is coupled to the one or more adjustable width frame members and is configured to move the one or more adjustable width frame members move between the narrow configuration and the wide configuration.

In some implementations, a device (e.g., a treatment device, a repair device, a valve repair device, a valve treatment device, a prosthetic device, an implantable device, an implant, etc.) includes one or more paddles (e.g., a paddle, at least one paddle, a pair of paddles, three paddles, etc.) and one or more adjustable width frame members (e.g., an adjustable width frame member, at least one adjustable width frame member, a pair of adjustable width frame members, three adjustable width frame members, etc.).

In some implementations, the one or more paddles are movable between an open position and a closed position. In some implementations, the one or more paddles are configured to attach the device to the native valve.

In some implementations, the one or more adjustable width frame members are coupled to the one or more paddles such that the one or more adjustable width frame members open and close with the one or more paddles. In some implementations, the one or more adjustable width frame members are attached to a distal portion of the device.

In some implementations, the one or more adjustable width frame members are attached directly to a distal portion of a main body of the device.

In some implementations, a spacer is disposed over the main body of the device.

In some implementations, the one or more adjustable width frame members are attached directly to a cap of the device.

In some implementations, movement of the cap moves the one or more paddles between the open position and the closed position.

In some implementations, a connector is coupled to the one or more adjustable width frame members. In some implementations, the connector is configured to move the one or more adjustable width frame members between the narrow configuration and the wide configuration.

In some implementations, the connector is pulled into a proximal portion of the device to narrow the one or more adjustable width frame members.

In some implementations, the connector is pulled into a collar of the device.

In some implementations, the connector includes attachment portions that connect to the adjustable width frame members.

In some implementations, one or more inner frame members (e.g., an inner frame member, at least one inner frame member, a pair of inner frame members, three frame members, etc.) are coupled to the one or more paddles. In some implementations, the one or more inner frame members are configured to bias the one or more paddles to a closed position.

In some implementations, each of the adjustable width frame members comprise a shape-memory alloy.

In some implementations, the one or more adjustable width frame members are symmetric.

In some implementations, a system (e.g., a treatment system, a repair system, a valve repair system, a valve treatment system, etc.) includes a treatment and/or repair device (e.g., a treatment device, a repair device, a valve repair device, a valve treatment device, a prosthetic device, an implantable device, an implant, etc.) and a delivery device.

In some implementations, the device includes one or more paddles (e.g., a paddle, at least one paddle, a pair of paddles, three paddles, etc.) and one or more adjustable width frame members (e.g., an adjustable width frame member, at least one adjustable width frame member, a pair of adjustable width frame members, three adjustable width frame members, etc.). In some implementations, the one or more paddles are movable between an open position and a closed position. In some implementations, the one or more paddles are configured to attach the device to the native valve.

In some implementations, the one or more adjustable width frame members are coupled to the one or more paddles such that the one or more adjustable width frame members open and close with the one or more paddles. In some implementations, the one or more adjustable width frame members are attached to a distal portion of the device.

In some implementations, the delivery device includes an opening and closing element and a width control element. In some implementations, the opening and closing control element is coupled to the one or more paddles. In some implementations, the opening and closing element is configured to move the one or more paddles between an open position and a closed position.

In some implementations, the width control element is coupled to the one or more adjustable width frame members and is configured to move the one or more adjustable width frame members move between the narrow configuration and the wide configuration.

In some implementations, the one or more paddles comprise a pair of paddles and the one or more adjustable width frame members comprise a pair of adjustable width frame members. In some implementations, the pair of paddles are movable between an open position and a closed position. In some implementations, the pair of paddles are configured to attach the device to the native valve. In some implementations, the pair of adjustable width frame members are coupled to the pair of paddles such that the pair of adjustable width frame members open and close with the pair of paddles. In some implementations, the pair of adjustable width frame members are attached to a distal portion of the device. In some implementations, the opening and closing control element is coupled to the pair of paddles. In some implementations, the opening and closing element is configured to move the pair of paddles between an open position and a closed position. In some implementations, the width control element is coupled to the pair of adjustable width frame members and is configured to move the pair of adjustable width frame members move between the narrow configuration and the wide configuration.

In some implementations, a method of repairing a native valve of a heart includes moving one or more paddles (e.g., a paddle, at least one paddle, a pair of paddles, three paddles, etc.) from a closed position to an open position. In some implementations, the one or more paddles are moved from open position to the closed position to attach the device to the native valve. In some implementations, a width of one or more adjustable width frame members (e.g., an adjustable width frame member, at least one adjustable width frame member, a pair of adjustable width frame members, three adjustable width frame members, etc.) is adjusted by moving portions of the one or more adjustable width frame members through one or more guides (e.g., a guide, at least one guide, a pair of guides, three guides, etc.).

In some implementations, the one or more guides control a shape of the one or more adjustable width frame members as the one or more adjustable width frame members move between the narrow configuration and the wide configuration.

In some implementations, a method of repairing a native valve of a heart includes moving one or more paddles (e.g., a paddle, at least one paddle, a pair of paddles, three paddles, etc.) from a closed position to an open position. In some implementations, the one or more paddles are moved from the open position to the closed position to attach the device to the native valve. In some implementations, one or more adjustable width frame members (e.g., an adjustable width frame member, at least one adjustable width frame member, a pair of adjustable width frame members, three adjustable width frame members, etc.) are narrowed by pulling the one or more adjustable width frame members proximally.

In some implementations, the one or more adjustable width frame members are attached directly to a distal portion of a main body of the device.

In some implementations, the one or more adjustable width frame members are pulled proximally by pulling a connector into a proximal portion of the device.

In some implementations, the one or more adjustable width frame members are widened by pushing the one or more adjustable width frame members distally.

In some implementations, the one or more adjustable width frame members are pushed distally by pushing a connector out of a proximal portion of the device.

In some implementations, apparatuses, systems (e.g., a treatment system, a repair system, a valve repair system, a valve treatment system, etc.), and/or methods described herein relate to a device useable for treating and/or repairing a native valve of a heart (e.g., a treatment device, a repair device, a valve repair device, a valve treatment device, a prosthetic device, an implantable device, an implant, etc.). The device can include a base. In some implementations, the device can include a spacer. In some implementations, the device can include a central body (e.g., a center support member, receiver, or bushing) having a proximal end portion, a distal end portion, and an inner space. In some implementations, the device can include a cap connected to the distal end portion of the central body. In some implementations, the device can include one or more paddles (e.g., a paddle, at least one paddle, a pair of paddles, three paddles, etc.). The one or more paddles can be movable between an open position and a closed position. The one or more paddles can be configured to attach the device to the native valve. In some implementations, the device can include one or more adjustable width frame members (e.g., an adjustable width frame member, at least one adjustable width frame member, a pair of adjustable width frame members, three adjustable width frame members, etc.) coupled to the one or more paddles such that the one or more adjustable width frame members open and close with the one or more paddles. In some implementations, the one or more adjustable width frame members include an outer frame portion extending from the distal end portion of the central body and extending from the inner space of the central body.

In some implementations, apparatuses, systems, and/or methods described herein relate to a device wherein the one or more adjustable width frame members (e.g., an adjustable width frame member, at least one adjustable width frame member, a pair of adjustable width frame members, three adjustable width frame members, etc.) further include an inner frame portion connected to the outer frame portion and having a location in the inner space of the central body (e.g., base, spacer, center support member, receiver, or bushing).

In some implementations, apparatuses, systems, and/or methods described herein relate to a device wherein the one or more adjustable width frame members (e.g., an adjustable width frame member, at least one adjustable width frame member, a pair of adjustable width frame members, three adjustable width frame members, etc.) include an expanded state having the outer frame portion in an extended position relative to the distal end portion of the central body (e.g., base, spacer, center support member, receiver, or bushing).

In some implementations, apparatuses, systems, and/or methods described herein relate to a device wherein the one or more adjustable width frame members (e.g., an adjustable width frame member, at least one adjustable width frame member, a pair of adjustable width frame members, three adjustable width frame members, etc.) include a narrowed state having the outer frame member portion in a retracted position relative to the distal end portion of the central body (e.g., base, spacer, center support member, receiver, or bushing).

In some implementations, apparatuses, systems, and/or methods described herein relate to a device wherein the outer frame portion of the one or more adjustable width frame members (e.g., an adjustable width frame member, at least one adjustable width frame member, a pair of adjustable width frame members, three adjustable width frame members, etc.) include an arcuate shape.

In some implementations, apparatuses, systems, and/or methods described herein relate to a device further including a connector extending through the inner space of the central body (e.g., base, spacer, center support member, receiver, or bushing) and connected to the one or more adjustable width frame members (e.g., an adjustable width frame member, at least one adjustable width frame member, a pair of adjustable width frame members, three adjustable width frame members, etc.).

In some implementations, apparatuses, systems, and/or methods described herein relate to a device wherein the outer frame portion of the one or more adjustable width frame members (e.g., an adjustable width frame member, at least one adjustable width frame member, a pair of adjustable width frame members, three adjustable width frame members, etc.) is configured to extend from and retract into the distal end portion of the central body (e.g., base, spacer, center support member, receiver, or bushing).

In some implementations, apparatuses, systems, and/or methods described herein relate to a device wherein the outer frame portion arcuately extends from the inner space of the central body (e.g., base, spacer, center support member, receiver, or bushing) and out from the distal end portion of the central body (e.g., base, spacer, center support member, receiver, or bushing).

In some implementations, apparatuses, systems, and/or methods described herein relate to a device wherein the outer frame portion is configured to open the one or more adjustable width frame members (e.g., an adjustable width frame member, at least one adjustable width frame member, a pair of adjustable width frame members, three adjustable width frame members, etc.) when the outer frame portion is retracted into the distal end portion of the central body (e.g., base, spacer, center support member, receiver, or bushing).

In some implementations, apparatuses, systems, and/or methods described herein relate to a device wherein the outer frame portion is configured to close the one or more adjustable width frame members (e.g., an adjustable width frame member, at least one adjustable width frame member, a pair of adjustable width frame members, three adjustable width frame members, etc.) when the outer frame portion is extended from the distal end portion of the central body (e.g., base, spacer, center support member, receiver, or bushing).

In some implementations, apparatuses, systems (e.g., a treatment system, a repair system, a valve repair system, a valve treatment system, etc.), and/or methods described herein relate to a device (e.g., a treatment device, a repair device, a valve repair device, a valve treatment device, a prosthetic device, an implantable device, an implant, etc.) useable for treating and/or repairing a native valve of a heart. The device can include one or more paddles (e.g., a paddle, at least one paddle, a pair of paddles, three paddles, etc.) movable between an open position and a closed position. The one or more paddles can be configured to attach the device to the native valve. In some implementation, the one or more paddles include an inner paddle portion and an outer paddle portion. In some implementations, the device can include one or more adjustable width frame members (e.g., an adjustable width frame member, at least one adjustable width frame member, a pair of adjustable width frame members, three adjustable width frame members, etc.) coupled to the one or more paddles such that the one or more adjustable width frame members open and close with the one or more paddles. In some implementations a paddle of the one or more paddles includes at least one guide portion (e.g., a guide portion, at least one guide portion, a pair of guide portions, three guide portions, etc.). In some implementations. In some implementations, the outer paddle portion includes at least one guide portion. In some implementations, portions of the one or more adjustable width frame members are positioned inside the at least one guide portion and can move within the at least one guide portion.

In some implementations, apparatuses, systems, and/or methods described herein relate to a device wherein the at least one guide portion (e.g., a guide portion, at least one guide portion, a pair of guide portions, three guide portions, etc.) includes at least one cut-out.

In some implementations, apparatuses, systems, and/or methods described herein relate to a device wherein the at least one guide portion (e.g., a guide portion, at least one guide portion, a pair of guide portions, three guide portions, etc.) includes at least one recess (e.g., a recess, at least one recess, a pair of recesses, three recesses, etc.).

In some implementations, apparatuses, systems, and/or methods described herein relate to a device wherein the at least one guide portion (e.g., a guide portion, at least one guide portion, a pair of guide portions, three guide portions, etc.) includes a closed shape having an inner open space.

In some implementations, apparatuses, systems, and/or methods described herein relate to a device wherein the at least one guide portion (e.g., a guide portion, at least one guide portion, a pair of guide portions, three guide portions, etc.) includes at least one curved section (e.g., a curved section, at least one curved section, a pair of curved sections, three curved sections, etc.).

In some implementations, apparatuses, systems, and/or methods described herein relate to a device wherein the at least one guide portion (e.g., a guide portion, at least one guide portion, a pair of guide portions, three guide portions, etc.) includes at least one projecting tab (e.g., a projecting tab, at least one projecting tab, a pair of projecting tabs, three projecting tabs, etc.).

In some implementations, apparatuses, systems, and/or methods described herein relate to a device wherein the a paddle portion of the one or more paddle portions includes a body portion having a distended shape section. In some implementations, an outer paddle portion of the paddle portion can include a body portion having a distended shape section.

In some implementations, apparatuses, systems, and/or methods described herein relate to a device wherein the distended shape section includes a first and second side portions, and wherein the first and second side portions each include the at least one guide (e.g., a guide, at least one guide, a pair of guides, three guides, etc.).

In some implementations, apparatuses, systems, and/or methods described herein relate to a device wherein the body portion includes first and second distended sections and the at least one guide between (e.g., a guide, at least one guide, a pair of guides, three guides, etc.) the first and second distended sections.

In some implementations, apparatuses, systems, and/or methods (e.g., a treatment apparatus, system, and/or method, a repair apparatus, system, and/or method, a valve repair apparatus, system, and/or method, a valve treatment apparatus, system, and/or method, etc.) described herein relate to a device (e.g., a treatment device, a repair device, a valve repair device, a valve treatment device, a prosthetic device, an implantable device, an implant, etc.) useable for treating and/or repairing a native valve of a heart. The device can include one or more paddles (e.g., a paddle, at least one paddle, a pair of paddles, three paddles, etc.) movable between an open position and a closed position. In some implementations the one or more paddles are configured to attach the device to the native valve. The device can include one or more adjustable width frame members (e.g., an adjustable width frame member, at least one adjustable width frame member, a pair of adjustable width frame members, three adjustable width frame members, etc.) coupled to the one or more paddles such that the one or more adjustable width frame members open and close with the one or more paddles. In some implementations, the one or more adjustable width frame members include a connection to the one or more paddles that includes a pivot joint.

In some implementations, apparatuses, systems, and/or methods described herein relate to a device wherein the pivot joint includes a hinge joint. In some implementations, apparatuses, systems, and/or methods described herein relate to a device wherein the pivot joint includes a rotational joint. In some implementations, apparatuses, systems, and/or methods described herein relate to a device wherein the pivot joint includes a flex joint.

In some implementations, apparatuses, systems, and/or methods described herein relate to a device wherein the one or more paddles (e.g., a paddle, at least one paddle, a pair of paddles, three paddles, etc.) include inner and outer paddle portions and the pivot joint includes a location on the outer paddle portions.

In some implementations, apparatuses, systems, and/or methods described herein relate to a device wherein the pivot joint includes a pin portion extending from the one or more paddles (e.g., a paddle, at least one paddle, a pair of paddles, three paddles, etc.).

In some implementations, apparatuses, systems, and/or methods described herein relate to a device wherein the adjustable width frame members include a pin ring portion.

In some implementations, apparatuses, systems, and/or methods described herein relate to a device further including at least one activation connector connected to the adjustable width frame members.

In some implementations, apparatuses, systems, and/or methods described herein relate to a device wherein the one or more paddles include at least one guide portion (e.g., a guide portion, at least one guide portion, a pair of guide portions, three guide portions, etc.) for restricting movement of the adjustable width frame members.

In some implementations, apparatuses, systems, and/or methods described herein relate to a device wherein the adjustable width frame members include portions that rotate or flex about the pivot joint.

In some implementations, apparatuses, systems, and/or methods described herein relate to a device further including a guide portion connected to the one or more paddles, wherein the guide portion includes a body having outer walls and an inner passageway. In some implementations, the inner passageway is configured to receive portions of the adjustable width frame members.

In some implementations, apparatuses, systems, and/or methods described herein relate to a device wherein the adjustable width frame members include at least one strut portion (e.g., a strut portion, at least one strut portion, a pair of strut portions, three strut portions, etc.) connected to the pivot joint and at least one tab portion (e.g., a tab portion, at least one tab portion, a pair of tab portions, three tab portions, etc.) connected to the pivot joint and wherein movement of tab portion causes pivoting of the strut portion about the pivot joint.

In some implementations, apparatuses, systems, and/or methods described herein relate to a device wherein the adjustable width frame members include at least one strut portion (e.g., a strut portion, at least one strut portion, a pair of strut portions, three strut portions, etc.) connected to the pivot joint and at least one tab portion (e.g., a tab portion, at least one tab portion, a pair of tab portions, three tab portions, etc.) connected to the pivot joint and wherein movement of tab portion causes flexure of the strut portion relative to the pivot joint.

In some implementations, apparatuses, systems, and/or methods described herein relate to a device wherein the adjustable width frame members include first and second strut portions connected together and wherein the first strut portion is configured to pivot about the pivot joint when the second strut portion is moved.

In some implementations, apparatuses, systems, and/or methods (e.g., a treatment apparatus, system, and/or method, a repair apparatus, system, and/or method, a valve repair apparatus, system, and/or method, a valve treatment apparatus, system, and/or method, etc.) described herein relate to a device (e.g., a treatment device, a repair device, a valve repair device, a valve treatment device, a prosthetic device, an implantable device, an implant, etc.) useable for treating and/or repairing a native valve of a heart. The device can include a central body (e.g., base, spacer, center support member, receiver, or bushing) having a proximal end portion and a distal end portion. In some implementations, the device can include a cap connected to the distal end portion of the central body and have a retaining portion. In some implementations, the device can include one or more paddles (e.g., a paddle, at least one paddle, a pair of paddles, three paddles, etc.) movable between an open position and a closed position. The one or more paddles can be configured to attach the device to the native valve. In some implementations, the device can include one or more adjustable width frame members (e.g., an adjustable width frame member, at least one adjustable width frame member, a pair of adjustable width frame members, three adjustable width frame members, etc.) coupled to the cap such that the one or more adjustable width frame members open and close with the one or more paddles. The one or more adjustable width frame members can include a connection to the cap that includes a pivot joint.

In some implementations, apparatuses, systems, and/or methods described herein relate to a device wherein the pivot joint includes a hinge joint. In some implementations, apparatuses, systems, and/or methods described herein relate to a device wherein the pivot joint includes a rotational joint. In some implementations, apparatuses, systems, and/or methods described herein relate to a device wherein the pivot joint includes a flex joint.

In some implementations, apparatuses, systems, and/or methods described herein relate to a device wherein the pivot joint includes a pin portion extending from the one or more paddles (e.g., a paddle, at least one paddle, a pair of paddles, three paddles, etc.).

In some implementations, apparatuses, systems, and/or methods described herein relate to a device wherein the adjustable width frame members include a pin ring portion.

In some implementations, apparatuses, systems, and/or methods described herein relate to a device wherein the cap includes a retaining portion connected to the distal end of the central body (e.g., base, spacer, center support member, receiver, or bushing) and the pivot joint includes a location on the retaining portion.

Any of the above method(s) and any methods of using the systems, assemblies, apparatuses, devices, etc. herein can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can optionally comprise computerized and/or physical representations.

Any of the above systems, assemblies, devices, apparatuses, components, etc. can be sterilized (e.g., with heat, radiation, ethylene oxide, hydrogen peroxide, etc.) to ensure they are safe for use with patients, and the methods herein can comprise (or additional methods comprise or consist of) sterilization of one or more systems, devices, apparatuses, components, etc. herein (e.g., with heat, radiation, ethylene oxide, hydrogen peroxide, etc.).

A further understanding of the nature and advantages of the present invention are set forth in the following description and claims, particularly when considered in conjunction with the accompanying drawings in which like parts bear like reference numerals.

### BRIEF DESCRIPTION OF THE DRAWINGS

To further clarify various aspects of examples in the present disclosure, a more particular description of certain examples and implementations will be made by reference to various aspects of the appended drawings. These drawings depict only example implementations of the present disclosure and are therefore not to be considered limiting of the scope of the disclosure. Moreover, while the figures can be drawn to scale for some examples, the figures are not necessarily drawn to scale for all examples. Examples and other features and advantages of the present disclosure will be described and explained with additional specificity and detail through the use of the accompanying drawings in which:

FIG. 1 illustrates a cutaway view of the human heart in a diastolic phase;

FIG. 2 illustrates a cutaway view of the human heart in a systolic phase;

FIG. 3 illustrates a cutaway view of the human heart in a systolic phase showing valve regurgitation;

FIG. 4 is the cutaway view of FIG. 3 annotated to illustrate a natural shape of mitral valve leaflets in the systolic phase;

FIG. 5 illustrates a healthy mitral valve with the leaflets closed as viewed from an atrial side of the mitral valve;

FIG. 6 illustrates a dysfunctional mitral valve with a visible gap between the leaflets as viewed from an atrial side of the mitral valve;

FIG. 7 illustrates a tricuspid valve viewed from an atrial side of the tricuspid valve;

FIGS. 8-14 show an example device or implant, in various stages of deployment;

FIG. 15 shows an example device that is similar to the device illustrated by FIGS. 8-14, but where the paddles are independently controllable;

FIGS. 16-21 show the example device of FIGS. 8-14 being delivered and deployed within a native valve;

FIG. 22 shows a perspective view of an example device in a closed position;

FIG. 23 shows a perspective view of an example device in a closed position;

FIG. 24 illustrates an example device with paddles in an open position;

FIG. 25A illustrates an example device with paddles in a closed position;

FIG. 25B illustrates a top view of an example device;

FIG. 26 illustrates a perspective view of an example device having paddles of adjustable widths;

FIG. 27 is a cross-section of the example device of FIG. 26 in which the device is bisected;

FIG. 28 is another cross-section of the example device of FIG. 26 in which the device is bisected along a plane perpendicular to the plane illustrated in FIG. 28;

FIG. 29 is a schematic illustration of an example catheter assembly coupled to an example device in which an actuation element is coupled to a paddle actuation control and to a driver head of the device;

FIG. 30 is an illustration of the assembly of FIG. 29 with the example device rotated 90 degrees to show the paddle width adjustment element coupled to an inner end of the connector of the device and coupled to a paddle width control;

FIG. 31 illustrates a perspective view of an example device having paddles of adjustable widths;

FIG. 32 illustrates a front view of the device of FIG. 31;

FIG. 33 illustrates a side view of the device of FIG. 31;

FIG. 34 illustrates a front view of an example device having paddles of adjustable widths;

FIG. 35 illustrates a perspective view of an example paddle frame useable as part of a device;

FIG. 36 illustrates a side view of the paddle frame of FIG. 34;

FIG. 37 illustrates a front view of the paddle frame of FIG. 34;

FIG. 38 illustrates a side view of an example device having paddles of adjustable widths with the paddles in a wide configuration;

FIG. 39 illustrates a front view of the device of FIG. 38;

FIG. 40 illustrates a side view of the device of FIG. 38 with the paddles in a narrow configuration;

FIG. 41 illustrates a front view of the device of FIG. 40;

FIG. 42 illustrates a perspective view of an example device having paddles of adjustable widths;

FIG. 43 illustrates a perspective view of an example device having paddles of adjustable widths;

FIG. 44 illustrates a perspective view of an example device having paddles of adjustable widths;

FIG. 45 illustrates a side view of an example device having a spacer of adjustable width with the spacer in a narrow configuration;

FIG. 46 illustrates a front view of the device of FIG. 45;

FIG. 47 illustrates a side view of the device of FIG. 45 with the spacer in a wide or expanded configuration;

FIG. 48 illustrates a front view of the device of FIG. 47;

FIG. 49 illustrates a front view of an example spacer frame useable as part of a device;

FIG. 50 illustrates a front view of an example device having paddles of adjustable widths with the paddles in a narrow configuration;

FIG. 51 illustrates a side view of the device of FIG. 50;

FIG. 52 illustrates a front view of the device of FIG. 50 with the paddles in a wide or expanded configuration;

FIG. 53 illustrates a side view of the device of FIG. 50;

FIG. 54 illustrates a partial perspective view of an example of a portion of a paddle structure and with adjustable width paddle frames that is in isolation of other repair device components.

FIG. 55 is a partial side view of a portion of the paddle structure and assembly shown in FIG. 54.

FIGS. 56A, 56B, 56C, and 56D illustrate partial views of implementations of paddle structures for implementing one or more guide structures thereon or therewith.

FIGS. 57A and 57B illustrate perspective views of certain components of a heart valve repair device having hinge or pivot joint portions for movement of adjustable width paddle frames from an expanded to a narrowed configuration.

FIG. 58 illustrates a perspective view of certain components of an implementation of a heart valve repair device having hinge or pivot joint portions for movement of adjustable width paddle frames from a narrowed to an expanded configuration.

FIG. 59 illustrates a perspective view of certain components of an implementation of a heart valve repair device having hinge or pivot joint portions for movement of adjustable width paddle frames from a narrowed to an expanded configuration.

FIG. 60 illustrates a perspective view of certain components of an implementation of a heart valve repair device having hinge or pivot joint portions for movement of adjustable width paddle frames from a narrowed to an expanded configuration.

FIGS. 61A and 61B illustrate perspective views of certain components of an implementation of a heart valve repair device having hinge or pivot joint portions for movement of adjustable width paddle frames from a narrowed to an expanded configuration.

FIGS. 62A-62I illustrate various views of certain components of an implementation of a heart valve repair device having hinge or pivot joint portions for movement of adjustable width paddle frames from an expanded to a narrowed configuration.

FIGS. 63A-63G illustrate various views of certain components of an implementation of a heart valve repair device having hinge or pivot joint portions for movement of adjustable width paddle frames from a narrowed to an expanded configuration.

FIGS. 64A-64B illustrate various views of certain components of an implementation of a heart valve repair device having hinge or pivot joint portions for movement of adjustable width paddle frames from a narrowed to an expanded configuration.

FIG. 65 illustrates a perspective view of certain components of an implementation of a heart valve repair device having hinge or pivot joint portions for movement of adjustable width paddle frames from a narrowed to an expanded configuration.

FIGS. 66A-66B illustrate various views of certain components of an implementation. of a heart valve repair device having hinge or pivot joint portions for movement of adjustable width paddle frames from an expanded to a narrowed configuration.

### DETAILED DESCRIPTION

The following description refers to the accompanying drawings, which illustrate example implementations of the present disclosure. Other implementations having different structures and operation do not depart from the scope of the present disclosure. The illustrations are presented at a representative scale, except where otherwise clearly indicated or apparent from the context.

Some implementations of the present disclosure are directed to systems, devices, methods, etc. for repairing a defective heart valve. For example, some implementations of devices, treatment device, repair devices, valve treatment devices, valve repair devices, implantable devices, implants, and systems (including systems for delivery thereof) are disclosed herein, and any combination of these options can be made unless specifically excluded. In other words, individual components of the disclosed devices and systems can be combined unless mutually exclusive or otherwise physically impossible. Further, the techniques, methods, operations, steps, etc. described or suggested herein or in the references incorporated herein, and any methods of using the systems, assemblies, apparatuses, devices, etc. herein, can be performed on a living subject (e.g., human, other animal, etc.) or on a simulation (e.g., a cadaver, cadaver heart, simulator, imaginary person, anthropomorphic ghost, etc.). When performed on a simulation, the body parts, e.g., heart, tissue, valve, etc., can be assumed to be simulated or can optionally be referred to as “simulated” (e.g., simulated heart, simulated tissue, simulated valve, etc.) and can optionally comprise computerized and/or physical representations of body parts, tissue, etc. The term “simulation” covers use on a cadaver, computer simulator, imaginary person (e.g., if they are just demonstrating in the air on an imaginary heart), etc.

As described herein, when one or more components are described as being connected, joined, affixed, coupled, attached, or otherwise interconnected, such interconnection can be direct as between the components or can be indirect such as through the use of one or more intermediary components. Also as described herein, reference to a “member,” “component,” or “portion” shall not be limited to a single structural member, component, or element but can include an assembly of components, members, or elements. Also as described herein, the terms “substantially” and “about” are defined as at least close to (and includes) a given value or state (preferably within 10% of, more preferably within 1% of, and most preferably within 0.1% of). The terms “clasp” and “clasp arm” are often used herein with respect to specific examples, but the terms “gripping member” and/or “gripper arm” can be used in place of and function in the same or similar ways, even if not configured in the same way as a typical clasp.

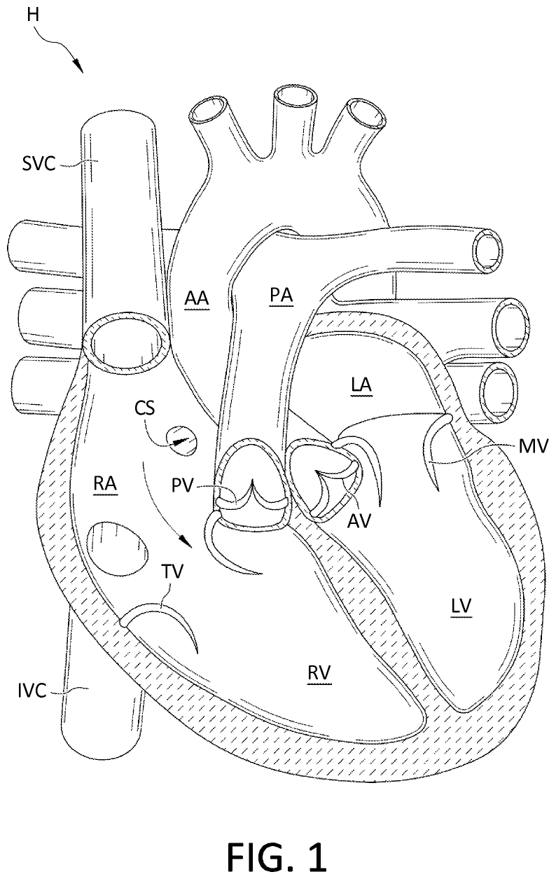

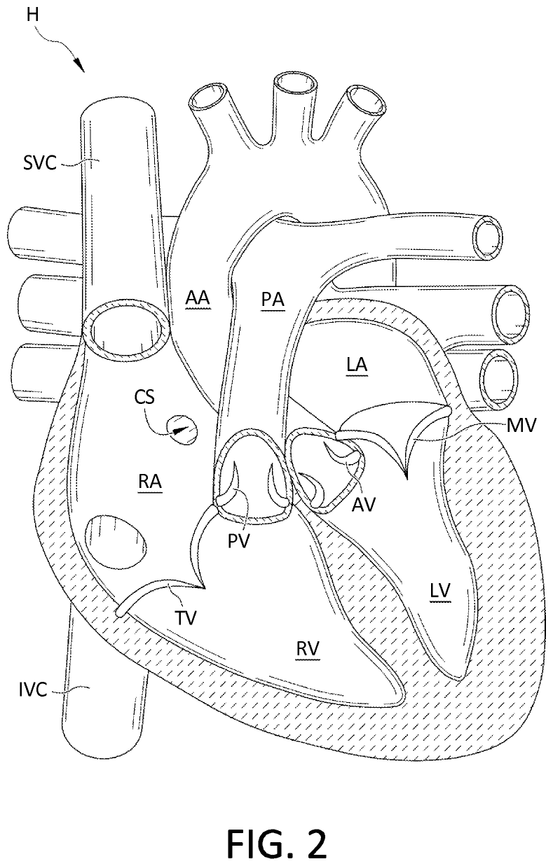

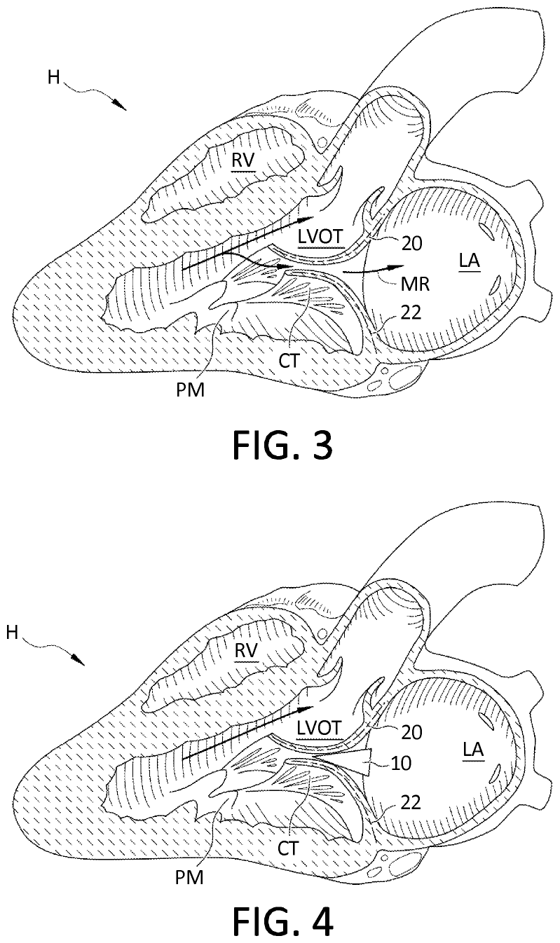

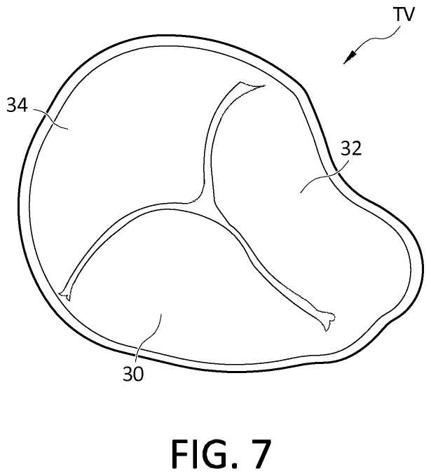

FIGS. 1 and 2 are cutaway views of the human heart H in diastolic and systolic phases, respectively. The right ventricle RV and left ventricle LV are separated from the right atrium RA and left atrium LA, respectively, by the tricuspid valve TV and mitral valve MV; i.e., the atrioventricular valves. Additionally, the aortic valve AV separates the left ventricle LV from the ascending aorta AA, and the pulmonary valve PV separates the right ventricle from the pulmonary artery PA. Each of these valves has flexible leaflets (e.g., leaflets 20, 22 shown in FIGS. 3-6 and leaflets 30, 32, 34 shown in FIG. 7) extending inward across the respective orifices that come together or “coapt” in the flow stream to form the one-way, fluid-occluding surfaces. The native valve repair and/or treatment systems of the present application are frequently described and/or illustrated with respect to the mitral valve MV. Therefore, anatomical structures of the left atrium LA and left ventricle LV will be explained in greater detail. However, the devices described herein can also be used in repairing other native valves, e.g., the devices can be used in repairing the tricuspid valve TV, the aortic valve AV, and the pulmonary valve PV.

The left atrium LA receives oxygenated blood from the lungs. During the diastolic phase, or diastole, seen in FIG. 1, the blood that was previously collected in the left atrium LA (during the systolic phase) moves through the mitral valve MV and into the left ventricle LV by expansion of the left ventricle LV. In the systolic phase, or systole, seen in FIG. 2, the left ventricle LV contracts to force the blood through the aortic valve AV and ascending aorta AA into the body. During systole, the leaflets of the mitral valve MV close to prevent the blood from regurgitating from the left ventricle LV and back into the left atrium LA and blood is collected in the left atrium from the pulmonary vein. In some implementations, the devices described by the present application are used to repair the function of a defective mitral valve MV. That is, the devices are configured to help close the leaflets of the mitral valve to prevent, inhibit or reduce blood from regurgitating from the left ventricle LV and back into the left atrium LA. Many of the devices described in the present application are designed to easily grasp and secure the native leaflets around a coaptation element or spacer that beneficially acts as a filler in the regurgitant orifice to prevent or inhibit back flow or regurgitation during systole, though this is not necessary.

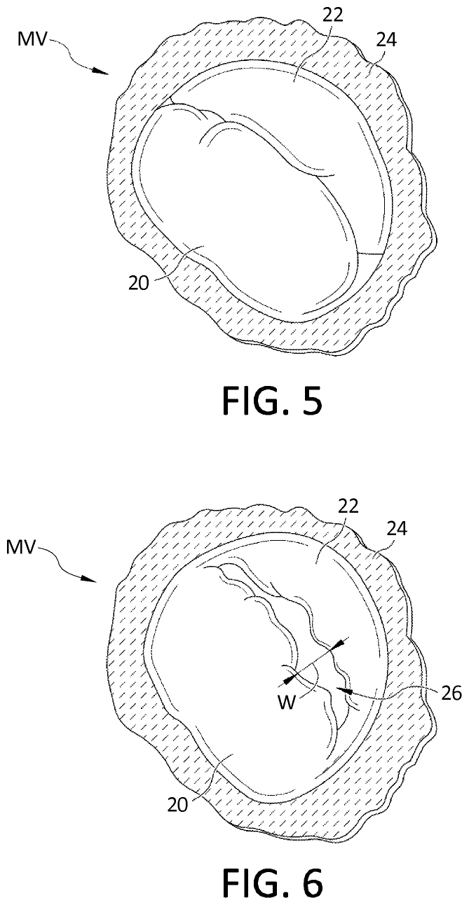

Referring now to FIGS. 1-7, the mitral valve MV includes two leaflets, the anterior leaflet 20 and the posterior leaflet 22. The mitral valve MV also includes an annulus 24 (see FIG. 5), which is a variably dense fibrous ring of tissues that encircles the leaflets 20, 22. Referring to FIGS. 3 and 4, the mitral valve MV is anchored to the wall of the left ventricle LV by chordae tendineae CT. The chordae tendineae CT are cord-like tendons that connect the papillary muscles PM (i.e., the muscles located at the base of the chordae tendineae CT and within the walls of the left ventricle LV) to the leaflets 20, 22 of the mitral valve MV. The papillary muscles PM serve to limit the movements of leaflets 20, 22 of the mitral valve MV and prevent the mitral valve MV from being reverted. The mitral valve MV opens and closes in response to pressure changes in the left atrium LA and the left ventricle LV. The papillary muscles PM do not open or close the mitral valve MV. Rather, the papillary muscles PM support or brace the leaflets 20, 22 against the high pressure needed to circulate blood throughout the body. Together the papillary muscles PM and the chordae tendineae CT are known as the subvalvular apparatus, which functions to keep the mitral valve MV from prolapsing into the left atrium LA when the mitral valve closes. As seen from a Left Ventricular Outflow Tract (LVOT) view shown in FIG. 3, the anatomy of the leaflets 20, 22 is such that the inner sides of the leaflets coapt at the free end portions and the leaflets 20, 22 start receding or spreading apart from each other. The leaflets 20, 22 spread apart in the atrial direction, until each leaflet meets with the mitral annulus.

Various disease processes can impair proper function of one or more of the native valves of the heart H. These disease processes include degenerative processes (e.g., Barlow's Disease, fibroelastic deficiency, etc.), inflammatory processes (e.g., Rheumatic Heart Disease), and infectious processes (e.g., endocarditis, etc.). In addition, damage to the left ventricle LV or the right ventricle RV from prior heart attacks (i.e., myocardial infarction secondary to coronary artery disease) or other heart diseases (e.g., cardiomyopathy, etc.) may distort a native valve's geometry, which may cause the native valve to dysfunction. However, the majority of patients undergoing valve surgery, such as surgery to the mitral valve MV, suffer from a degenerative disease that causes a malfunction in a leaflet (e.g., leaflets 20, 22) of a native valve (e.g., the mitral valve MV), which results in prolapse and regurgitation.

Generally, a native valve may malfunction in different ways: including (1) valve stenosis; and (2) valve regurgitation. Valve stenosis occurs when a native valve does not open completely and thereby causes an obstruction of blood flow. Typically, valve stenosis results from buildup of calcified material on the leaflets of a valve, which causes the leaflets to thicken and impairs the ability of the valve to fully open to permit forward blood flow. Valve regurgitation occurs when the leaflets of the valve do not close completely thereby causing blood to leak back into the prior chamber (e.g., causing blood to leak from the left ventricle to the left atrium).

There are three main mechanisms by which a native valve becomes regurgitant—or incompetent—which include Carpentier's type I, type II, and type III malfunctions. A Carpentier type I malfunction involves the dilation of the annulus such that normally functioning leaflets are distracted from each other and fail to form a tight seal (i.e., the leaflets do not coapt properly). Included in a type I mechanism malfunction are perforations of the leaflets, as are present in endocarditis. A Carpentier's type II malfunction involves prolapse of one or more leaflets of a native valve above a plane of coaptation. A Carpentier's type III malfunction involves restriction of the motion of one or more leaflets of a native valve such that the leaflets are abnormally constrained below the plane of the annulus. Leaflet restriction may be caused by rheumatic disease or dilation of a ventricle.

Referring to FIG. 5, when a healthy mitral valve MV is in a closed position, the anterior leaflet 20 and the posterior leaflet 22 coapt, which prevents blood from leaking from the left ventricle LV to the left atrium LA. Referring to FIGS. 3 and 6, mitral regurgitation MR occurs when the anterior leaflet 20 and/or the posterior leaflet 22 of the mitral valve MV is displaced into the left atrium LA during systole so that the edges of the leaflets 20, 22 are not in contact with each other. This failure to coapt causes a gap 26 between the anterior leaflet 20 and the posterior leaflet 22, which allows blood to flow back into the left atrium LA from the left ventricle LV during systole, as illustrated by the mitral regurgitation MR flow path shown in FIG. 3. Referring to FIG. 6, the gap 26 may have a width W between about 2.5 mm and about 17.5 mm, between about 5 mm and about 15 mm, between about 7.5 mm and about 12.5 mm, or about 10 mm. In some situations, the gap 26 may have a width W greater than 15 mm or even 17.5 mm. As set forth above, there are several different ways that a leaflet (e.g., leaflets 20, 22 of mitral valve MV) may malfunction which may thereby lead to valvular regurgitation.

In any of the above-mentioned situations, a device or implant is desired that is capable of engaging the anterior leaflet 20 and the posterior leaflet 22 to close the gap 26 and prevent or inhibit regurgitation of blood through the mitral valve MV. As can be seen in FIG. 4, an abstract representation of a repair or treatment device 10 (e.g., a valve treatment device, a valve repair device, an implantable device, an implant, etc.) is shown implanted between the leaflets 20, 22 such that regurgitation does not occur during systole (compare FIG. 3 with FIG. 4). In some implementations, the coaptation element (e.g., spacer, coaptation element, gap filler, membrane, sheet, plug, wedge, balloon, etc.) of the device 10 has a generally tapered or triangular shape that naturally adapts to the native valve geometry and to its expanding leaflet nature (toward the annulus). In this application, the terms spacer, coaptation element, coaptation element, gap filler, plug, etc. are used interchangeably and refer to an element that fills a portion of the space between native valve leaflets and/or that is configured such that the native valve leaflets engage or “coapt” against (e.g., such that the native leaflets coapt against the coaptation element, coaptation element, spacer, etc. instead of only against one another).

Although stenosis or regurgitation may affect any valve, stenosis is predominantly found to affect either the aortic valve AV or the pulmonary valve PV, and regurgitation is predominantly found to affect either the mitral valve MV or the tricuspid valve TV. Both valve stenosis and valve regurgitation increase the workload of the heart H and may lead to very serious conditions if left un-treated; such as endocarditis, congestive heart failure, permanent heart damage, cardiac arrest, and ultimately death. Because the left side of the heart (i.e., the left atrium LA, the left ventricle LV, the mitral valve MV, and the aortic valve AV) are primarily responsible for circulating the flow of blood throughout the body. Accordingly, because of the substantially higher pressures on the left side heart dysfunction of the mitral valve MV or the aortic valve AV is particularly problematic and often life threatening.

Malfunctioning native heart valves can either be repaired or replaced. Repair typically involves the preservation and correction of the patient's native valve. Replacement typically involves replacing the patient's native valve with a biological or mechanical substitute. Typically, the aortic valve AV and pulmonary valve PV are more prone to stenosis. Because stenotic damage sustained by the leaflets is irreversible, treatments for a stenotic aortic valve or stenotic pulmonary valve can be removal and replacement of the valve with a surgically implanted heart valve, or displacement of the valve with a transcatheter heart valve. The mitral valve MV and the tricuspid valve TV are more prone to deformation of leaflets and/or surrounding tissue, which, as described above, may prevent the mitral valve MV or tricuspid valve TV from closing properly and allows for regurgitation or back flow of blood from the ventricle into the atrium (e.g., a deformed mitral valve MV may allow for regurgitation or back flow from the left ventricle LV to the left atrium LA as shown in FIG. 3). The regurgitation or back flow of blood from the ventricle to the atrium results in valvular insufficiency. Deformations in the structure or shape of the mitral valve MV or the tricuspid valve TV are often repairable. In addition, regurgitation may occur due to the chordae tendineae CT becoming dysfunctional (e.g., the chordae tendineae CT may stretch or rupture), which allows the anterior leaflet 20 and the posterior leaflet 22 to be reverted such that blood is regurgitated into the left atrium LA. The problems occurring due to dysfunctional chordae tendineae CT can be repaired by repairing the chordae tendineae CT or the structure of the mitral valve MV (e.g., by securing the leaflets 20, 22 at the affected portion of the mitral valve).

The devices and procedures disclosed herein often make reference to repairing the structure of a mitral valve. However, it should be understood that the devices and concepts provided herein can be used to repair any native valve, as well as any component of a native valve. Such devices can be used between the leaflets 20, 22 of the mitral valve MV to prevent or inhibit regurgitation of blood from the left ventricle into the left atrium. With respect to the tricuspid valve TV (FIG. 7), any of the devices and concepts herein can be used between any two of the anterior leaflet 30, septal leaflet 32, and posterior leaflet 34 to prevent or inhibit regurgitation of blood from the right ventricle into the right atrium. In addition, any of the devices and concepts provided herein can be used on all three of the leaflets 30, 32, 34 together to prevent or inhibit regurgitation of blood from the right ventricle to the right atrium. That is, the treatment device, repair devices, implants, etc. provided herein can be centrally located between the three leaflets 30, 32, 34.

An example device (e.g., valve repair device, valve treatment device, implantable device, implant, etc.) can optionally have a coaptation element (e.g., spacer, coaptation element, gap filler, membrane, sheet, plug, wedge, balloon, etc.) and at least one anchor (e.g., one, two, three, or more). In some implementations, a device (e.g., a valve repair device, a valve treatment device, an implantable device, an implant, etc.) can have any combination or sub-combination of the features disclosed herein without a coaptation element. When included, the coaptation element (e.g., spacer, coaptation element, gap filler, membrane, sheet, plug, wedge, balloon, etc.) is configured to be positioned within the native heart valve orifice to help fill the space between the leaflets and form a more effective seal, thereby reducing or preventing or inhibiting regurgitation described above. The coaptation element can have a structure that is impervious to blood (or that resists blood flow therethrough) and that allows the native leaflets to close around the coaptation element during ventricular systole to block blood from flowing from the left or right ventricle back into the left or right atrium, respectively. The device can be configured to seal against two or three native valve leaflets; that is, the device can be used in the native mitral (bicuspid) and tricuspid valves. The coaptation element is sometimes referred to herein as a spacer because the coaptation element can fill a space between improperly functioning native leaflets (e.g., mitral leaflets 20, 22 or tricuspid leaflets 30, 32, 34) that do not close completely.

The optional coaptation element (e.g., spacer, coaptation element, gap filler, membrane, sheet, plug, wedge, balloon, etc.) can have various shapes. In some implementations, the coaptation element can have an elongated cylindrical shape having a round cross-sectional shape. In some implementations, the coaptation element can have an oval cross-sectional shape, an ovoid cross-sectional shape, a crescent cross-sectional shape, a rectangular cross-sectional shape, or various other non-cylindrical shapes. In some implementations, the coaptation element can have an atrial portion positioned in or adjacent to the atrium, a ventricular or lower portion positioned in or adjacent to the ventricle, and a side surface that extends between the native leaflets. In some implementations configured for use in the tricuspid valve, the atrial or upper portion is positioned in or adjacent to the right atrium, and the ventricular or lower portion is positioned in or adjacent to the right ventricle, and the side surfaces extend between the native tricuspid leaflets.

In some implementations, the anchor can be configured to secure the device to one or both of the native leaflets such that the coaptation element is positioned between the two native leaflets.

In some implementations, the anchor can be configured for use in the tricuspid valve, the anchor is configured to secure the device to one, two, or three of the tricuspid leaflets such that the coaptation element is positioned between the three native leaflets.

In some implementations, the anchor can attach to the coaptation element at a location adjacent the ventricular portion of the coaptation element. In some implementations, the anchor can attach to an actuation element (e.g., an actuation shaft, actuation tube, actuation wire, etc.) to which the coaptation element is also attached. In some implementations, the anchor and the coaptation element can be positioned independently with respect to each other by separately moving each of the anchor and the coaptation element along the longitudinal axis of the actuation element (e.g., actuation shaft, actuation rod, actuation tube, actuation wire, etc.). In some implementations, the anchor and the coaptation element can be positioned simultaneously by moving the anchor and the coaptation element together along the longitudinal axis of the actuation element (e.g., shaft, actuation wire, etc.). The anchor can be configured to be positioned behind a native leaflet when deployed such that the leaflet is grasped by the anchor.

The device can be configured to be deployed and/or implanted via a delivery system or other means for delivery. The delivery system can comprise one or more of a guide/delivery sheath, a delivery catheter, a steerable catheter, an implant catheter, tube, combinations of these, etc. The coaptation element and the anchor can be compressible to a radially compressed state and can be self-expandable to a radially expanded state when compressive pressure is released. The device can be configured for the anchor to be expanded radially away from the still compressed coaptation element initially in order to create a gap between the coaptation element and the anchor. A native leaflet can then be positioned in the gap. The coaptation element can be expanded radially, closing the gap between the coaptation element and the anchor and capturing the leaflet between the coaptation element and the anchor. In some implementations, the anchor and coaptation element are optionally configured to self-expand. The implantation and/or deployment methods for some implementations can be different and are more fully discussed below with respect to each implementation. Additional information regarding these and other delivery methods that can be used with the concepts herein can be found in U.S. Pat. No. 8,449,599 and U.S. Patent Application Publication Nos. 2014/0222136, 2014/0067052, 2016/0331523, PCT patent application publication Nos. WO2020/076898, WO2023/278663, WO2023/004098, WO2023/091520, WO2023/107296, WO2023/086340, WO2023/003755, and WO2022/231889 each of which is incorporated herein by reference in its entirety for all purposes. These method(s) can be performed on a living animal or on a simulation, such as on a cadaver, cadaver heart, simulator (e.g., with the body parts, heart, tissue, etc. being simulated), etc. mutatis mutandis.

The disclosed devices or implants can be configured such that the anchor is connected to a leaflet, taking advantage of the tension from native chordae tendineae to resist high systolic pressure urging the device toward the left atrium. During diastole, the devices can rely on the compressive and retention forces exerted on the leaflet that is grasped by the anchor.

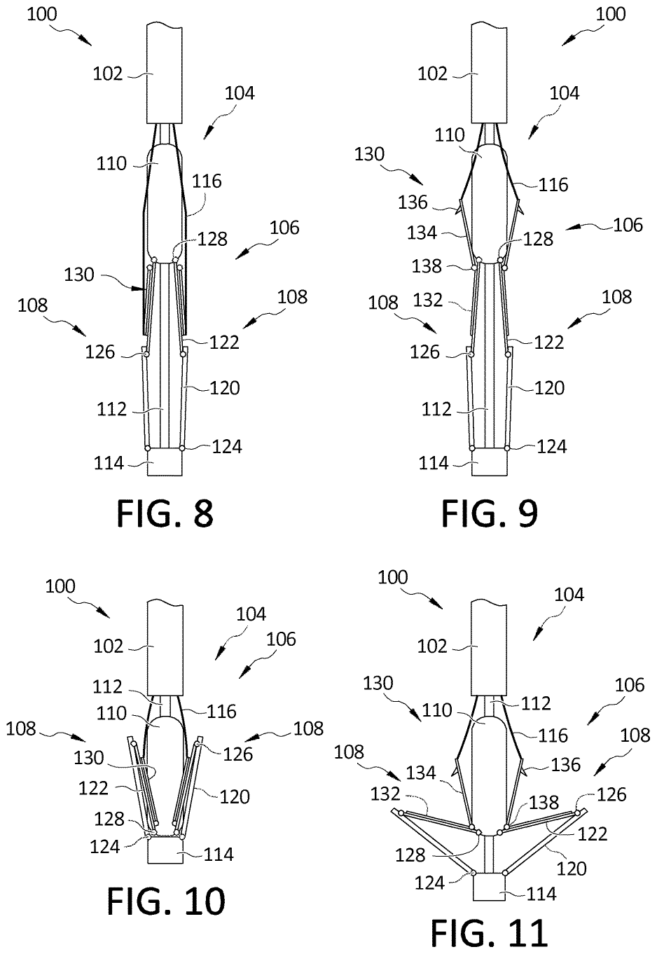

Referring now to FIGS. 8-15, a schematically illustrated device 100 (e.g., a prosthetic device, a valve repair device, valve treatment device, implantable device, implant, etc.) is shown in various stages of deployment. The device 100 and other similar devices and/or implants are described in more detail in PCT patent application publication Nos. WO2018/195215, WO2020/076898, WO2019/139904, WO2023278663, WO2023/004098, WO2023/091520, WO2023/107296, WO2023/086340, WO2023/003755, and WO2022/231889, which are incorporated herein by reference in their entirety for all purposes. The devices herein can include any other features for another device or implant discussed in the present application or the applications cited above, and the devices herein can be positioned to engage valve tissue (e.g., leaflets 20, 22, 30, 32, 34) as part of any suitable treatment and/or repair system (e.g., any treatment and/or repair system disclosed in the present application, or the applications cited herein).

The device 100 is deployed from a delivery system 102. The delivery system 102 can comprise one or more of a catheter, a sheath, a guide catheter/sheath, a delivery catheter/sheath, a steerable catheter, an implant catheter, a tube, a channel, a pathway, combinations of these, etc. The device 100 includes a coaptation portion 104 and an anchor portion 106.

In some implementations, the coaptation portion 104 of the device 100 includes a coaptation element 110 that is adapted to be deployed and/or implanted between leaflets of a native valve (e.g., a native mitral valve, native tricuspid valve, etc.) and is slidably attached to an actuation element 112 (e.g., actuation wire, shaft, tube, hypotube, line, suture, braid, etc.). The anchor portion 106 includes one or more anchors 108 that are actuatable between open and closed conditions and can take a wide variety of forms, such as, for example, paddles, gripping elements, or the like. Actuation of the actuation element 112 opens and closes the anchor portion 106 of the device 100 to grasp the native valve leaflets during deployment and/or implantation. The actuation element 112 (as well as other actuation elements disclosed herein) can take a wide variety of different forms (e.g., as a wire, rod, shaft, tube, screw, suture, line, strip, combination of these, etc.), be made of a variety of different materials, and have a variety of configurations. As one example, the actuation element can be threaded such that rotation of the actuation element moves the anchor portion 106 relative to the coaptation portion 104. Or, the actuation element can be unthreaded, such that pushing or pulling the actuation element 112 moves the anchor portion 106 relative to the coaptation portion 104.

The anchor portion 106 and/or anchors of the device 100 include outer paddles 120 and inner paddles 122 that are, in some implementations, connected between a cap 114 and a coaptation element 110 by portions 124, 126, 128. The portions 124, 126, 128 can be jointed and/or flexible to move between all of the positions described below. The interconnection of the outer paddles 120, the inner paddles 122, the coaptation element 110, and the cap 114 by the portions 124, 126, and 128 can constrain the device to the positions and movements illustrated herein.

In some implementations, the delivery system 102 includes a steerable catheter, implant catheter, and the actuation element 112 (e.g., actuation wire, shaft, tube, hypotube, line, suture, braid, etc.). These can be configured to extend through a guide catheter/sheath (e.g., a transseptal sheath, etc.). In some implementations, the actuation element 112 extends through a delivery catheter and the coaptation element 110 to the distal end (e.g., a cap 114 or other attachment portion at the distal connection of the anchor portion 106). Extending and retracting the actuation element 112 increases and decreases the spacing between the coaptation element 110 and the distal end of the device (e.g., the cap 114 or other attachment portion), respectively. In some implementations, a collar or other attachment element (e.g., clamp, clip, lock, sutures, friction fit, buckle, snap fit, lasso, etc.) removably attaches the coaptation element 110 to the delivery system 102, either directly or indirectly, so that the actuation element 112 slides through the collar or other attachment element and, in some implementations, through a coaptation element 110 during actuation to open and close the paddles 120, 122 of the anchor portion 106 and/or anchors 108.

In some implementations, the anchor portion 106 and/or anchors 108 can include attachment portions or gripping members (e.g., gripping arms, clasp arms, etc.). The illustrated gripping members can comprise clasps 130 that include a base or fixed arm 132, a moveable arm 134, optional friction-enhancing elements, other securing structures 136 (e.g., barbs, protrusions, ridges, grooves, textured surfaces, adhesive, etc.), and a joint portion 138. The fixed arms 132 are attached to the inner paddles 122. In some implementations, the fixed arms 132 are attached to the inner paddles 122 with the joint portion 138 disposed proximate the coaptation element 110. The joint portion 138 provides a spring force between the fixed and moveable arms 132, 134 of the clasp 130. The joint portion 138 can be any suitable joint, such as a flexible joint, a spring joint, a pivot joint, or the like. In some implementations, the joint portion 138 is a flexible piece of material integrally formed with the fixed and moveable arms 132, 134. The fixed arms 132 are attached to the inner paddles 122 and remain stationary or substantially stationary relative to the inner paddles 122 when the moveable arms 134 are opened to open the clasps 130 and expose the optional barbs or other friction-enhancing elements 136.

In some implementations, the clasps 130 are opened by applying tension to actuation lines 116 attached to the moveable arms 134, thereby causing the moveable arms 134 to articulate, flex, or pivot on the joint portions 138. The actuation lines 116 extend through the delivery system 102 (e.g., through a steerable catheter, an implant catheter, etc.). Other actuation mechanisms are also possible.

The actuation line 116 can take a wide variety of forms, such as, for example, a line, a suture, a wire, a rod, a catheter, or the like. The clasps 130 can be spring loaded so that in the closed position the clasps 130 continue to provide a pinching force on the grasped native leaflet. Optional barbs or other friction-enhancing elements 136 of the clasps 130 can grab, pinch, and/or pierce the native leaflets to further secure the native leaflets.

During deployment and/or implantation, the paddles 120, 122 can be opened and closed, for example, to grasp the native leaflets (e.g., native mitral valve leaflets, etc.) between the paddles 120, 122 and/or between the paddles 120, 122 and a coaptation element 110 (e.g., a spacer, plug, membrane, etc.).

The clasps 130 can be used to grasp and/or further secure the native leaflets by engaging the leaflets with optional barbs or other friction-enhancing elements 136 and pinching the leaflets between the moveable and fixed arms 134, 132. The optional barbs or other friction-enhancing elements 136 (e.g., protrusions, ridges, grooves, textured surfaces, adhesive, etc.) of the clasps 130 increase friction with the leaflets or can partially or completely puncture the leaflets.

In some implementations, the actuation lines 116 can be actuated separately (or both separately and simultaneously) so that each clasp 130 can be opened and closed separately. Separate operation allows one leaflet to be grasped at a time, or for the repositioning of a clasp 130 on a leaflet that was insufficiently grasped, without altering a successful grasp on the other leaflet. The clasps 130 can be opened and closed relative to the position of the inner paddle 122 (as long as the inner paddle is in an open or at least partially open position), thereby allowing leaflets to be grasped in a variety of positions as the particular situation requires.

Referring now to FIG. 8, the device 100 is shown in an elongated or fully open condition for deployment from a delivery catheter of the delivery system 102. The device 100 is disposed at the end of the catheter of the delivery system 102 in the fully open position. In the elongated condition the cap 114 is spaced apart from the coaptation element 110 such that the paddles 120, 122 are fully extended. In some implementations, an angle formed between the interior of the outer and inner paddles 120, 122 is approximately 180 degrees. The clasps 130 can be kept in a closed condition during deployment through the delivery system. The actuation lines 116 can extend and attach to the moveable arms 134.

Referring now to FIG. 9, the device 100 is shown in an elongated condition, similar to FIG. 8, but with the clasps 130 in a fully open position, ranging from about 140 degrees to about 200 degrees, from about 170 degrees to about 190 degrees, or about 180 degrees between fixed and moveable arms 132, 134 of the clasps 130.

Referring now to FIG. 10, the device 100 is shown in a shortened or fully closed condition. To move the device 100 from the elongated condition to the shortened condition, the actuation element 112 is retracted to pull the cap 114 towards the coaptation element 110. The connection portion(s) 126 (e.g., joint(s), flexible connection(s), etc.) between the outer paddle 120 and inner paddle 122 are constrained in movement such that compression forces acting on the outer paddle 120 from the cap 114 being retracted towards the coaptation element 110 cause the paddles or gripping elements to move radially outward. During movement from the open position to the closed position, the outer paddles 120 maintain an acute angle with the actuation element 112. The outer paddles 120 can optionally be biased toward a closed position. The inner paddles 122 during the same motion move through a considerably larger angle as they are oriented away from the coaptation element 110 in the open condition and collapse along the sides of the coaptation element 110 in the closed condition.

Referring now to FIGS. 11-13, the device 100 is shown in a partially open, grasp-ready condition. To transition from the fully closed to the partially open condition, the actuation element (e.g., actuation wire, shaft, tube, hypotube, line, suture, braid, etc.) is extended to push the cap 114 away from the coaptation element 110, thereby pulling on the outer paddles 120, which in turn pull on the inner paddles 122, causing the anchors or anchor portion 106 to partially unfold. The actuation lines 116 are also retracted to open the clasps 130 so that the leaflets can be grasped. In some implementations, the one or more inner and outer paddles, e.g., the pair of inner and outer paddles 122, 120, are moved in unison, rather than independently, by a single actuation element 112. Also, the positions of the clasps 130 are dependent on the positions of the paddles 122, 120. For example, referring to FIG. 10 closing the paddles 122, 120 also closes the clasps. In some implementations, the paddles 120, 122 can be independently controllable. In the example illustrated by FIG. 15, the device 100 can have two actuation elements 111, 113 and two independent caps 115, 117 (or other attachment portions), such that one independent actuation element (e.g., actuation wire, shaft, tube, hypotube, line, suture, braid, etc.) and cap (or other attachment portion) are used to control one paddle, and the other independent actuation element and cap (or other attachment portion) are used to control the other paddle.

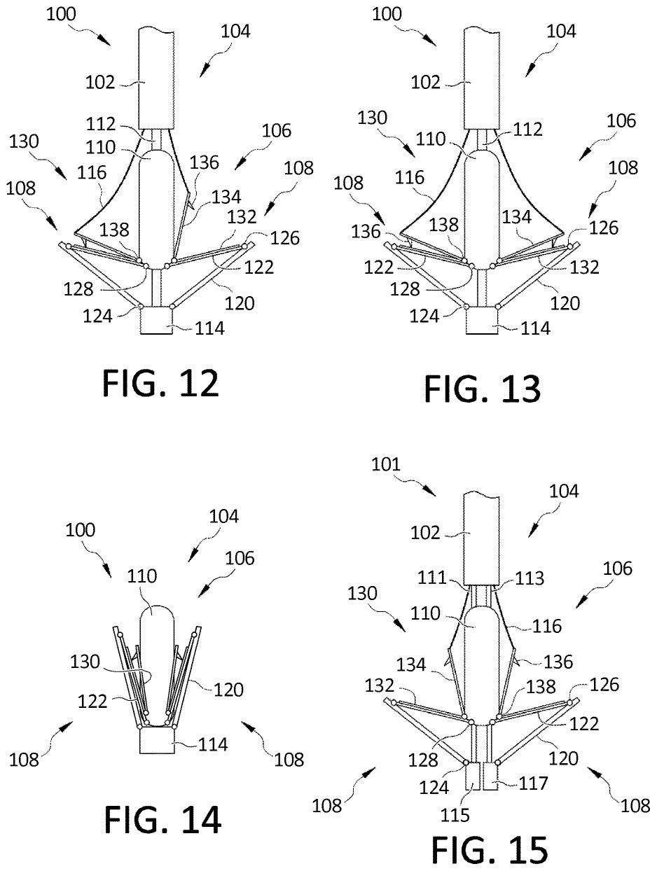

Referring now to FIG. 12, one of the actuation lines 116 is extended to allow one of the clasps 130 to close. Referring now to FIG. 13, the other actuation line 116 is extended to allow the other clasp 130 to close. Either or both of the actuation lines 116 can be repeatedly actuated to repeatedly open and close the clasps 130.

Referring now to FIG. 14, the device 100 is shown in a fully closed and deployed condition. The delivery system 102 and actuation element 112 are retracted and the paddles 120, 122 and clasps 130 remain in a fully closed position. Once deployed, the device 100 can be maintained in the fully closed position with a mechanical latch or can be biased to remain closed through the use of spring materials, such as steel, other metals, plastics, composites, etc. or shape-memory alloys such as Nitinol. For example, the connection portions 124, 126, 128, the joint portions 138, and/or the inner and outer paddles 122, and/or an additional biasing component (not shown) can be formed of metals such as steel or shape-memory alloy, such as Nitinol—produced in a wire, sheet, tubing, or laser sintered powder—and are biased to hold the outer paddles 120 closed around the coaptation element 110 and the clasps 130 pinched around native leaflets. Similarly, the fixed and moveable arms 132, 134 of the clasps 130 are biased to pinch the leaflets. In some implementations, the attachment or connection portions 124, 126, 128, joint portions 138, and/or the inner and outer paddles 122, and/or an additional biasing component (not shown) can be formed of any other suitably elastic material, such as a metal or polymer material, to maintain the device 100 in the closed condition after deployment and/or implantation.

FIG. 15 illustrates an example where the paddles 120, 122 are independently controllable. The device 101 illustrated by FIG. 15 can be the same as or similar to the device illustrated by FIG. 11, except the device 100 of FIG. 15 includes an actuation element that is configured as two independent actuation elements 111, 113 that are coupled to two independent caps 115, 117. To transition a first inner paddle 122 and a first outer paddle 120 from the fully closed to the partially open condition, the actuation element 111 is extended to push the cap 115 away from the coaptation element 110, thereby pulling on the outer paddle 120, which in turn pulls on the inner paddle 122, causing the first anchor 108 to partially unfold. To transition a second inner paddle 122 and a second outer paddle 120 from the fully closed to the partially open condition, the actuation element 113 is extended to push the cap 115 away from the spacer or coaptation element 110, thereby pulling on the outer paddle 120, which in turn pulls on the inner paddle 122, causing the second anchor 108 to partially unfold. The independent paddle control illustrated by FIG. 15 can be implemented on any of the devices disclosed by the present application. For comparison, in the example illustrated by FIG. 11, the pair of inner and outer paddles 122, 120 are moved in unison, rather than independently, by a single actuation element 112.

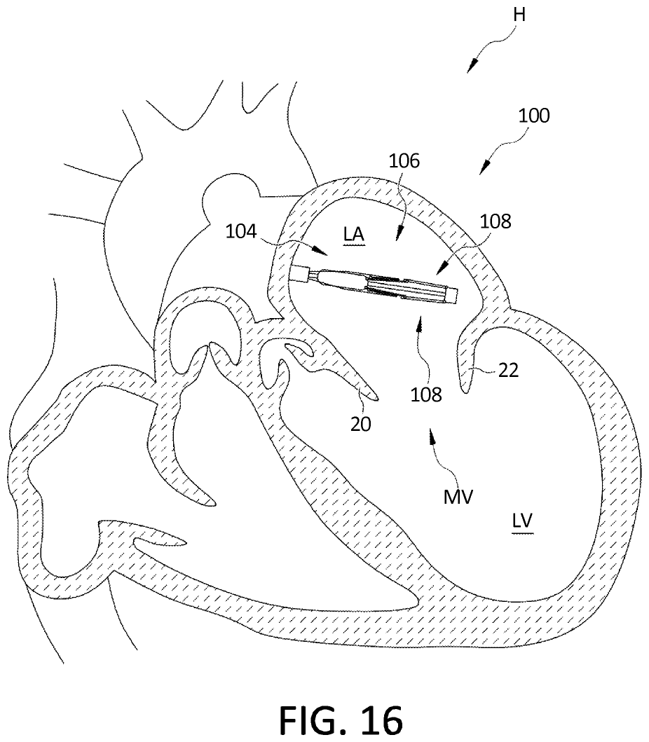

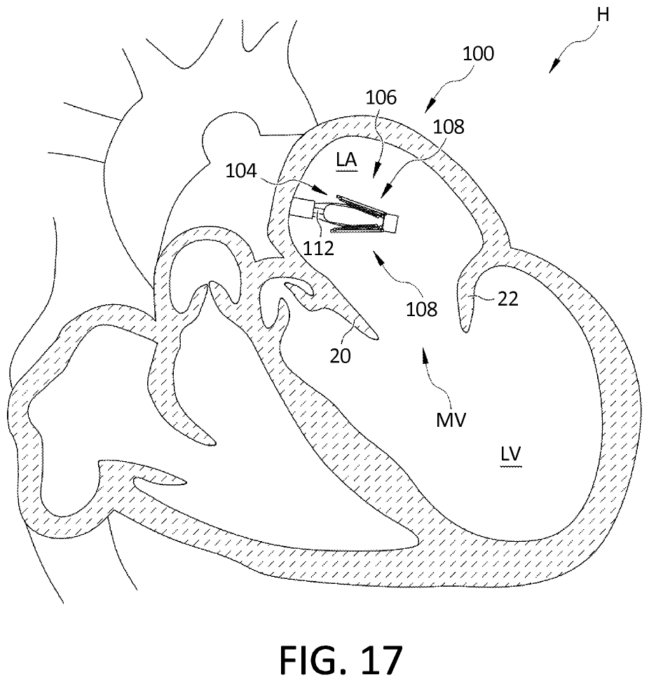

Referring now to FIGS. 16-21, the device 100 of FIGS. 8-14 is shown being delivered and deployed within the native mitral valve MV of the heart H. Referring to FIG. 16, a delivery sheath/catheter is inserted into the left atrium LA through the septum and the implant/device 100 is deployed from the delivery catheter/sheath in the fully open condition as illustrated in FIG. 16. The actuation element 112 is then retracted to move the implant/device into the fully closed condition shown in FIG. 17.

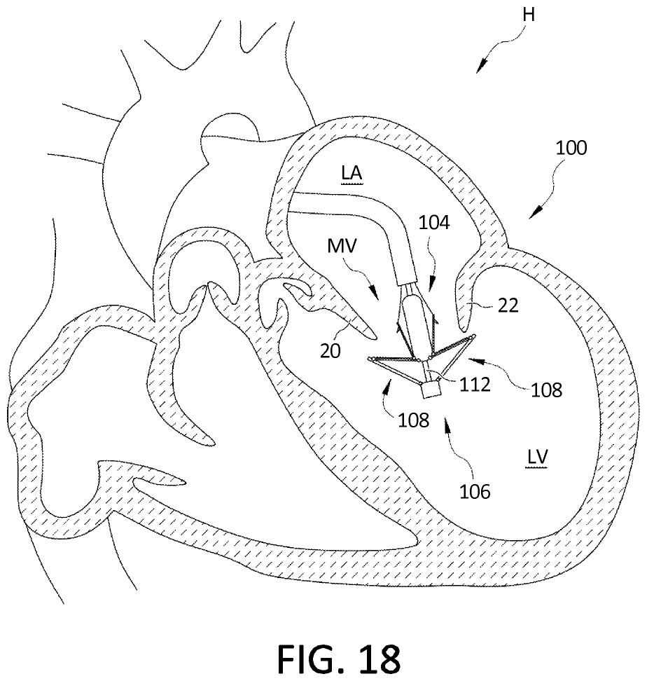

As can be seen in FIG. 18, the implant/device is moved into position within the mitral valve MV into the ventricle LV and partially opened so that the leaflets 20, 22 can be grasped. For example, a steerable catheter can be advanced and steered or flexed to position the steerable catheter as illustrated by FIG. 18. The device or implant catheter connected to the implant/device can be advanced from inside the steerable catheter to position the implant as illustrated by FIG. 18.

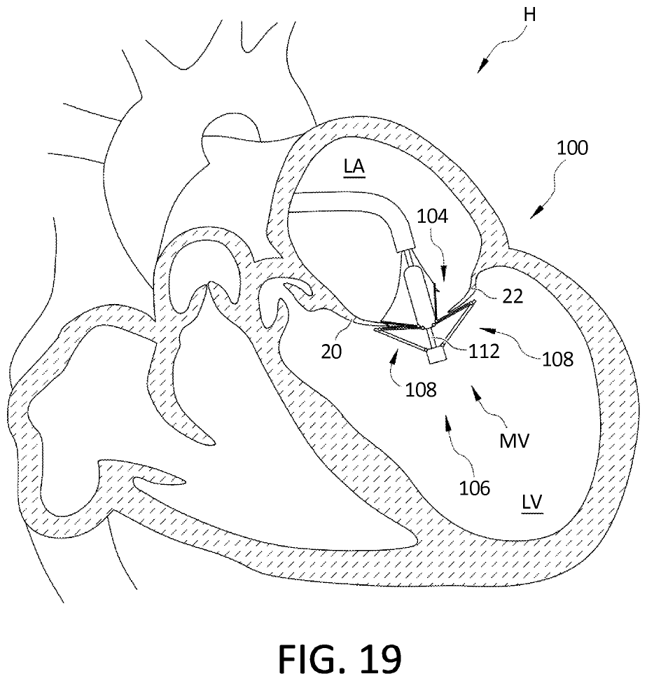

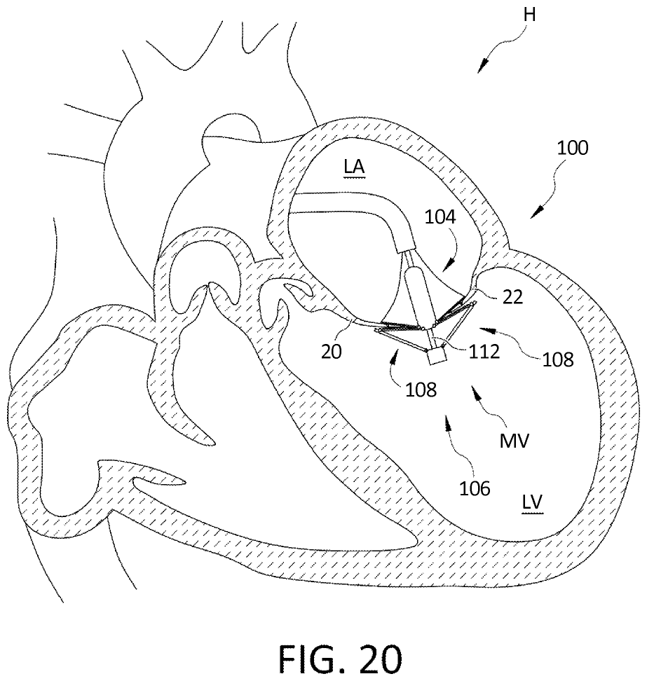

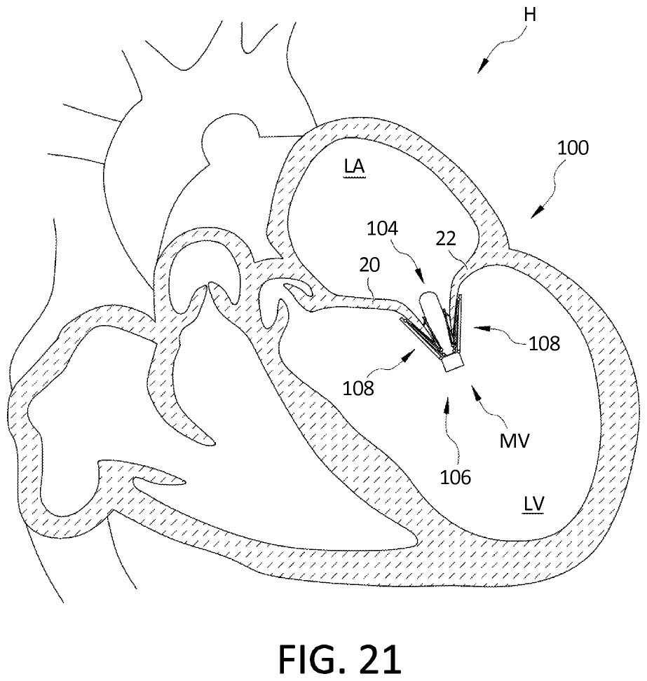

Referring now to FIG. 19, the device catheter can be retracted into the steerable catheter to position the mitral valve leaflets 20, 22 in the clasps 130. An actuation line 116 is extended to close one of the clasps 130, capturing a leaflet 20. FIG. 20 shows the other actuation line 116 being then extended to close the other clasp 130, capturing the remaining leaflet 22. Lastly, as can be seen in FIG. 21, the delivery system 102 (e.g., steerable catheter, implant catheter, etc.), actuation element 112 and actuation lines 116 are then retracted and the device 100 is fully closed and deployed in the native mitral valve MV.

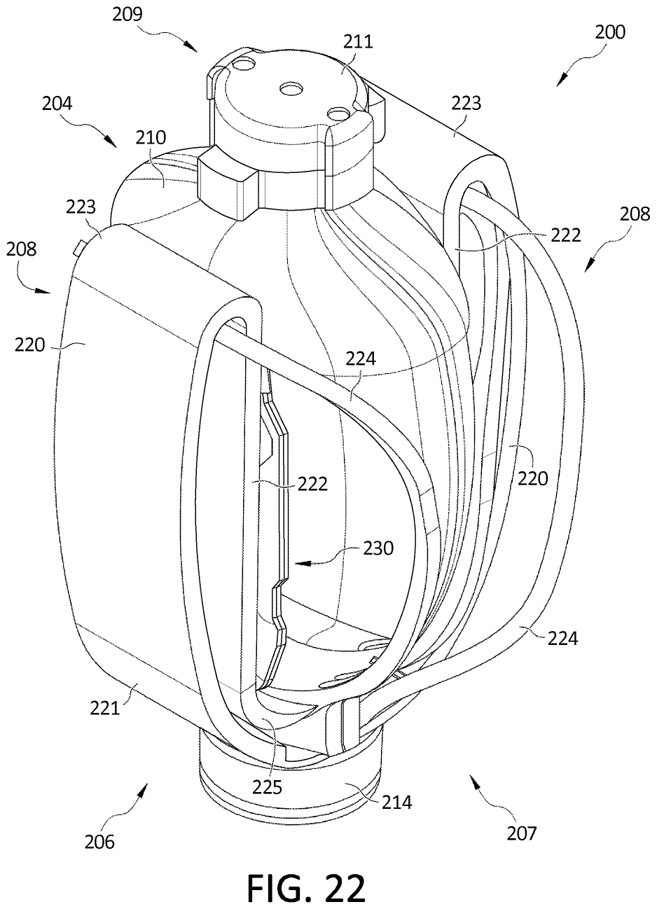

Any of the features disclosed by the present application can be used in a wide variety of different treatment devices and/or repair devices. FIGS. 22-24 illustrate examples of valve treatment and/or repair devices that can be modified to include any of the features disclosed by the present application. Any combination or sub-combination of the features disclosed by the present application can be combined with, substituted for, and/or added to any combination or sub-combination of the features of the devices illustrated by FIGS. 8-24.

Referring now to FIG. 22, an example of a device 200 (e.g., treatment device, repair device, implantable device, implant, etc.) is shown. The device 200 can be configured as an implantable device or implant or other valve treatment device (e.g., one that does not necessarily remain implanted). The device 200 is one of the many different configurations that the device 100 that is schematically illustrated in FIGS. 8-14 can take. The device 200 can include any other features for a device or implant discussed in the present application, and the device 200 can be positioned to engage valve tissue 20, 22 as part of any suitable treatment and/or repair system (e.g., any treatment and/or repair system disclosed in the present application, or the applications cited herein). The device/implant 200 can be a prosthetic spacer device, valve repair device, treatment device, or another type of implant that attaches to leaflets of a native valve.

In some implementations, the device 200 includes a coaptation portion 204, a proximal or attachment portion 209, an anchor portion 206, and a distal portion 207. In some implementations, the coaptation portion 204 of the device optionally includes a coaptation element 210 (e.g., a spacer, coaptation element, plug, membrane, sheet, gap filler, plug, wedge, balloon, etc.) for deployment and/or implantation between leaflets of a native valve. In some implementations, the anchor portion 206 includes a plurality of anchors 208. The anchors can be configured in a variety of ways. In some implementations, each anchor 208 includes outer paddles 220, inner paddles 222, paddle extension members or paddle frames 224, and clasps 230. In some implementations, the attachment portion 209 includes a first or proximal collar 211 (or other attachment element) for engaging with a capture mechanism of a delivery system. A delivery system for the device 200 can be the same as or similar to delivery system 102 described above and can comprise one or more of a catheter, a sheath, a guide catheter/sheath, a delivery catheter/sheath, a steerable catheter, an implant catheter, a tube, a channel, a pathway, combinations of these, etc. The capture mechanism can be configured in a variety of ways and, in some implementations, can comprise one or more of a clamp, clip, pin, suture, line, lasso, noose, snare, buckle, lock, latch, etc.

In some implementations, the coaptation element 210 and paddles 220, 222 are formed from a flexible material that can be a metal fabric, such as a mesh, woven, braided, or formed in any other suitable way or a laser cut or otherwise cut flexible material. The material can be cloth, shape-memory alloy wire—such as Nitinol—to provide shape-setting capability, or any other flexible material suitable for deployment and/or implantation in the human body.

An actuation element (e.g., actuation wire, shaft, tube, hypotube, line, suture, braid, etc.) can extend from a delivery system (not shown) to engage and enable actuation of the device or implant 200. In some implementations, the actuation element extends through the proximal collar 211, and spacer or coaptation element 210 to engage a cap 214 of the distal portion 207. The actuation element can be configured to removably engage the cap 214 with a threaded connection, or the like, so that the actuation element can be disengaged and removed from the device 200 after implantation.

The coaptation element 210 extends from the proximal collar 211 (or other attachment element) to the inner paddles 222. In some implementations, the coaptation element 210 has a generally elongated and round shape, though other shapes and configurations are possible. In some implementations, the coaptation element 210 has an elliptical shape or cross-section when viewed from above and has a tapered shape or cross-section when seen from a front view and a round shape or cross-section when seen from a side view. A blend of these three geometries can result in the three-dimensional shape of the illustrated coaptation element 210 that achieves the benefits described herein. The round shape of the coaptation element 210 can also be seen, when viewed from above, to substantially follow or be close to the shape of the paddle frames 224.

The size and/or shape of the coaptation element 210 can be selected to minimize the number of implants that a single patient will require (preferably one), while at the same time maintaining low transvalvular gradients. In some implementations, the anterior-posterior distance at the top of the coaptation element is about 5 mm, and the medial-lateral distance of the coaptation element at its widest is about 10 mm. In some implementations, the overall geometry of the device 200 can be based on these two dimensions and the overall shape strategy described above. It should be readily apparent that the use of other anterior-posterior distance anterior-posterior distance and medial-lateral distance as starting points for the device will result in a device having different dimensions. Further, using other dimensions and the shape strategy described above will also result in a device having different dimensions.

In some implementations, the outer paddles 220 are jointably attached to the cap 214 of the distal portion 207 by connection portions 221 and to the inner paddles 222 by connection portions 223. The inner paddles 222 are jointably attached to the coaptation element by connection portions 225. In this manner, the anchors 208 are configured similar to legs in that the inner paddles 222 are like upper portions of the legs, the outer paddles 220 are like lower portions of the legs, and the connection portions 223 are like knee portions of the legs.

In some implementations, the inner paddles 222 are stiff, relatively stiff, rigid, have rigid portions and/or are stiffened by a stiffening member or a fixed portion of the clasps 230. The inner paddle 222, the outer paddle 220, and the coaptation element can all be interconnected as described herein.

In some implementations, the paddle frames 224 are attached to the cap 214 at the distal portion 207 and extend to the connection portions 223 between the inner and outer paddles 222, 220. In some implementations, the paddle frames 224 are formed of a material that is more rigid and stiff than the material forming the paddles 222, 220 so that the paddle frames 224 provide support for the paddles 222, 220.

The paddle frames 224 can provide additional pinching force between the inner paddles 222 and the coaptation element 210 and assist in wrapping the leaflets around the sides of the coaptation element 210. That is, the paddle frames 224 can be configured with a round three-dimensional shape extending from the cap 214 to the connection portions 223 of the anchors 208. The connections between the paddle frames 224, the outer and inner paddles 220, 222, the cap 214, and the coaptation element 210 can constrain each of these parts to the movements and positions described herein. In particular the connection portion 223 is constrained by its connection between the outer and inner paddles 220, 222 and by its connection to the paddle frame 224. Similarly, the paddle frame 224 is constrained by its attachment to the connection portion 223 (and thus the inner and outer paddles 222, 220) and to the cap 214.

The wide configuration of the paddle frames 224 provides increased surface area compared to the inner paddles 222 alone. The increased surface area can distribute the clamping force of the paddles 220 and paddle frames 224 against the native leaflets over a relatively larger surface of the native leaflets in order to further protect the native leaflet tissue.

Additional features of the device 200, modified versions of the device, delivery systems for the device, and methods for using the device and delivery system are disclosed by Patent Cooperation Treaty International Application No. PCT/US2018/028189 (International Publication No. WO 2018/195215) and the other applications incorporated herein. Any combination or sub-combination of the features disclosed by the present application can be combined with any combination or sub-combination of the features disclosed by Patent Cooperation Treaty International Application No. PCT/US2018/028189 (International Publication No. WO 2018/195215) and/or the other applications incorporated herein. Patent Cooperation Treaty International Application No. PCT/US2018/028189 (International Publication No. WO 2018/195215) is incorporated herein by reference in its entirety.

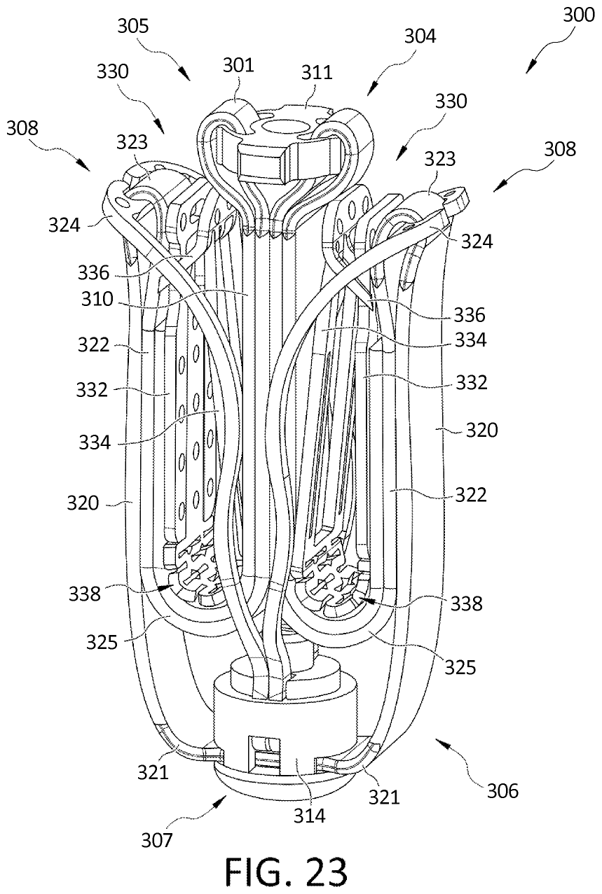

Referring now to FIG. 23, an example of a device 300 (e.g., a valve repair device, a valve treatment device, an implantable device, an implant, etc.) is shown. The device 300 is one of the many different configurations that the device 100 that is schematically illustrated in FIGS. 8-14 can take. The device 300 can include any other features for a device or implant discussed in the present application, and the device 300 can be positioned to engage valve tissue 20, 22 as part of any suitable treatment and/or repair system (e.g., any treatment and/or repair system disclosed in the present application, or the applications cited herein).

The device or implant 300 includes a proximal or attachment portion 305, an anchor portion 306, and a distal portion 307. In some implementations, the device/implant 300 includes a coaptation portion 304, and the coaptation portion 304 can optionally include a coaptation element 310 (e.g., spacer, plug, membrane, sheet, etc.) for deployment and/or implantation between the leaflets 20, 22 of the native valve. In some implementations, the anchor portion 306 includes a plurality of anchors 308. In some implementations, each anchor 308 can include one or more paddles, e.g., outer paddles 320, inner paddles 322, paddle extension members or paddle frames 324. The anchors can also include and/or be coupled to clasps 330. In some implementations, the attachment portion 305 includes a first or proximal collar 311 (or other attachment element) for engaging with a capture mechanism of a delivery system.

The anchors 308 can be attached to the other portions of the device and/or to each other in a variety of different ways (e.g., directly, indirectly, welding, sutures, adhesive, links, latches, integrally formed, a combination of some or all of these, etc.). In some implementations, the anchors 308 are attached to a coaptation element 310 by connection portions 325 and to a cap 314 by connection portions 321.

The anchors 308 can comprise first portions or outer paddles 320 and second portions or inner paddles 322 separated by connection portions 323. The connection portions 323 can be attached to paddle frames 324 that are hingeably attached to a cap 314 or other attachment portion. In this manner, the anchors 308 are configured similar to legs in that the inner paddles 322 are like upper portions of the legs, the outer paddles 320 are like lower portions of the legs, and the connection portions 323 are like knee portions of the legs.

In implementations with a coaptation element 310, the coaptation element 310 and the anchors 308 can be coupled together in various ways. As shown in the illustrated example, the coaptation element 310 and the anchors 308 can be coupled together by integrally forming the coaptation element 310 and the anchors 308 as a single, unitary component. This can be accomplished, for example, by forming the coaptation element 310 and the anchors 308 from a continuous strip 301 of a braided or woven material, such as braided or woven nitinol wire. In the illustrated example, the coaptation element 310, the outer paddle portions 320, the inner paddle portions 322, and the connection portions 321, 323, 325 are formed from a continuous strip 301.

Like the anchors 208 of the device 200 described above, the anchors 308 can be configured to move between various configurations by axially moving the distal end of the device (e.g., cap 314, etc.) relative to the proximal end of the device (e.g., proximal collar 311 or other attachment element, etc.). This movement can be along a longitudinal axis extending between the distal end (e.g., cap 314, etc.) and the proximal end (e.g., collar 311 or other attachment element, etc.) of the device.

In some implementations, in the straight configuration, the paddle portions 320, 322 are aligned or straight in the direction of the longitudinal axis of the device. In some implementations, the connection portions 323 of the anchors 308 are adjacent the longitudinal axis of the spacer or coaptation element 310. From the straight configuration, the anchors 308 can be moved to a fully folded configuration (e.g., FIG. 23), e.g., by moving the proximal end and distal end toward each other and/or toward a midpoint or center of the device.

In some implementations, the clasps comprise a moveable arm coupled to an anchor. In some implementations, the clasps 330 include a base or fixed arm 332, a moveable arm 334, optional barbs/friction-enhancing elements 336, and a joint portion 338. The fixed arms 332 are attached to the inner paddles 322, with the joint portion 338 disposed proximate the coaptation element 310. The joint portion 338 is spring-loaded so that the fixed and moveable arms 332, 334 are biased toward each other when the clasp 330 is in a closed condition.

The fixed arms 332 are attached to the inner paddles 322 through holes or slots with sutures. The fixed arms 332 can be attached to the inner paddles 322 with any suitable means, such as screws or other fasteners, crimped sleeves, mechanical latches or snaps, welding, adhesive, or the like. The fixed arms 332 remain substantially stationary relative to the inner paddles 322 when the moveable arms 334 are opened to open the clasps 330 and expose the optional barbs 336. The clasps 330 are opened by applying tension to actuation lines attached to the moveable arms 334, thereby causing the moveable arms 334 to articulate, pivot, and/or flex on the joint portions 338.

In short, the device 300 can be the same as or similar in configuration and operation to the device 200 described above, except that the coaptation element 310, outer paddles 320, inner paddles 322, and connection portions 321, 323, 325 are formed from the single strip of material 301. In some implementations, the strip of material 301 is attached to the proximal collar 311, cap 314, and paddle frames 324 by being woven or inserted through openings in the proximal collar 311, cap 314, and paddle frames 324 that are configured to receive the continuous strip of material 301. The continuous strip 301 can be a single layer of material or can include two or more layers. In some implementations, portions of the device 300 have a single layer of the strip of material 301 and other portions are formed from multiple overlapping or overlying layers of the strip of material 301.

For example, FIG. 23 shows a coaptation element 310 and inner paddles 322 formed from multiple overlapping layers of the strip of material 301. The single continuous strip of material 301 can start and end in various locations of the device 300. The ends of the strip of material 301 can be in the same location or different locations of the device 300. For example, in the illustrated example of FIG. 23, the strip of material 301 begins and ends in the location of the inner paddles 322.

As with the device 200 described above, the size of the coaptation element 310 can be selected to minimize the number of implants that a single patient will require (preferably one), while at the same time maintaining low transvalvular gradients. In particular, forming many components of the device 300 from the strip of material 301 allows the device 300 to be made smaller than the device 200. For example, in some implementations, the anterior-posterior distance at the top of the coaptation element 310 is less than 2 mm, and the medial-lateral distance of the device 300 (i.e., the width of the paddle frames 324 which are wider than the coaptation element 310) at its widest is about 5 mm.

Additional features of the device 300, modified versions of the device, delivery systems for the device, and methods for using the device and delivery system are disclosed by Patent Cooperation Treaty International Application No. PCT/US2019/055320 (International Publication No. WO 2020/076898) and/or any other applications incorporated herein. Any combination or sub-combination of the features disclosed by the present application can be combined with any combination or sub-combination of the features disclosed by Patent Cooperation Treaty International Application No. PCT/US2019/055320 (International Publication No. WO 2020/076898) and/or any other applications incorporated herein. Patent Cooperation Treaty International Application No. PCT/US2019/055320 (International Publication No. WO 2020/076898) is incorporated herein by reference in its entirety.

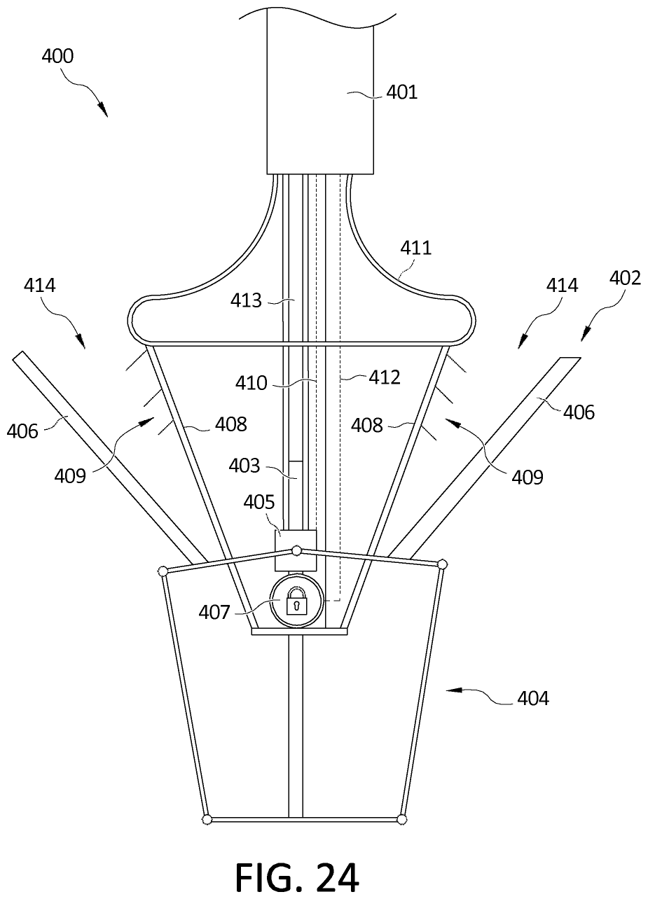

FIG. 24 illustrates an example of one of the many treatment and/or repair systems 400 for treating and/or repairing a native valve of a patient that the concepts of the present application can be applied to. The treatment and/or repair system 400 includes a delivery device 401 and a treatment and/or repair device 402.

In some implementations, the treatment device or repair device 402 includes a base assembly 404, one or more paddles 406 (e.g., a paddle, at least one paddle, a pair of paddles, three paddles, etc.), and one or more gripping members 408 (e.g., a gripping member, at least one gripping member, a pair of gripping members, three gripping members, etc.). The gripping members can be configured in a variety of ways and can comprise, for example, clasps, clasp arms, grippers, gripping arms, latches, etc.

In some implementations, the paddles 406 can be integrally formed with the base assembly. For example, the paddles 406 can be formed as extensions of links of the base assembly. In the illustrated example, the base assembly 404 of the device 402 has a shaft 403, a coupler 405 configured to move along the shaft, and a lock 407 configured to lock the coupler in a stationary position on the shaft. The coupler 405 is mechanically connected to the paddles 406, such that movement of the coupler 405 along the shaft 403 causes the paddles to move between an open position and a closed position. In this way, the coupler 405 serves as a means for mechanically coupling the paddles 406 to the shaft 403 and, when moving along the shaft 403, for causing the paddles 406 to move between their open and closed positions.

In some implementations, the gripping members 408 are pivotally connected to the base assembly 404 (e.g., the gripping members 408 can be pivotally connected to the shaft 403, or any other suitable member of the base assembly), such that the gripping members can be moved to adjust the width of the opening 414 between the paddles 406 and the gripping members 408. The gripping member 408 can include an optional barbed portion 409 for attaching the gripping members to valve tissue when the device 402 is attached to the valve tissue. When the paddles 406 are in the closed position, the paddles engage the gripping members 408, such that, when valve tissue is attached to the barbed portion 409 of the gripping members, the paddles secure the device 402 to the valve tissue. In some implementations, the gripping members 408 are configured to engage the paddles 406 such that the barbed portion 409 engages the valve tissue member and the paddles 406 to secure the device 402 to the valve tissue member. For example, in certain situations, it can be advantageous to have the paddles 406 maintain an open position and have the gripping members 408 move outward toward the paddles 406 to engage valve tissue and the paddles 406.

While the example shown in FIG. 24 illustrates a pair of paddles 406 and a pair of gripping members 408, it should be understood that the device 402 can include any suitable number of paddles and gripping members.

In some implementations, the system 400 includes a placement shaft 413 that is removably attached to the shaft 403 of the base assembly 404 of the device 402. In some implementations, after the device 402 is secured to valve tissue, the placement shaft 413 can be removed from the shaft 403 to remove the device 402 from the remainder of the treatment and/or repair system 400, such that the device 402 can remain attached to the valve tissue, and the delivery device 401 can be removed from a patient's body.

The treatment and/or repair system 400 can also include a paddle control mechanism 410, a gripper control mechanism 411, and a lock control mechanism 412. The paddle control mechanism 410 is mechanically attached to the coupler 405 to move the coupler along the shaft, which causes the paddles 406 to move between the open and closed positions. The paddle control mechanism 410 can take any suitable form, and can comprise, for example, a shaft, wire, tube, hypotube, rod, suture, line, etc. For example, the paddle control mechanism can comprise a hollow shaft, a catheter tube or a sleeve that fits over the placement shaft 413 and the shaft 403 and is connected to the coupler 405.

The gripper control mechanism 411 is configured to move the gripping members 408 such that the width of the opening 414 between the gripping members and the paddles 406 can be altered. The gripper control mechanism 411 can take any suitable form, such as, for example, a line, a suture or wire, a rod, a catheter, a tube, a hypotube, etc.

The lock control mechanism 412 is configured to lock and unlock the lock. The lock 407 locks the coupler 405 in a stationary position with respect to the shaft 403 and can take a wide variety of different forms and the type of lock control mechanism 412 can be dictated by the type of lock used. In examples in which the lock 407 includes a pivotable plate, the lock control mechanism 412 is configured to engage the pivotable plate to move the plate between the tilted and substantially non-tilted positions. The lock control mechanism 412 can be, for example, a rod, a suture, a wire, or any other member that is capable of moving a pivotable plate of the lock 407 between a tilted and substantially non-tilted position.

The device 402 is movable from an open position to a closed position. The base assembly 404 includes links that are moved by the coupler 405. The coupler 405 is movably attached to the shaft 403. In order to move the device from the open position to the closed position, the coupler 405 is moved along the shaft 403, which moves the links.

The gripper control mechanism 411 is moves the gripping members 408 to provide a wider or a narrower gap at the opening 414 between the gripping members and the paddles 406. In the illustrated example, the gripper control mechanism 411 includes a line, such as a suture, a wire, etc. that is connected to an opening in an end of the gripping members 408. When the line(s) is pulled, the gripping members 408 move inward, which causes the opening 414 between the gripping members and the paddles 406 to become wider.

In order to move the device 402 from the open position to the closed position, the lock 407 is moved to an unlocked condition by the lock control mechanism 412. Once the lock 407 is in the unlocked condition, the coupler 405 can be moved along the shaft 403 by the paddle control mechanism 410.

After the paddles 406 are moved to the closed position, the lock 407 is moved to the locked condition by the lock control mechanism 412 to maintain the device 402 in the closed position. After the device 402 is maintained in the locked condition by the lock 407, the device 402 is removed from the delivery device 401 by disconnecting the shaft 403 from the placement shaft 413. In addition, the device 402 is disengaged from the paddle control mechanism 410, the gripper control mechanism 411, and the lock control mechanism 412.

Additional features of the device 402, modified versions of the device, delivery systems for the device, and methods for using the device and delivery system are disclosed by Patent Cooperation Treaty International Application No. PCT/US2019/012707 (International Publication No. WO 2019139904) and/or any other applications incorporated herein. Any combination or sub-combination of the features disclosed by the present application can be combined with any combination or sub-combination of the features disclosed by Patent Cooperation Treaty International Application No. PCT/US2019/012707 (International Publication No. WO 2019139904) and/or any other applications incorporated herein. Patent Cooperation Treaty International Application No. PCT/US2019/012707 (International Publication No. WO 2019139904) is incorporated herein by reference in its entirety.

Clasps or leaflet gripping devices disclosed herein can take a wide variety of different forms. Examples of clasps are disclosed by Patent Cooperation Treaty International Application No. PCT/US2018/028171 (International Publication No. WO 2018195201). Any combination or sub-combination of the features disclosed by the present application can be combined with any combination or sub-combination of the features disclosed by Patent Cooperation Treaty International Application No. PCT/US2018/028171 (International Publication No. WO 2018195201). Patent Cooperation Treaty International Application No. PCT/US2018/028171 (International Publication No. WO 2018195201) is incorporated herein by reference in its entirety.

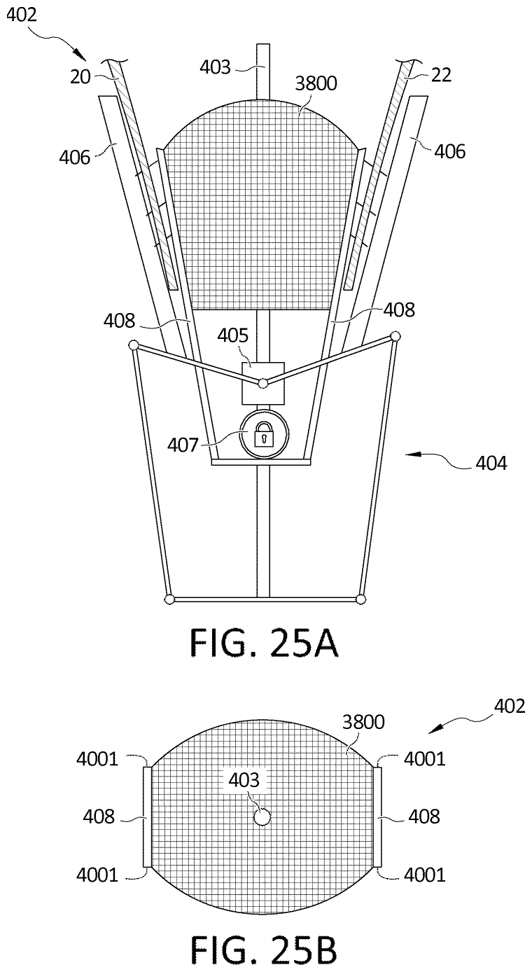

Referring to FIGS. 25A-25B, an example implementation of a treatment and/or repair device 402 has a coaptation element 3800. The device 402 can have the same configuration as the device illustrated by FIG. 24 with the addition of the coaptation element. The coaptation element 3800 can take a wide variety of different forms. The coaptation element 3800 can be compressible and/or expandable. For example, the coaptation element can be compressed to fit inside one or more catheters of a delivery system, can expand when moved out of the one or more catheters, and/or can be compressed by the paddles 406 to adjust the size of the coaptation element. In the example illustrated by FIGS. 25A and 25B, the size of the coaptation element 3800 can be reduced by squeezing the coaptation element with the paddles 406 and can be increased by moving the paddles 406 away from one another. The coaptation element 3800 can extend past outer edges 4001 of the gripping members or clasps 408 as illustrated for providing additional surface area for closing the gap of a mitral valve.

The coaptation element 3800 can be coupled to the device 402 in a variety of different ways. For example, the coaptation element 3800 can be fixed to the shaft 403, can be slidably disposed around the shaft, can be connected to the coupler 405, can be connected to the lock 407, and/or can be connected to a central portion of the clasps or gripping members 408. In some implementations, the coupler 405 can take the form of the coaptation element 3800. That is, a single element can be used as the coupler 405 that causes the paddles 406 to move between the open and closed positions and the coaptation element 3800 that closes the gap between the leaflets 20, 22 when the device 402 is attached to the leaflets.

The coaptation element 3800 can be disposed around one or more of the shafts or other control elements of the system 400. For example, the coaptation element 3800 can be disposed around the shaft 403, the shaft 413, the paddle control mechanism 410, and/or the lock control mechanism 412.

The device 402 can include any other features for a device, treatment device, repair device, implant, etc. discussed in the present application, and the device 402 can be positioned to engage valve tissue as part of any suitable treatment and/or repair system (e.g., any treatment and/or repair system disclosed in the present application, or the applications cited herein). Additional features of the device 402, modified versions of the device, delivery systems for the device, and methods for using the device and delivery system are disclosed by Patent Cooperation Treaty International Application No. PCT/US2019/012707 (International Publication No. WO 2019139904). Any combination or sub-combination of the features disclosed by the present application can be combined with any combination or sub-combination of the features disclosed by Patent Cooperation Treaty International Application No. PCT/US2019/012707 (International Publication No. WO 2019139904).

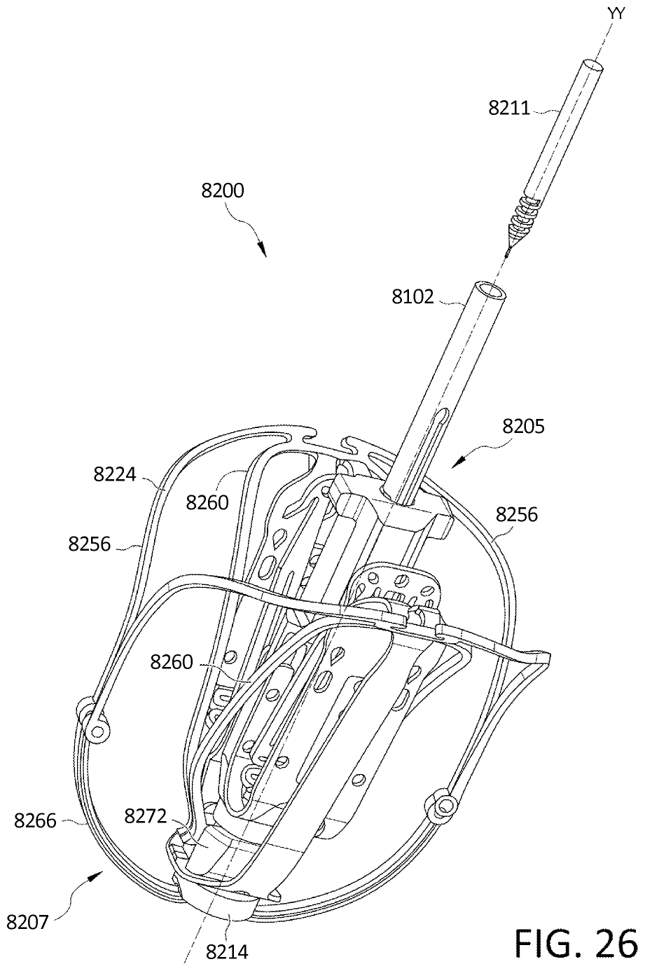

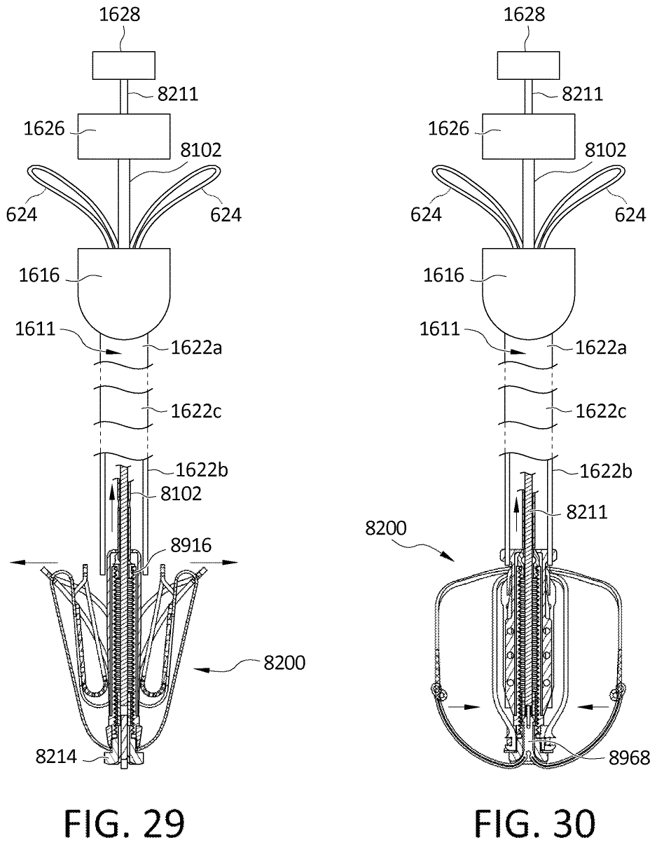

FIGS. 26-30 illustrate an example of one of the many systems for treating and/or repairing a native valve of a patient that the concepts of the present application can be applied to. Referring to FIGS. 29 and 30, the system includes a catheter assembly 1611 (e.g., a device catheter assembly, an implant catheter assembly, treatment catheter assembly, etc.) and a treatment and/or repair device 8200. Referring to FIGS. 26-28, the device 8200 includes a proximal or attachment portion 8205, paddle frames 8224, and a distal portion 8207. The attachment portion 8205, the distal portion 8207, and the paddle frames 8224 can be configured in a variety of ways.

In the example illustrated in FIG. 26, the paddle frames 8224 can be symmetric along longitudinal axis YY. However, in some implementations, the paddle frames 8224 are not symmetric about the axis YY. Moreover, referring to FIG. 26, the paddle frames 8224 include outer frame portions 8256 and inner frame portions 8260.

In some implementations, the connector 8266 (e.g., shaped metal component, shaped plastic component, tether, wire, strut, line, cord, suture, etc.) attaches to the outer frame portions 8256 at outer ends of the connector 8266 and to a coupler 8972 at an inner end 8968 of the connector 8266 (see FIG. 28). Between the connector 8266 and the attachment portion 8205, the outer frame portions 8256 form a curved shape. For example, in the illustrated example, the shape of the outer frame portions 8256 resembles an apple shape in which the outer frame portions 8256 are wider toward the attachment portion 8205 and narrower toward the distal portion 8207. In some implementations, however, the outer frame portions 8256 can be otherwise shaped.

The inner frame portions 8260 extend from the attachment portion 8205 toward the distal portion 8207. The inner frame portions 8260 then extend inward to form retaining portions 8272 that are attached to the actuation cap 8214. The retaining portions 8272 and the actuation cap 8214 can be configured to attach in any suitable manner.

In some implementations, the inner frame portions 8260 are rigid frame portions, while the outer frame portions 8256 are flexible frame portions. The proximal end of the outer frame portions 8256 connect to the proximal end of the inner frame portions 8260, as illustrated in FIG. 26.

The width adjustment element 8211 (e.g., width adjustment wire, width adjustment shaft, width adjustment tube, width adjustment line, width adjustment cord, width adjustment suture, width adjustment screw or bolt, etc.) is configured to move the outer frame portions 8256 from the expanded position to the narrowed position by pulling the inner end 8968 (FIG. 28) and portions of the connector 8266 into the actuation cap 8214. The actuation element 8102 is configured to move the inner frame portions 8260 to open and close the paddles in accordance with some implementations disclosed herein.

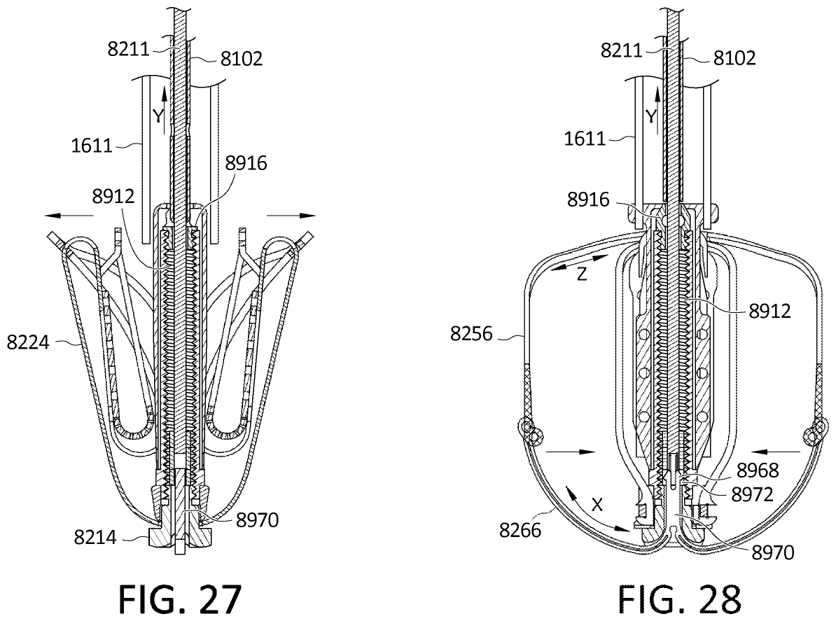

As shown in FIGS. 27 and 28, the connector 8266 has an inner end 8968 that engages with the width adjustment element 8211 such that a user can move the inner end 8968 inside the receiver 8912 (e.g., an internally threaded element, a column, a conduit, a hollow member, a notched receiving portion, a tube, a shaft, a sleeve, a post, a housing, a cylinder, tracks, etc.) to move the outer frame portions 8256 between a narrowed position and an expanded position. In the illustrated example, the inner end 8968 includes a post 8970 that attaches to the outer frame portions 8256 and a coupler 8972 that extends from the post 8970. The coupler 8972 is configured to attach and detach from both the width adjustment element 8211 and the receiver 8912. The coupler 8972 can take a wide variety of different forms. For example, the coupler 8972 can include one or more of a threaded connection, features that mate with threads, detent connections, such as outwardly biased arms, walls or other portions. When the coupler 8972 is attached to the width adjustment element 8211, the coupler is released from the receiver 8912. When the coupler 8972 is detached from the width adjustment element 8211, the coupler is secured to the receiver. The inner end 8968 of the connector can, however, be configured in a variety of ways. Any configuration that can suitably attach the outer frame portions 8256 to the coupler to allow the width adjustment element 8211 to move the outer frame portions 8256 between the narrowed position and the expanded position can be used. The coupler can be configured in a variety of ways as well and can be a separate component or be integral with another portion of the device, e.g., of the connector or inner end of the connector.

The width adjustment element 8211 allows a user to expand or contract the outer frame portions 8256 of the device 8200. In the example illustrated in FIGS. 27 and 28, the width adjustment element 8211 includes an externally threaded end that is threaded into the coupler 8972. The width adjustment element 8211 moves the coupler in the receiver 8912 to adjust the width of the outer frame portions 8256. When the width adjustment element 8211 is unscrewed from the coupler 8972, the coupler engages the inner surface of the receiver 8912 to set the width of the outer frame portions 8256.

In some implementations, the receiver 8912 can be integrally formed with a distal cap 8214. Moving the cap 8214 relative to a body of the attachment portion 8205 opens and closes the paddles. In the illustrated example, the receiver 8912 slides inside the body of the attachment portion. When the coupler 8972 is detached from the width adjustment element 8211, the width of the outer frame portions 8256 is fixed while the actuation element 8102 moves the receiver 8912 and cap 8214 relative to a body of the attachment portion 8205. Movement of the cap can open and close the device in the same manner as some of the examples disclosed above.

In the illustrated example, a driver head 8916 is disposed at a proximal end of the actuation element 8102. The driver head 8916 releasably couples the actuation element 8102 to the receiver 8912. In the illustrated example, the width adjustment element 8211 extends through the actuation element 8102. The actuation element is axially advanced in the direction opposite to direction Y to move the distal cap 8214. Movement of the distal cap 8214 relative to the attachment portion 8205 is effective to open and close the paddles, as indicated by the arrows in FIG. 27. That is, movement of the distal cap 8214 in the direction Y closes the device and movement of the distal cap in the direction opposite to direction Y opens the device.

Also illustrated in FIGS. 27 and 28, the width adjustment element 8211 extends through the actuation element 8102, the driver head 8916, and the receiver 8912 to engage the coupler 8972 attached to the inner end 8968. The movement of the outer frame portions 8256 to the narrowed position can allow the device or implant 8200 to maneuver more easily into position for deployment and/or implantation in the heart by reducing the contact and/or friction between the native structures of the heart—e.g., chordae—and the device 8200. The movement of the outer frame portions 8256 to the expanded position provides the anchor portion of the device 8200 with a larger surface area to engage and capture leaflet(s) of a native heart valve.

Referring to FIGS. 29 and 30, an example of a catheter assembly 1611 (e.g., a device catheter assembly, an implant catheter assembly, treatment catheter assembly, etc.) in which clasp actuation lines 624 extend through a handle 1616, the actuation element 8102 is coupled to a paddle actuation control 1626, and the width adjustment element 8211 is coupled to a paddle width control 1628. A proximal end portion 1622a of the shaft or catheter of the catheter assembly 1611 can be coupled to the handle 1616, and a distal end portion 1622b of the shaft or catheter can be coupled to the device 8200. The actuation element 8102 can extend distally from the paddle actuation control 1626, through the handle 1616, through the delivery shaft or catheter of the catheter assembly 1611, and through the proximal end of the device 8200, where it couples with the driver head 8916. The actuation element 8102 can be axially movable relative to the outer shaft of the catheter assembly 1611 and the handle 1616 to open and close the device.

The width adjustment element 8211 can extend distally from the paddle width control 1628, through the paddle actuation control 1626 and through the actuation element 8102 (and, consequently, through the handle 1616, the outer shaft of the implant catheter assembly 1611, and through the device 8200), where it couples with the movable coupler 8972. The width adjustment element 8211 can be axially movable relative to the actuation element 8102, the outer shaft of the implant catheter assembly 1611, and the handle 1616. The clasp actuation lines 624 can extend through and be axially movable relative to the handle 1616 and the outer shaft of the implant catheter assembly 1611. The clasp actuation lines 624 can also be axially movable relative to the actuation element 8102.

Referring to FIGS. 29 and 30, the width adjustment element 8211 can be releasably coupled to the coupler 8972 of the device 8200. Advancing and retracting the width adjustment element 8211 with the paddle width control 1628 widens and narrows the paddles. Advancing and retracting the actuation element 8102 with the paddle actuation control 1626 opens and closes the paddles of the device.

In the examples of FIGS. 29 and 30, the catheter or shaft of the catheter assembly 1611 is an elongate shaft extending axially between the proximal end portion 1622a, which is coupled to the handle 1616, and the distal end portion 1622b, which is coupled to the device 8200. The outer shaft of the catheter assembly 1611 can also include an intermediate portion 1622c disposed between the proximal and distal end portions 1622a, 1622b.

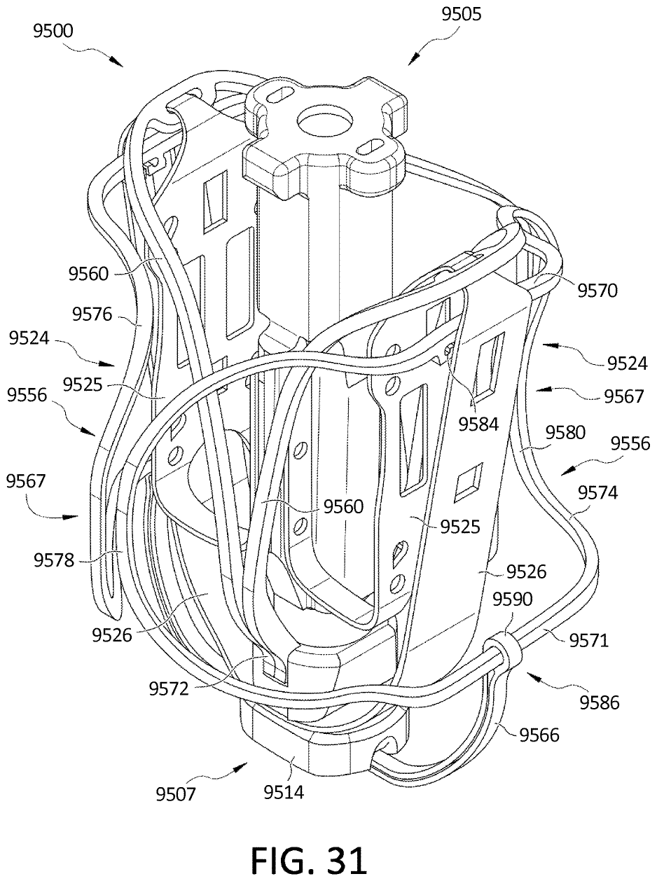

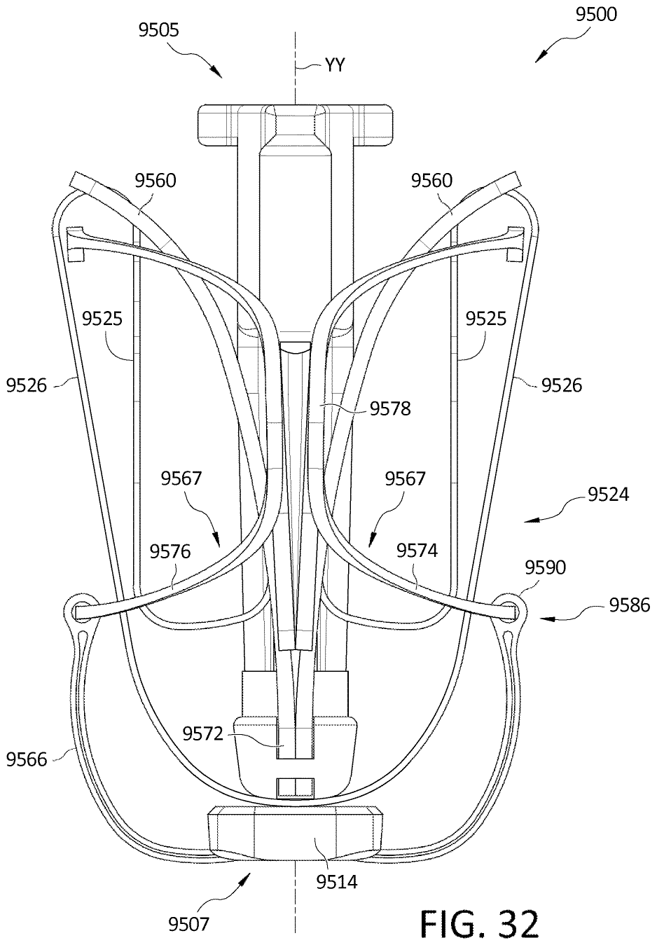

FIGS. 31-33 illustrate an example of one of the many systems for treating and/or repairing a native valve of a patient that the concepts of the present application can be applied to. The system includes a treatment and/or repair device 9500 (e.g., a treatment device, a repair device, a valve repair device, a valve treatment device, a prosthetic device, an implantable device, an implant, etc.) having a proximal or attachment portion 9505, paddle frames 9524, inner paddles 9525, outer paddles 9526, and a distal portion 9507. The proximal portion 9505, the distal portion 9507, the paddle frames 9524, and the paddles 9525, 9526 can be configured in a variety of ways, such as any configuration disclosed herein. While examples with pairs of paddles are described, other numbers of paddles are possible (e.g., one, three, etc.).

The example device 9500 illustrated in FIGS. 31-33 can be the same as or similar to the device 9200 of FIG. 26. For example, the paddle frames 9524 can be symmetric along a longitudinal axis YY (FIG. 26). However, in some implementations, the paddle frames 9524 are not symmetric about the longitudinal axis YY. The paddle frames 9524 include an outer frame member or members 9556 (e.g., wire frame portion, metal frame portion, etc.) and an inner frame member or members 9560 (e.g., wire frame portion, metal frame portion, etc.).

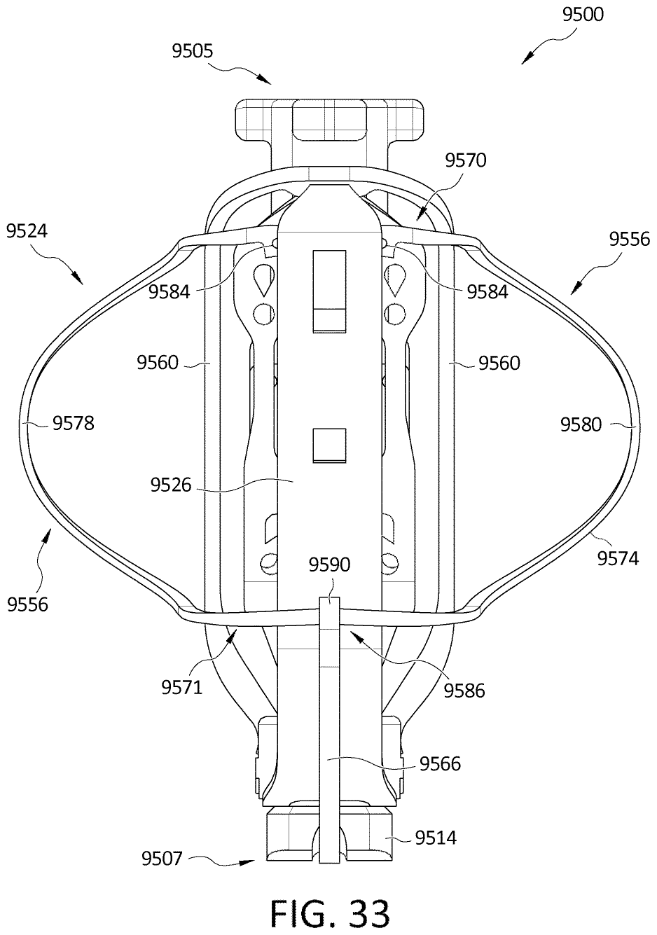

The outer frame members 9556 are flexibly attached to a connector 9566 at the distal portion 9507. Between the connector 9566 and the proximal portion 9505, the outer frame members 9556 form a curved shape. For example, in the illustrated example, the shape of the outer frame members 9556 resembles an oval shape as shown in FIG. 33. In some implementations, however, the outer frame members 9556 can be otherwise shaped.

The inner frame members 9560 extend from the proximal portion 9505 toward the distal portion 9507. The inner frame members 9560 then extend inward to form retaining portions 9572 that are attached to an actuation cap 9514, in the same or similar manner as described in relation to any of the device implementations described herein. The retaining portions 9572 and the actuation cap 9514 can be configured to attach in any suitable manner.

In some implementations, the inner frame members 9560 are rigid frame portions, while the outer frame members 9556 are flexible frame portions. The proximal end of the outer frame members 9556 can connect to the proximal end of the inner frame members 9560 at, or adjacent, the proximal portion 9505 of the device. In some implementations, as shown in FIGS. 31-33, the proximal ends of the outer frame members 9556 and the proximal ends of the inner frame members 9560 are positioned to connect to the paddles 9525, 9526 at locations spaced apart from each other.

The implantable device 9500 is configured to move the outer frame members 9556 from an expanded position to a narrowed position by pulling portions of the connector 9566 into the actuation cap 9514. The implantable device 9500 is also configured to move the inner frame members 9560 to open and close the paddles 9525, 9526 in accordance with some implementations disclosed herein.

In the example device 9500 of FIGS. 31-34, the device 9500 includes the pair of adjustable width frame members 9567, one arranged anteriorly and the other arranged posteriorly. In particular, the device 9500 includes an anterior adjustable width frame member 9574 and a posterior adjustable width frame member 9576, both of which connect to a connector 9566. In the device 9500, the connector 9566 is arranged to extend anteriorly and posteriorly as opposed to medially and laterally as with the device 8200 of FIGS. 26-30.

The anterior adjustable width frame member 9574 and the posterior adjustable width frame member 9576 can be formed in any suitable manner. For example, anterior adjustable width frame member 9574 and the posterior adjustable width frame member 9576 can include a shape-memory alloy, such as Nitinol, to provide shape-setting capability. Each of the anterior adjustable width frame member 9574 and the posterior adjustable width frame member 9576 can be shape-set into the as-assembled condition or assembled in a flat condition or profile. In the illustrated example, the anterior adjustable width frame member 9574 and the posterior adjustable width frame member 9576 are identical. Thus, the description below of the anterior adjustable width frame member 9574 applies equally to the posterior adjustable width frame member 9576. In some implementations, however, the anterior adjustable width frame member 9574 and the posterior adjustable width frame member 9576 can be configured differently.

The anterior adjustable width frame member 9574 (e.g., wire frame portion, metal frame portion, etc.) can be configured in a variety of ways, including different shapes and sizes. Referring to FIGS. 31-33, in the illustrated example, the anterior adjustable width frame member 9574 is formed as a closed loop having a proximal portion 9570, a distal portion 9571 opposite the proximal portion 9570, a medial portion 9578 extending between the proximal portion 9570 and the distal portion 9571, and a lateral portion 9580 extending between the proximal portion 9570 and the distal portion 9571 and opposite the medial portion 9578. In some implementations, however, the anterior adjustable width frame member 9574 can be open-ended, such as for example at the proximal portion 9570.

The proximal portion 9570 of the anterior adjustable width frame member 9574 can also include one or more recesses or holes 9584 (FIGS. 31 and 33) for receiving a portion of one of the paddles (e.g., the inner and outer paddles). The distal portion 9571 is connected to a proximal portion 9586 of the connector 9566 at, or adjacent, the midplane of the device 9500. The distal portion 9571 of the anterior adjustable width frame member 9574 can connect to the proximal portion 9586 of the connector 9568 in a variety of ways. In the illustrated example, the distal portion 9571 is a linear segment extending across the device 9500 between a medial side and a lateral side.

The connector 9566 includes a proximal portion 9586 adjacent the distal portion 9571 of the anterior adjustable width frame member 9574. The proximal portion 9586 includes an attachment portion 9590 for connecting to the distal portion 9571. The attachment portion 9590 can be configured in a variety of ways. In the illustrated example, the attachment portion 9590 includes a loop or hole through which the distal portion 9571 passes through.

The connector 9566 can include one, two, or more layers of material. To connect the distal portion 9571 to the attachment portions 9590, the layers can be separated to temporarily open the attachment portion 9590 at the proximal portion 9586 to accept the distal portion 9571. Once the distal portion 9571 is properly positioned, the layers can be allowed to move back together to encircle the distal portion 9571. The illustrated material bridge and resulting closed hole is optional.

In the example FIGS. 31-33, when the distal portion 9571 of the anterior adjustable width frame member 9574 is attached to the connector 9566 by the attachment portion 9590 at the proximal portion 9586, the distal portion 9571 is positioned outward of the outer paddle 9526 (i.e., further anteriorly). In some implementations, however, the distal portion 9571 can be positioned inward of the outer paddle 9526 (i.e., located less anteriorly than the outer paddle 9526).

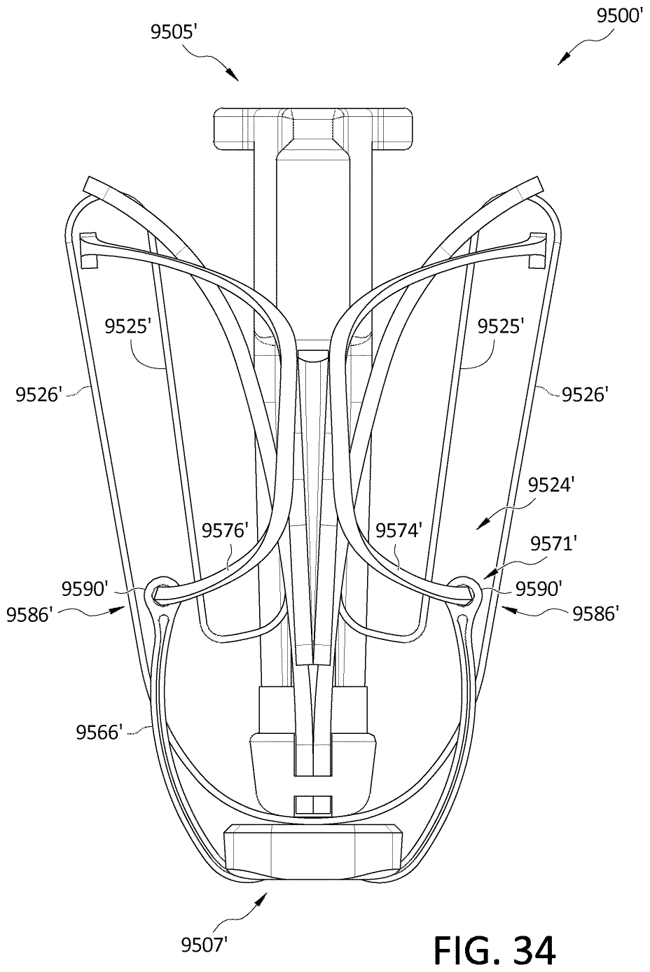

For example, FIG. 34 illustrates an example of a device 9500′ (e.g., a treatment device, a repair device, a valve repair device, a valve treatment device, a prosthetic device, an implantable device, an implant, etc.) having a proximal or attachment portion 9505′, paddle frames 9524′, inner paddles 9525′, outer paddles 9526′, and a distal portion 9507′. The device 9500′ can be configured substantially the same as the device 9500 of FIGS. 31-33. Thus, the description of the device 9500 applies equally to the device 9500′. For the device 9500′, however, when the distal portions 9571′ of the adjustable width frame members 9574′, 9576′ are attached to the proximal portions 9586′ of the connector 9566′ by the attachment portion 9590′, the distal portion 9571′ is positioned between the inner paddle 9525′ and the outer paddle 9526′ (i.e., to the anterior side of the inner paddle 9525′ and to the lateral side of the outer paddle 9526). The connector 9566′ extends through an opening or cutout in the outer paddle 9526′. The connection portion 9590′ is also positioned between the inner paddle 9525′ and the outer paddle 9526′ (i.e., to the anterior side of the inner paddle 9525′ and to the lateral side of the outer paddle 9526). As shown in FIG. 34., the distance between the connectors 9566′ is less (i.e., a narrower spread) than the distance between the connecting portions 9566 of the device 9500 of FIGS. 31-33 (i.e., a wider spread).

Referring to FIGS. 31-33, the posterior adjustable width frame member 9576 can connect to the connector 9566 in the same manner as the anterior adjustable width frame member 9574. In some implementations, the attachment points between the adjustable width frame member 9567 and the connector 9566 act as hinges allowing for free rotation or pivoting between the adjustable width frame member 9567 and connector 9566 at the attachment point resulting in reduced stress and tension on the delivery system and device. Further, by arranging the attachment points between the adjustable width frame and the distal outer frame anteriorly and posteriorly, rather than medially and laterally, the attachment points do not restrict the depth to which the mitral valve leaflets can be inserted into the paddles. Thus, the leaflets can be fully received into the paddles.

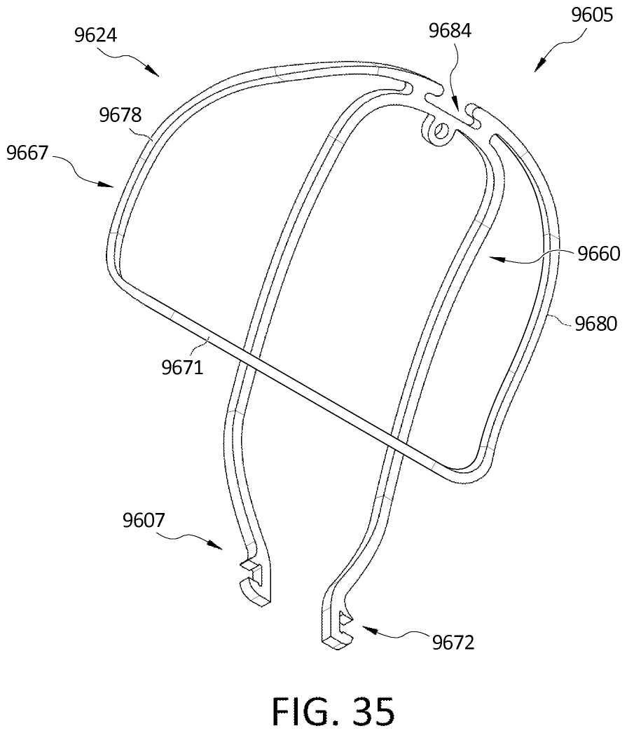

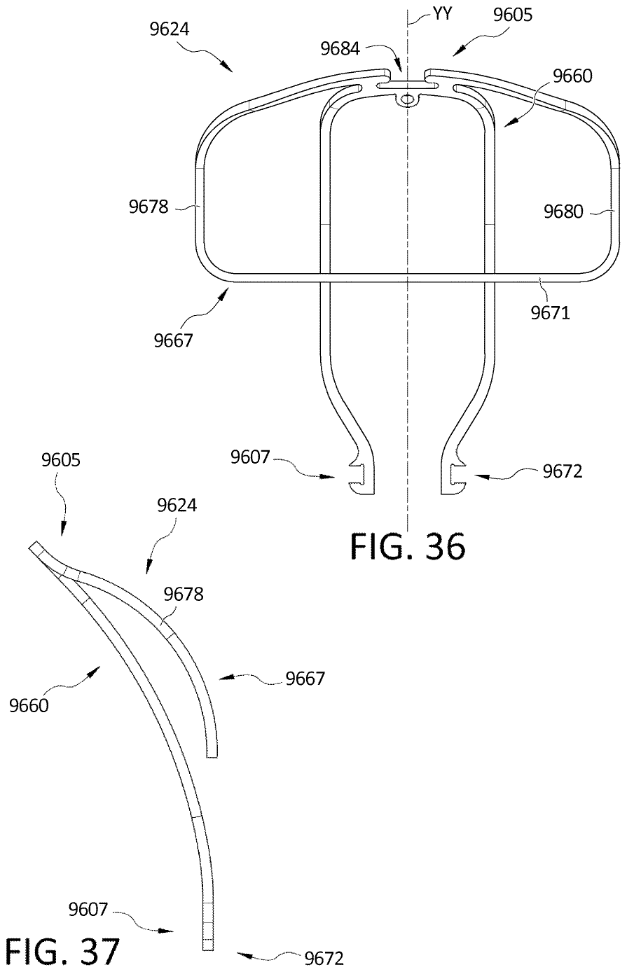

FIGS. 35-37 illustrate an example of a paddle frame 9624 that can be used with various systems (e.g., a treatment system, a repair system, a valve repair system, a valve treatment system, etc.) and devices (e.g., device 9500, other systems and/or devices herein, etc.) usable for treating and/or repairing a native valve of a patient that the concepts of the present application can be applied to.

In some implementations, the paddle frame 9624 includes an adjustable width frame member or members 9667 and an inner frame member or members 9660 that are formed as a single, integral piece. In some implementations, the adjustable width frame member or members 9667 connects to a lower or distal frame member (e.g., connector 9566 of FIG. 31).

The paddle frame 9624 can optionally be formed as a single, integral piece in any suitable manner. For example, the paddle frame 9624 can include a shape-memory alloy, such as Nitinol, to provide shape-setting capability. The paddle frame 9624 can be shape-set into the as-assembled condition or assembled in a flat condition or profile. The paddle frame 9624 can be symmetric along a longitudinal axis YY (FIG. 36). However, in some implementations, the paddle frames 9624 are not symmetric about the longitudinal axis YY. The paddle frame 9624 includes a proximal portion 9605 and a distal portion 9607 opposite the proximal portion 9605.

In some implementations, the inner frame members 9660 (e.g., wire frame portion, metal frame portion, etc.) extend from the proximal portion 9605 toward the distal portion 9607. In some implementations, the inner frame members 9660 at the distal portion 9607 extend inward to form retaining portions 9672 that are attached to an actuation cap (e.g., the actuation cap 9514 of FIG. 31), in the same or similar manner as described in relation to any of the example device described herein. The retaining portions 9672 can be configured to attach in any suitable manner.

In some implementations, the inner frame members 9660 are rigid frame portions, while the adjustable width frame members 9667 are flexible frame portions. In some implementations, the adjustable width frame members 9667 can integrally connect to the inner frame members 9660 at, or adjacent, the proximal portion 9605 of the paddle frame 9624. In some implementations, the proximal portion 9605 can also include one or more recesses or holes 9684 (FIGS. 35-36) for receiving a portion of one of the paddles (e.g., the inner and outer paddles).

In some implementations, the adjustable width frame members 9667 are flexibly attached to connection portions of a connector (e.g., the connection portions 9566 of the connector 9568 of FIGS. 31-33). The adjustable width frame member 9667 can be configured in a variety of ways, including different shapes and sizes. In the illustrated example, the adjustable width frame member 9667 is formed as a closed loop having a distal portion 9671 opposite the proximal portion 9605, a medial portion 9678 extending between the proximal portion 9505 and the distal portion 9671, and a lateral portion 9680 extending between the proximal portion 9605 and the distal portion 9671 and opposite the medial portion 9678.

In some implementations, the distal portion 9671 is configured to connect to a proximal end of a connector (e.g., the proximal portion 9586 of the connector 9566 of FIGS. 31-33) at, or adjacent, the midplane of the device. The distal portion 9671 can connect to the proximal end of the connector in a variety of ways. In the illustrated example, the distal portion 9671 is a linear segment extending across the device between a medial side and a lateral side and is configured to be received through a loop or hole in a connection portion in the same manner as the device of FIGS. 31-33.

Between the proximal portion 9605 and the distal portion 9671, the adjustable width frame members 9667 form a curved shape. For example, in the view illustrated in FIG. 35, the shape of the adjustable width frame members 9667 resembles a rectangle with the proximal portion 9605 curved or domed. In some implementations, however, the adjustable width frame members 9667 can be otherwise shaped.

In some implementations, the device (e.g., a treatment device, a repair device, a valve repair device, a valve treatment device, a prosthetic device, an implantable device, an implant, etc.) is configured to move one or more adjustable width frame members 9667 from an expanded position to a narrowed position by pulling portions of the connector into the actuation cap in the same manner as the connector 9566 and actuation cap 9514 of FIG. 31. The device is also configured to move the one or more inner frame members 9660 to open and close the one or more paddles (e.g., outer paddles 320 and inner paddles 322 of FIG. 23) in accordance with some implementations disclosed herein.

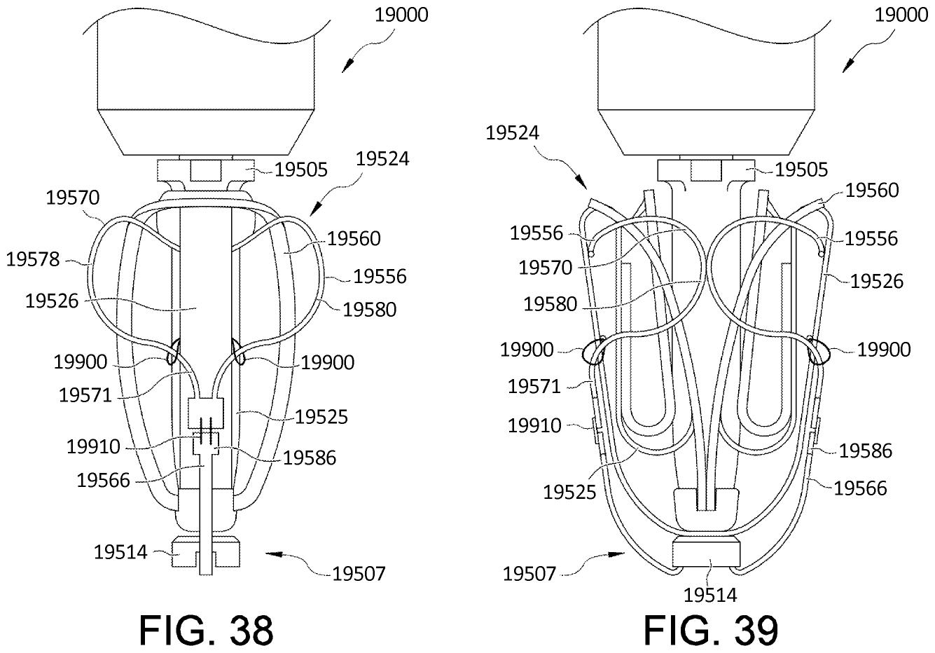

FIGS. 38-41 illustrate an example of one of the many systems (e.g., a treatment system, a repair system, a valve repair system, a valve treatment system, etc.) useable for treating and/or repairing a native valve of a patient that the concepts of the present application can be applied to.

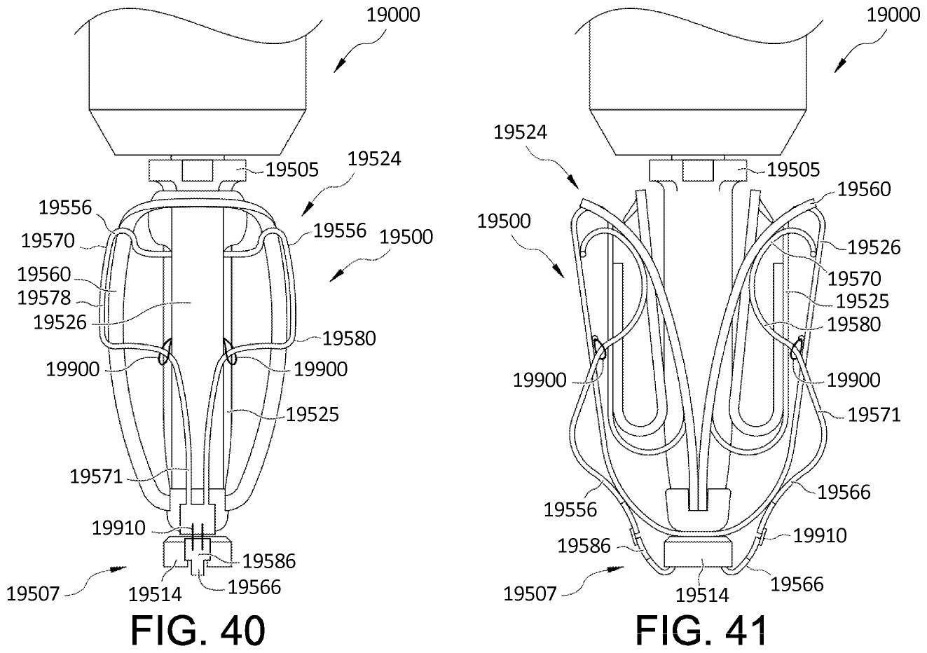

In some implementations, the device 19500 (e.g., a treatment device, a repair device, a valve repair device, a valve treatment device, a prosthetic device, an implantable device, an implant, etc.) includes one or more guides 19900 that are configured to guide or control the shape of each outer frame member 19556 as the outer frame member is moved between the expanded configuration (FIGS. 38 and 39) and the narrow configuration (FIGS. 40 and 41). The guide 19900 can take a variety of different forms. For example, the guide 19900 can comprise one or more of a loop, a ring, a cutout, a slot, a sling, a passage, a channel, etc. In some implementations, one or more portions of the outer frame member 19556 extend through the guide.

In some implementations, the guide 19900 acts as a choke point. In some implementations, a proximal portion 19570 of the outer frame member 19556 is proximal to the guide 19900 and a distal portion 19571 of the outer frame member 19556 is distal to the guide 19900. In some implementations, the proximal portion 19570 of the outer frame member 19556 can expand and contract. In some implementations, the guide 19900 pulls or chokes a medial portion 19578 and a lateral portion 19580 of the outer frame member 19556 together when viewed from a side of the device (see FIGS. 38 and 40). In some implementations, the two outer frame members 19556 move apart or separate as the outer frame members are moved from the expanded configuration to the narrow configuration (see FIGS. 39 and 41). In some implementations, the medial portion 19578 and the lateral portion 19580 of the outer frame member 19556 are shaped and/or configured such that the two outer frame members 19556 stay in contact with one another or stay close to one another as the outer frame members are moved from the expanded configuration to the narrow configuration.

In some implementations, the distal portion 19571 comprises portions of the medial portion 19578 and a lateral portion 19580 that are distal to the guide 19900. In some implementations, the medial portion 19578 and a lateral portion 19580 that are distal to the guide 19900 are close together. In some implementations, the medial portion 19578 and a lateral portion 19580 that are proximal to the guide 19900 move toward and away from one another (when viewed from the side—e.g., FIGS. 38 and 40) to change the width of the outer frame member 19556.

In some implementations, the system 19000 (e.g., treatment system, repair system, valve repair system, valve treatment system, etc.) includes a device 19500 (e.g., a treatment device, a repair device, a valve repair device, a valve treatment device, a prosthetic device, an implantable device, an implant, etc.) having a proximal or attachment portion 19505, one or more paddle frames 19524, one or more inner paddles 19525, one or more outer paddles 19526, and a distal portion 19507. The proximal portion 19505, the distal portion 19507, the one or more paddle frames 19524, and the one or more paddles 19525, 19526 can be configured in a variety of ways, such as any configuration disclosed herein.

The example device 19500 illustrated in FIGS. 38-41 can be the same as or similar to the device 8200 of FIG. 26. For example, the paddle frames can be symmetric along a longitudinal axis YY (FIG. 26). However, in some implementations, the paddle frames are not symmetric about the longitudinal axis YY. The paddle frames include an outer frame member or members 19556 (e.g., wire frame portion, metal frame portion, etc.) and an inner frame member or members 19560 (e.g., wire frame portion, metal frame portion, etc.). The outer frame members 19556 are flexibly attached to a connector 19566 at the distal portion 19507. Between the connector 19566 and the proximal portion 19505, the outer frame members 19556 optionally form a curved shape.

In some implementations, the inner frame members 19560 extend from the proximal portion 19505 toward the distal portion 19507. In some implementations, the inner frame members 19560 then extend inward to form retaining portions that are attached to an actuation cap 19514, in the same or similar manner as described in relation to any of the example devices described herein. The retaining portions and the actuation cap 19514 can be configured to attach in any suitable manner.

In some implementations, the inner frame members 19560 are rigid frame portions, while the outer frame members 19556 are flexible frame portions. The proximal end of the outer frame members 19556 can connect to the proximal end of the inner frame members 19560 at, or adjacent, the proximal portion 19505 of the device.

In some implementations, the treatment and/or repair device 19500 is configured to move the outer frame members 19556 from an expanded position to a narrowed position (FIGS. 40 and 41) by pulling portions of the connector 19566 into the actuation cap 19514. In some implementations, the device 19500 is also configured to move the inner frame members 19560 to open and close the paddles 19525, 19526 in accordance with some implementations disclosed herein.

In some implementations, as in the example device 19500 of FIGS. 38-41, the device 19500 includes the pair of adjustable width frame members 19556, one arranged anteriorly and the other arranged posteriorly. In particular, the illustrated example device 19500 includes an anterior adjustable width frame member and a posterior adjustable width frame member, both of which connect to a connector 19566. In the device 19500, the connector 19566 is arranged to extend anteriorly and posteriorly as opposed to medially and laterally as with the device 8200 of FIGS. 26-30. However, in some implementations, the connector 19566 can be arranged to extend medially and laterally in the same or similar manner as with the device 8200 of FIGS. 26-30.

The anterior adjustable width frame member and the posterior adjustable width frame member can be formed in any suitable manner. For example, the anterior adjustable width frame member and the posterior adjustable width frame member can include a shape-memory alloy, such as Nitinol, to provide shape-setting capability. Each of the anterior adjustable width frame member and the posterior adjustable width frame member can be shape-set into the as-assembled condition or assembled in a flat condition or profile. In the illustrated example, the anterior adjustable width frame member and the posterior adjustable width frame member are identical. Thus, the description below of the anterior adjustable width frame member applies equally to the posterior adjustable width frame member. In some implementations, however, the anterior adjustable width frame member and the posterior adjustable width frame member can be configured differently.

The anterior adjustable width frame member (e.g., wire frame portion, metal frame portion, etc.) can be configured in a variety of ways, including different shapes and sizes. Referring to FIGS. 38-41, in the illustrated example, the anterior adjustable width frame member is formed as a closed loop having a proximal portion 19570, a distal portion 19571 opposite the proximal portion 19570, a medial portion 19578 extending between the proximal portion 19570 and the distal portion 19571, and a lateral portion 19580 extending between the proximal portion 19570 and the distal portion 19571 and opposite the medial portion 19578. In some implementations, however, the anterior adjustable width frame member can be open-ended, such as for example at the proximal portion 19570 and/or at the distal portion 19571.

In some implementations, the distal portion 19571 is connected to a proximal portion 19586 of the connector 19566 at, or adjacent, the midplane of the device 19500. The distal portion 19571 of the anterior adjustable width frame member can connect to the proximal portion 19586 of the connector 19566 in a variety of ways. In the illustrated example, the distal portion 19571 is a segment extending across the device 19500 between a medial side and a lateral side. In some implementations, the proximal portion 19586 is configured to connect to the distal portion 19571. In some implementations, the proximal portion 19586 accommodates sutures 19910 for attaching the distal portion 19571 to the proximal portion 19586.

Referring to FIGS. 38-41, the posterior adjustable width frame member can connect to the connector 19566 in the same manner as the anterior adjustable width frame member. The attachment points between the adjustable width frame member 19556 and the connector 19566 act as hinges optionally allowing for free rotation or pivoting between the adjustable width frame member 19556 and connector 19566 at the attachment point resulting in reduced stress and tension on the delivery system and device.

In some implementations, the device is configured to move the adjustable width frame member(s) from an expanded position to a narrowed position by pulling portions of the connector into the actuation cap in the same or similar manner as the connector 19566 and actuation cap 19514 of FIG. 31. In some implementations, the device is also configured to move the inner frame members 19560 to open and close the paddles (e.g., outer paddles 320 and inner paddles 322 of FIG. 23) in accordance with some implementations disclosed herein.

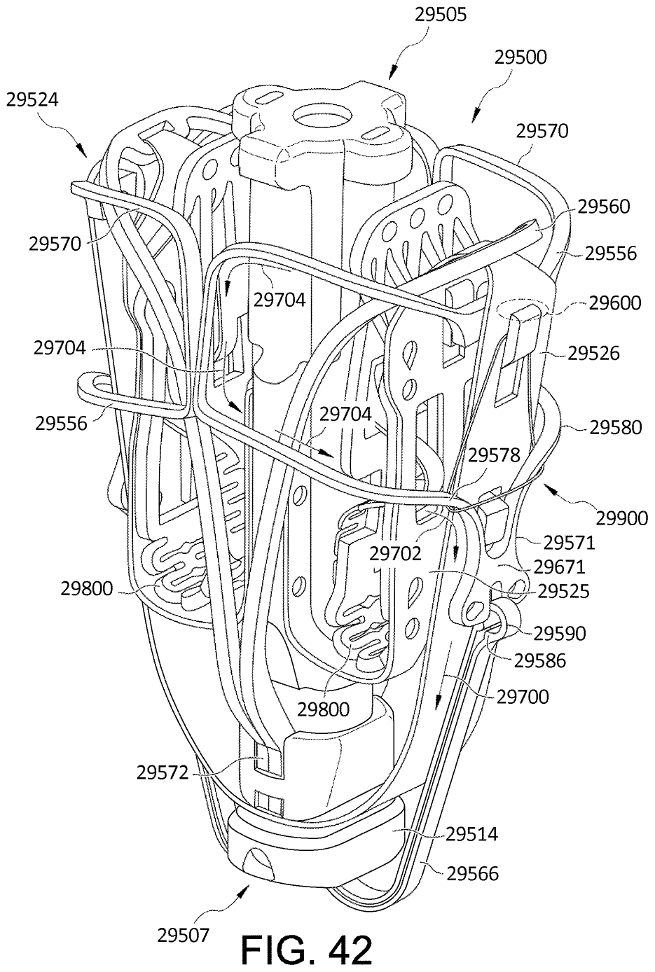

FIGS. 42-44 illustrate three different examples of devices 29500, 39500, 49500 (e.g., treatment devices, repair devices, valve repair devices, valve treatment devices, prosthetic devices, implantable devices, implants, etc.) having different guides 29900, 39900, 49900 and different outer frame members 29556, 39556, 49556. Other than the guides 29900, 39900, 49900 and the outer frame members 29556, 39556, 49556, the devices 29500, 39500, 49500 can be the same or substantially the same. In some implementations, the devices 29500, 39500, 49500 each include one or more guides 29900, 39900, 49900 that are configured to guide or control the shape of each outer frame member 29556, 39556, 49556 as the outer frame member is moved between the expanded configuration and the narrow configuration. Each of the guides 29900, 39900, 49900 can take a variety of different forms. For example, each of the guides 29900, 39900, 49900 can comprise one or more of a loop, a ring, a cutout, a slot, a sling, a passage, a channel, etc. In some implementations, one or more portions of each of the outer frame members 29556, 39556, 49556 extend through the corresponding guide 29900, 39900, 49900.

In some implementations, each of the guides 29900, 39900, 49900 acts as a choke point. Referring to the example illustrated by FIG. 42, the guide 29900 is attached to an outer paddle 29526. In some implementations, the guide 29900 is a sling, such as the illustrated sling, but can take any form, such as a pair of loops, one or a pair of rings, a cutout or cutouts in the outer paddle 29526, a slot or slot in the outer paddle 29526, a passage, a channel, etc. In the example illustrated by FIG. 42, the guide 29900 is positioned such that the choke point is proximal to a free edge of a native valve leaflet. For example, the free edge of the native leaflet is expected to reach a hinge 29800 of a clasp of the device 29500 and the guide 29900 is above or proximal to the hinge 29800, so that the expanded outer frame member 29556 engages a portion of the native valve leaflet that is above (i.e., closer to the atrium) than the free edge of the native valve leaflet.

The adjustable width frame members 29556 (e.g., wire frame portion, metal frame portion, etc.) can be configured in a variety of ways, including different shapes and sizes. Referring to FIG. 42, in the illustrated example, the adjustable width frame members 29556 are formed as a closed loop having a proximal portion 29570, a distal portion 29571, a medial portion 29578 extending between the proximal portion 29570 and the distal portion 29571, and a lateral portion 29580 extending between the proximal portion 29570 and the distal portion 29571 and opposite the medial portion 29578. A distal connection portion 29671, such as a strut, extension, projection, etc. can extend from the distal portion 29571. The connection portion 29671 can be connected to the connector 29566. In some implementations, the adjustable width frame member 29556 can be open-ended, such as for example at the proximal portion 29570 and/or at the distal portion 29571.

In some implementations, the proximal portion 29570 of the adjustable width frame member 29556 can also include one or more recesses or holes for receiving a portion of one of the paddles (e.g., the inner and outer paddles). In some implementations, the connection portion 29671 is connected to a proximal portion 29586 of the connector 29566 at, or adjacent, the midplane of the device 29500. In some implementations, the connection portion 29671 of the adjustable width frame member 29556 can connect to the proximal portion 29586 of the connector 29566 in a variety of ways. In the illustrated example, the connection portion 29671 is connected to the connector 29566 by a suture, a fastener, etc.

Still referring for FIG. 42, in some implementations, a proximal portion 29570 of the outer frame member 29556 is proximal to the guide 29900 and a distal portion 29571 of the outer frame member 29556 is distal to the guide 29900. In some implementations, the proximal portion 29570 of the outer frame member 29556 is fixed to another component of the device 29500. For example, the proximal portion 29570 of the outer frame member 29556 can be attached to the outer paddle 29526 at connection point 29600 or area as illustrated, to the inner frame member 19560, to the inner paddle 19525, etc.

Still referring to FIG. 42, in some implementations, the proximal portion 29570 of the outer frame member 29556 can expand and contract. In some implementations, the guide 29900 pulls or chokes a medial portion 29578 and a lateral portion 29580 of the outer frame member 29556 together when the outer frame member 29556 is moved from the expanded configuration toward the contracted or narrow configuration. In the example illustrated by FIG. 42, the connector 29566 is pulled down or distally as indicated by arrow 29700 to move the outer frame member 29556 from the expanded configuration toward the contracted or narrow configuration. In some implementations, the connector pulls portions of the medial portion 29578 and the lateral portion 29580 through the choke point created by the guide 29900 as indicated by arrow 29702 and the outer frame member 29556 deflects as indicated by arrows 29704 from the expanded configuration to the narrow configuration.

In some implementations, as can be seen in FIG. 42, the portion of the outer frame member 29556 that engages the native valve leaflet moves generally downward or distally (as indicated by arrows 29704) as the outer frame member 29556 deflects from the expanded configuration to the narrow configuration. As such, the outer frame member 29556 can pull the native valve leaflet further into the device as the outer frame member 29556 moves from the expanded configuration to the narrow configuration.

In the example illustrated by FIG. 42, the connector 29566 is moved up or proximally, opposite to the direction indicated by arrow 29700 to move the outer frame member 29556 from the contracted or narrow configuration toward the expanded configuration. The connector pushes portions of the medial portion 29578 and the lateral portion 29580 through the choke point created by the guide 29900, opposite the direction as indicated by arrow 29702. The outer frame member 29556 deflects in the opposite direction to that indicated by arrows 29704 from the narrow configuration toward the expanded configuration. Since the guide 29900 is positioned such that the choke point is proximal to or above a free edge of a native valve leaflet, the portion of the outer frame member 29556 that engages the native valve leaflet when the outer frame member is moved from the narrow configuration toward the expanded configuration is also above or proximal to the free edge of the native valve leaflet.

In some implementations, the medial portion 29578 and the lateral portion 29580 are configured such that the two outer frame members 29556 move apart or separate as the outer frame members are moved from the expanded configuration to the narrow configuration. In some implementations, the medial portion 29578 and the lateral portion 29580 of the outer frame member 29556 are shaped and/or configured such that the two outer frame members 29556 stay in contact with one another or stay close to one another as the outer frame members are moved from the expanded configuration to the narrow configuration.

In some implementations, the distal portion 29571 comprises portions of the medial portion 29578 and a lateral portion 29580 that are distal to the guide 29900. In some implementations, the medial portion 29578 and a lateral portion 29580 that are distal to the guide 29900 are close together. In some implementations, the medial portion 29578 and a lateral portion 29580 that are proximal to the guide 29900 move toward and away from one another to change the width of the outer frame member 29556.

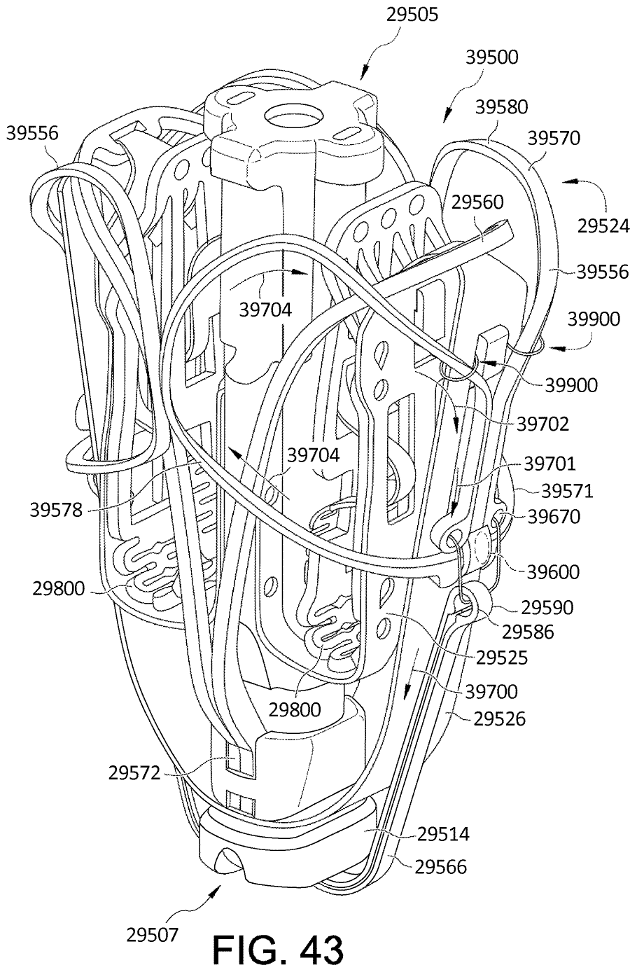

Referring to the example illustrated by FIG. 43, the guide 39900 is attached to an outer paddle 29526. In some implementations, the guide 39900 is a pair of loops, but can take any form, such as a sling, one or a pair of rings, a cutout or cutouts in the outer paddle 29526, a slot or slot in the outer paddle 29526, a passage, a channel, etc. In the example illustrated by FIG. 43, a portion of the outer frame member 39556 that engages the native valve leaflet is positioned proximal to a free edge of a native valve leaflet. For example, the free edge of the native leaflet is expected to reach a hinge 29800 of a clasp of the device 39500 and the expanded outer frame member 39556 engages a portion of the native valve leaflet that is above (i.e., closer to the atrium) than the free edge of the native valve leaflet.

The adjustable width frame members 39556 (e.g., wire frame portion, metal frame portion, etc.) can be configured in a variety of ways, including different shapes and sizes. Referring to FIG. 43, in the illustrated example, the adjustable width frame members 39556 are formed as an open loop having a proximal portion 39570, a distal portion 39571, a medial portion 39578 extending between the proximal portion 39570 and the distal portion 39571, and a lateral portion 39580 extending between the proximal portion 39570 and the distal portion 39571 and opposite the medial portion 39578. In some implementations, the proximal portion 39570 has the opening of the open loop. A proximal connection portion 39670, such as a strut, extension, projection, etc. can extend from the proximal portion 39570. The connection portion 39670 can be connected to the connector 29566. In some implementations, the adjustable width frame member 39556 can be a closed loop.

In some implementations, the distal portion 39571 of the adjustable width frame member 39556 can also include one or more recesses or holes for connection to one of the paddles (e.g., the inner and outer paddles). In some implementations, the connection portion 39670 is connected to the proximal portion 29586 of the connector 29566 at, or adjacent, the midplane of the device 29500. In some implementations, the connection portion 39670 of the adjustable width frame member 39556 can connect to the proximal portion 29586 of the connector 29566 in a variety of ways. In the illustrated example, the connection portion 29671 is connected to the connector 29566 by a suture, a fastener, etc.

Still referring for FIG. 43, in some implementations, a proximal portion 39570 of the outer frame member 39556 extends proximally to the guide 39900 and a distal portion 39571 of the outer frame member 39556 is distal to the guide 39900. In some implementations, the distal portion 39570 of the outer frame member 39556 is fixed to another component of the device 39500. For example, the distal portion 39571 of the outer frame member 39556 can be attached to the outer paddle 29526 at connection point 39600 or area as illustrated, or to another component of the device 39500.

Still referring to FIG. 43, in some implementations, the proximal portion 39570 of the outer frame member 39556 can expand and contract. In some implementations, the guide 39900 pulls or chokes a medial portion 39578 and a lateral portion 39580 of the outer frame member 39556 together when the outer frame member 39556 is moved from the expanded configuration toward the contracted or narrow configuration. In the example illustrated by FIG. 43, the connector 29566 is pulled down or distally as indicated by arrow 39700 to pull a portion of the outer frame member 39556 down or distally as indicated by arrow 39701. The downward or distal movement of the portion of the outer frame member 39556 indicated by arrow 39701 moves the outer frame member 39556 from the expanded configuration toward the contracted or narrow configuration.

In some implementations, the connector pulls upper or proximal portions of the medial portion 39578 and the lateral portion 39580 through the choke point created by the guide 39900 as indicated by arrow 39702 and the outer frame member 39556 deflects as indicated by arrows 39704 from the expanded configuration to the narrow configuration. In some implementations, the upper or proximal portions of the medial portion 39578 and the lateral portion 39580 are pulled past and/or over the connection point 39600 or area as the frame member 39556 deflects from the expanded configuration to the narrow configuration.

In some implementations, as can be seen in FIG. 43, the portion of the outer frame member 39556 that engages the native valve leaflet moves generally upward or proximally (as indicated by arrows 39704) as the outer frame member 39556 deflects from the expanded configuration to the narrow configuration.

In the example illustrated by FIG. 43, the connector 29566 is moved up or proximally, opposite to the direction indicated by arrow 39700 to move the outer frame member 39556 from the contracted or narrow configuration toward the expanded configuration. In some implementations, the connector pushes portions of the medial portion 39578 and the lateral portion 39580 through the choke point created by the guide 39900, opposite the direction as indicated by arrow 39702. In some implementations, the outer frame member 39556 deflects in the opposite direction to that indicated by arrows 39704 from the narrow configuration toward the expanded configuration. In some implementations, such as in the example illustrated by FIG. 43, the outer frame member 39556 can pull the native valve leaflet further into the device as the outer frame member 39556 moves from the expanded configuration to the narrow configuration.

In some implementations, the outer frame members 39556 move apart or separate as the outer frame members are moved from the expanded configuration to the narrow configuration. In some implementations, the medial portion 39578 and the lateral portion 39580 of the outer frame members 39556 are shaped and/or configured such that the two outer frame members 39556 stay in contact with one another or stay close to one another as the outer frame members are moved from the expanded configuration to the narrow configuration.

In some implementations, the medial portion 39578 and a lateral portion 39580 that are distal to the guide 39900 are close together (i.e., due to passing through the guide). In some implementations, the medial portion 39578 and a lateral portion 39580 that are on the other side of the guide 39900 move toward and away from one another to change the width of the outer frame member 39556.

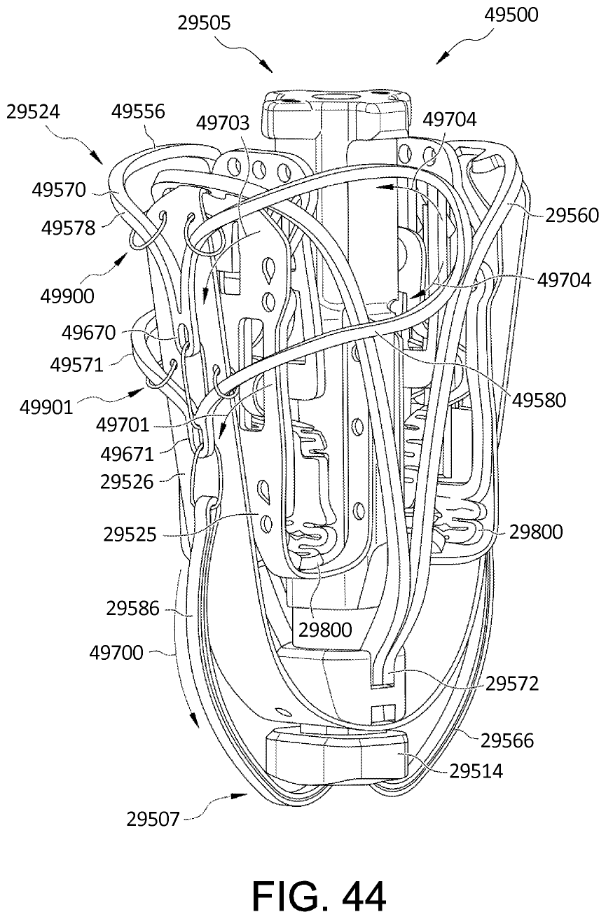

Referring to the example illustrated by FIG. 44, the device 49500 includes a proximal guide 49900 and a distal guide 49901. The proximal guide 49900 can be attached to an outer paddle 29526. In some implementations, the guide 49900 comprises a pair of loops, but can take any form, such as a sling, one or a pair of rings, a cutout or cutouts in the outer paddle 29526, a slot or slot in the outer paddle 29526, a passage, a channel, etc.

Still referring to FIG. 44, the distal guide 49901 can be attached to an outer paddle 29526. In some implementations, the distal guide 49901 is a pair of loops, but can take any form, such as a sling, one or a pair of rings, a cutout or cutouts in the outer paddle 29526, a slot or slot in the outer paddle 29526, a passage, a channel, etc. In the example illustrated by FIG. 44, a portion of the outer frame member 49556 that engages the native valve leaflet is positioned proximal to a free edge of a native valve leaflet. For example, the free edge of the native leaflet is expected to reach a hinge 29800 of a clasp of the device 49500 and the expanded outer frame member 49556 engages a portion of the native valve leaflet that is above (i.e., closer to the atrium) than the free edge of the native valve leaflet.

The adjustable width frame members 49556 (e.g., wire frame portion, metal frame portion, etc.) can be configured in a variety of ways, including different shapes and sizes. Referring to FIG. 44, in the illustrated example, the adjustable width frame members 49556 are formed as a closed loop having a proximal portion 49570, a distal portion 49571, a medial portion 49578 extending between the proximal portion 49570 and the distal portion 49571, and a lateral portion 49580 extending between the proximal portion 49570 and the distal portion 49571 and opposite the medial portion 49578. In some implementations, a proximal connection portion 49670, such as a strut, extension, projection, etc. can extend from the proximal portion 49570. A distal connection portion 49671, such as a strut, extension, projection, etc. can extend from the distal portion 49571. In some implementations, the connection portion 49671 can be connected to the connector 29566 and the connection potion 49670 can be connected to the connection portion 49671. In some implementations, the adjustable width frame member 49556 can be a closed loop as illustrated. In some implementations, the adjustable width frame member 49556 can be an open loop or can be two separate parts.

In some implementations, the connection portions 49670, 49671 can be at, or adjacent, the midplane of the device 29500. In some implementations, the connection portion 49671 of the adjustable width frame member 49556 can connect to the proximal portion 29586 of the connector 29566 in a variety of ways. In the illustrated example, the connection portion 49671 is connected to the connector 29566 by a suture, a fastener, etc. In some implementations, the proximal connection portion 49670 of the adjustable width frame member 49556 can connect to the distal connection portion 49671 in a variety of ways. In the illustrated example, the connection portion 49670 is connected to the connection portion 49671 by a suture, a fastener, etc.

Still referring for FIG. 44, in some implementations, a proximal portion 49570 of the outer frame member 49556 extends through the proximal guide 49900 and a distal portion 49571 of the outer frame member 39556 extends through the distal guide 49901. In some implementations, the outer frame member 49556 includes a proximal connection portion 49670 (e.g., strut, extension, connector, link, etc.) that extends from the proximal portion 49570 and a distal connection portion 49671 (e.g., strut, extension, connector, link, etc.) extends from the distal portion 49571. In some implementations, the proximal connection portion 49670 is connected to the distal connection portion 49671, for example, by a line, suture, link, connector, etc. The distal connection portion 49671 is connected to the connector 29566 for example, by a line, suture, link, connector, etc.

Still referring to FIG. 44, in some implementations, the outer frame member 49556 can expand and contract. In some implementations, the proximal guide 49900 and the distal guide 49901 each pull or choke a medial portion 49578 and a lateral portion 49580 of the outer frame member 49556 together when the outer frame member 49556 is moved from the expanded configuration toward the contracted or narrow configuration. In the example illustrated by FIG. 44, the connector 29566 is pulled down or distally as indicated by arrow 49700 to pull the distal connection portion 49671 of the outer frame member 49556 down or distally as indicated by arrow 49701. In turn, the distal connection portion 49671 pulls the proximal connection portion 49670 down or distally as indicated by arrow 49703. In some implementations, the downward or distal movement of the portion of the proximal connection portion 49670 and the distal connection portion 49671 moves the outer frame member 49556 from the expanded configuration toward the contracted or narrow configuration.

In some implementations, the connector pulls upper or proximal portions of the medial portion 49578 and the lateral portion 49580 through the choke point created by the proximal guide 49900. In some implementations, the connector also pulls lower or distal portions of the medial portion 49578 and the lateral portion 49580 through the choke point created by the distal guide 49901. The outer frame member 49556 deflects as indicated by arrows 49704 from the expanded configuration to the narrow configuration.

In some implementations, as can be seen in FIG. 44, the portion of the outer frame member 49556 that engages the native valve leaflet does not move upward or proximally or downwardly or distally as the outer frame member 49556 deflects from the expanded configuration to the narrow configuration. Since the connector 29566 both pulls the upper or proximal portions of the medial portion 49578 and the lateral portion 49580 through the proximal guide 49900 and pulls the lower or distal portions of the medial portion 49578 and the lateral portion 49580 through the distal guide 49901 by the same amount, the portion of the outer frame member 49556 that engages the native valve leaflet does not move upward or proximally or downwardly or distally.

In the example illustrated by FIG. 44, the connector 29566 is moved up or proximally, opposite to the direction indicated by arrow 49700 to move the outer frame member 49556 from the contracted or narrow configuration toward the expanded configuration. The connector pushes the distal portions of the medial portion 49578 and the lateral portion 49580 through the choke point created by the distal guide 49901, opposite the direction as indicated by arrow 49701. As a result, the distal connection portion 49671 pushes the proximal portions of the medial portion 49578 and the lateral portion 49580 through the choke point created by the proximal guide 49900, opposite the direction as indicated by arrow 49703. In some implementations, the outer frame member 49556 deflects opposite to the directions to that indicated by arrows 49704 from the narrow configuration toward the expanded configuration.

In some implementations, such as in the example illustrated by FIG. 44, the outer frame member 49556 only laterally engages the native valve leaflet, such the outer frame member does not pull the native leaflet further into the device 49500 or urge the native leaflet out of the device when the outer frame member is expanded or contracted.

In some implementations, the two outer frame members 49556 move apart or separate as the outer frame members are moved from the expanded configuration to the narrow configuration. In some implementations, the medial portion 49578 and the lateral portion 49580 of the outer frame members 49556 are shaped and/or configured such that the two outer frame members 49556 stay in contact with one another or stay close to one another as the outer frame members are moved from the expanded configuration to the narrow configuration.

In some implementations, the medial portion 49578 and a lateral portion 49580 that are distal to the proximal guide 49900 and that are distal to the distal guide 49901 are close together (i.e., due to passing through the guides). In some implementations, the medial portion 49578 and a lateral portion 49580 that are on the other side of the guides 49900 move toward and away from one another to change the width of the outer frame member 49556.

In some implementations, each of the devices 29500, 39500, 49500 have a proximal or attachment portion 29505, paddle frames 29524, inner paddles 29525, outer paddles 29526, and a distal portion 29507. The proximal portion 29505, the distal portion 29507, the paddle frames 29524, and the paddles 29525, 29526 can be configured in a variety of ways, such as any configuration disclosed herein.

The example devices 29500, 39500, 49500 illustrated in FIGS. 42-44 can be the same as or similar to the device 8200 of FIG. 26. For example, the paddle frames 29524 can be symmetric along a longitudinal axis YY (FIG. 26). However, in some implementations, the paddle frames 29524 are not symmetric about the longitudinal axis YY. The paddle frames 29524 include the outer frame member or members 29556 (e.g., wire frame portion, metal frame portion, etc.) and an inner frame member or members 29560 (e.g., wire frame portion, metal frame portion, etc.).

In some implementations, the outer frame members 29556, 39556, 49556 are flexibly attached to the connector 29566. In some implementations, the outer frame members 29556 form a curved shape. For example, in the illustrated example, the shape of the outer frame members 29556, 39556, 49556 can resemble an oval shape, a double “D” shape, etc. The outer frame members 29556, 39556, 49556 can be otherwise shaped.

In some implementations, the inner frame members 29560 extend from the proximal portion 29505 toward the distal portion 29507. In some implementations, the inner frame members 29560 then extend inward to form retaining portions 29572 that are attached to an actuation cap 29514, in the same or similar manner as described in relation to any of the example devices described herein. The retaining portions 29572 and the actuation cap 29514 can be configured to attach in any suitable manner.

In some implementations, the inner frame members 29560 are rigid frame portions, while the outer frame members 29556 are flexible frame portions. In some implementations, the devices 29500, 39500, 49500 are configured to move the outer frame members 29556 from an expanded position to a narrowed position by pulling portions of the connector 29566 into the actuation cap 29514. In some implementations, the devices 29500, 39500, 49500 are configured to move the inner frame members 29560 to open and close the paddles 29525, 29526 in accordance with some implementations disclosed herein.

In some implementations, as illustrated in the examples of FIGS. 42-44, the devices 29500, 39500, 49500 each include the pair of adjustable width frame members, one arranged anteriorly and the other arranged posteriorly. In particular, the devices 29500, 39500, 49500 include an anterior adjustable width frame member and a posterior adjustable width frame member, both of which connect to a connector 29566. In the devices 29500, 39500, 49500, the connector 29566 can optionally be arranged to extend anteriorly and posteriorly as opposed to medially and laterally as with the device 8200 of FIGS. 26-30. In some implementations, the connector 29566 of the devices 29500, 39500, 49500 can optionally be arranged to extend medially and laterally in the same manner as the device 8200 of FIGS. 26-30.

The anterior adjustable width frame member and the posterior adjustable width frame member can be formed in any suitable manner. For example, anterior adjustable width frame member and the posterior adjustable width frame member can include a shape-memory alloy, such as Nitinol, to provide shape-setting capability. Each of the anterior adjustable width frame member and the posterior adjustable width frame member can be shape-set into the as-assembled condition or assembled in a flat condition or profile. In the illustrated example, the anterior adjustable width frame member and the posterior adjustable width frame member are identical. In some implementations, however, the anterior adjustable width frame member and the posterior adjustable width frame member can be configured differently.

In some implementations, the connector 29566 includes a proximal portion 29586 adjacent the distal portions of the adjustable width frame member 29556, 39556, 49556. In some implementations, the proximal portion 29586 includes an attachment portion 29590 for connecting to the adjustable width frame member 29556, 39556, 49556. In some implementations, the attachment portion 29590 can be configured in a variety of ways. In some implementations, the attachment portion 29590 includes a loop or hole for connection to the adjustable width frame member 29556, 39556, 49556.

In some implementations, the attachment points between the adjustable width frame member 29556, 39556, 49556 and the connector 29566 can act as hinges allowing for free rotation or pivoting between the adjustable width frame member 29556, 39556, 49556 and the connector 29566 at the attachment point(s) resulting in reduced stress and tension on the delivery system and device.

In some implementations, the devices 29500, 39500, 49500 are configured to move the adjustable width frame members 29556, 39556, 49556 from an expanded position to a narrowed position by pulling portions of the connector into the actuation cap in the same manner as the connector 29566 and actuation cap 29514 of FIG. 31. In some implementations, the devices are also configured to move the inner frame members 29560 to open and close the paddles (e.g., outer paddles 320 and inner paddles 322 of FIG. 23) in accordance with some implementations disclosed herein.

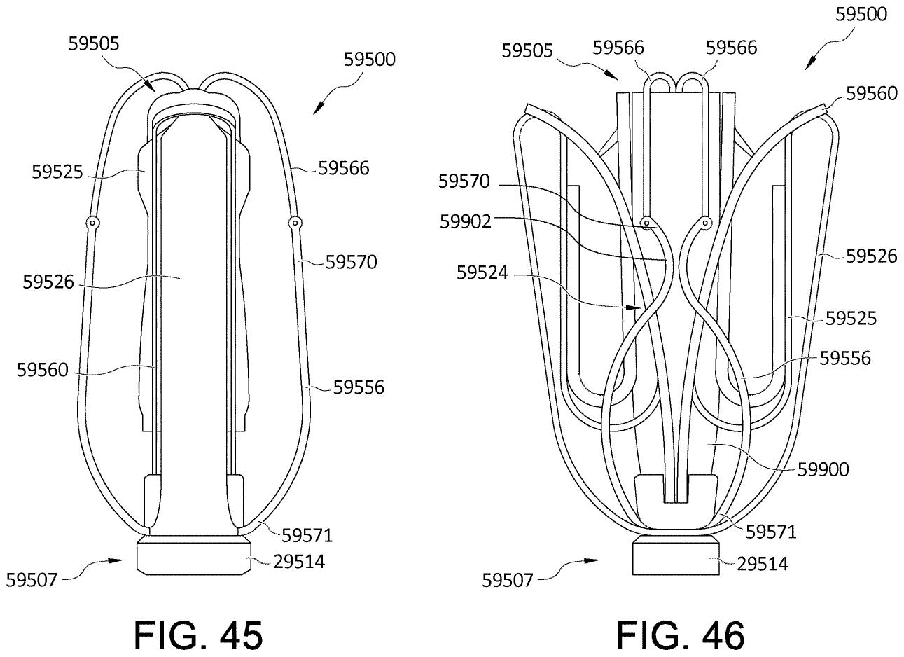

FIGS. 45-48 illustrate an example of one of the many systems (e.g., a treatment system, a repair system, a valve repair system, a valve treatment system, etc.) useable for treating and/or repairing a native valve of a patient that the concepts of the present application can be applied to.

In some implementations, the device 59500 includes an expandable frame member 59556 that is connected to or coupled to a distal portion 59507 of the device 59500 and a connector 59566 that extends into and out of a proximal portion 59505 of the device 59500.

Referring to FIGS. 45 and 46, in some implementations, the expandable frame member 59556 contracts or narrows when a portion of the connector 59566 of the device 59500 is pulled into the proximal portion 59505 of the device 59500.

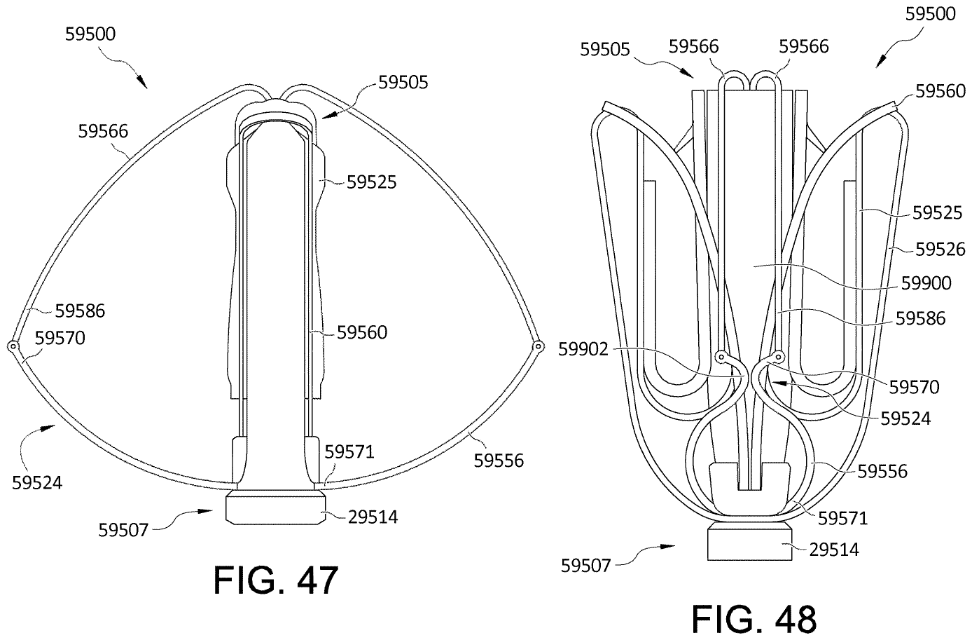

Referring to FIGS. 47 and 48, in some implementations, the expandable frame member 59556 expands when a portion of the connector 59566 of the device 59500 is pushed out of the proximal portion 59505 of the device 59500.

In some implementations, the expandable frame member 59556 can be connected to or coupled to the distal portion 59507 of the device in a variety of different ways. In some implementations, the expandable frame member 59556 can be attached to a cap 29514 of the device 59500 or a body or base 59900 of the device that the cap moves relative to. The connector 59566 can extend into and out of a proximal portion 59505 of the device 59500 in a variety of different ways. In some implementations, the connector 59566 can extend into a collar (see the collar 211) and/or the body or base 59900 of the device.

In some implementations, the portion 59902 of the expandable frame member. 59556 that engages the native valve leaflet moves generally downward or distally as the expandable frame member 59556 deflects from the narrow configuration (FIGS. 45 and 46) to the expanded or wide configuration (FIGS. 47 and 48). As such, the expandable frame member 59556 can pull the native valve leaflet further into the device as the outer frame member 59556 moves from the narrow configuration to the wide configuration.

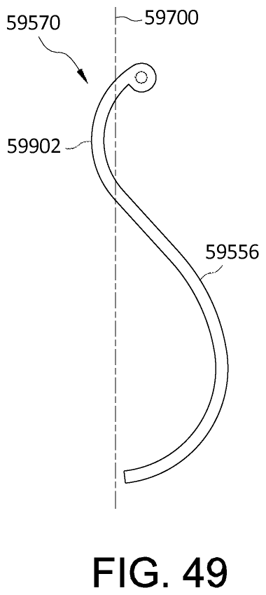

Referring to FIGS. 46, 48 and 49, in some implementations, the expandable frame members 59556 are shaped and/or configured such that the portions 59902 of the two expandable frame members 59556 stay in contact with one another or stay close to one another as the outer frame members are moved from the expanded configuration to the narrow configuration. For example, each of the expandable frame members 59556 can be shape set such that the portion 59902 of the expandable frame member that engages the other expandable frame member has a normal position that is past a centerline 59700 of the device as illustrated by FIG. 49. As a result, the two expandable members 59556 can stay in contact in the expanded condition, the narrow condition and the positions between the expanded condition and the narrow condition.

In some implementations, the two expandable frame members 59556 move apart or separate as the frame members are moved from the expanded configuration to the narrow configuration.

In some implementations, a system (e.g., a treatment system, a repair system, a valve repair system, a valve treatment system, etc.) includes a treatment and/or repair device 59500 (e.g., a treatment device, a repair device, a valve repair device, a valve treatment device, a prosthetic device, an implantable device, an implant, etc.) having a proximal or attachment portion 59505, one or more paddle frames 59524, one or more inner paddles 59525, one or more outer paddles 59526, and a distal portion 59507. The proximal portion 59505, the distal portion 59507, the paddle frames 59524, and the paddles 59525, 59526 can be configured in a variety of ways, such as any configuration disclosed herein.

The example device 59500 illustrated in FIGS. 45-48 can be the same as or similar to the device 8200 of FIG. 26. For example, the paddle frames can be symmetric along a longitudinal axis YY (FIG. 26). However, in some implementations, the paddle frames are not symmetric about the longitudinal axis YY. The paddle frames can include the expandable frame member or members 59556 (e.g., wire frame portion, metal frame portion, etc.) and an inner frame member or members 59560 (e.g., wire frame portion, metal frame portion, etc.). In some implementations, the expandable frame members 59556 are flexibly attached to the connector 59566. The expandable frame members 59556 optionally form a curved shape.

In some implementations, the inner frame members 59560 extend from the proximal portion 59505 toward the distal portion 59507. In some implementations, the inner frame members 59560 can include retaining portions that are attached to the actuation cap 29514, in the same or similar manner as described in relation to any of the example devices described herein. The retaining portions and the actuation cap 29514 can be configured to attach in any suitable manner. In some implementations, the inner frame members 59560 are rigid frame portions, while the expandable frame members 59556 are flexible frame portions.

In some implementations, the device 59500 is configured to move the outer frame members 59556 from an expanded position (FIGS. 47 and 48) to a narrowed position (FIGS. 45 and 46) by pulling portions of the connector 59566 into the proximal portion 59505 of the device 59500. In some implementations, the device 59500 is configured to move the inner frame members 59560 to open and close the paddles 59525, 59526 in accordance with some implementations disclosed herein.

In some implementations, as illustrated in the example device 59500 in FIGS. 45-48, the device 59500 includes the pair of adjustable width frame members 59556, one arranged anteriorly and the other arranged posteriorly. In particular, the device 59500 includes an anterior adjustable width frame member and a posterior adjustable width frame member, both of which connect to the connector 59566. The connector 59566 can be arranged to extend anteriorly and posteriorly or medially and laterally.

The anterior adjustable width frame member and the posterior adjustable width frame member can be formed in any suitable manner. For example, the anterior adjustable width frame member and the posterior adjustable width frame member can include a shape-memory alloy, such as Nitinol, to provide shape-setting capability. Each of the anterior adjustable width frame member and the posterior adjustable width frame member can be shape-set into the as-assembled condition or assembled in a flat condition or profile. In the illustrated example, the anterior adjustable width frame member and the posterior adjustable width frame member are identical. Thus, the description below of the anterior adjustable width frame member applies equally to the posterior adjustable width frame member. In some implementations, however, the anterior adjustable width frame member and the posterior adjustable width frame member can be configured differently.

The adjustable width frame member 59556 (e.g., wire frame portion, metal frame portion, etc.) can be configured in a variety of ways, including different shapes and sizes. Referring to FIGS. 45-49, in the illustrated example, each adjustable width frame member 59556 is formed with an open proximal portion 59570, a closed distal portion 59571 opposite the proximal portion 59570. In some implementations, however, the adjustable width frame member 59556 can be a closed loop.

In some implementations, the proximal portion 59570 is connected to a distal portion 59586 of the connector 59566 at, or adjacent, the midplane of the device 59500. In some implementations, the proximal portion 59570 of the adjustable width frame member 59556 can connect to the distal portion 59586 of the connector 59566 in a variety of ways. In some implementations, the proximal portion 59586 accommodates connection elements, such as, for example, sutures, fasteners, adhesives, etc. for attaching the proximal portion 59570 to the connector 59566. In some implementations, the attachment points between the adjustable width frame member 59556 and the connector 59566 can act as hinges optionally allowing for free rotation or pivoting between the adjustable width frame member 59556 and connector 59566 at the attachment point resulting in reduced stress and tension on the delivery system and device.

In some implementations, the device is configured to move the adjustable width frame members from an expanded position to a narrowed position by pulling portions of the connector into the proximal portion 59505 of the device 59500. In some implementations, the connector 59566 can be pulled into a collar (see the collar 211) and/or the body or base 59900 of the device 59500 to move the adjustable width frame members from an expanded position to a narrowed position. In some implementations, the device is also configured to move the inner frame members 59560 to open and close the inner paddles 59525 and the outer paddles 59526 in accordance with some implementations disclosed herein.

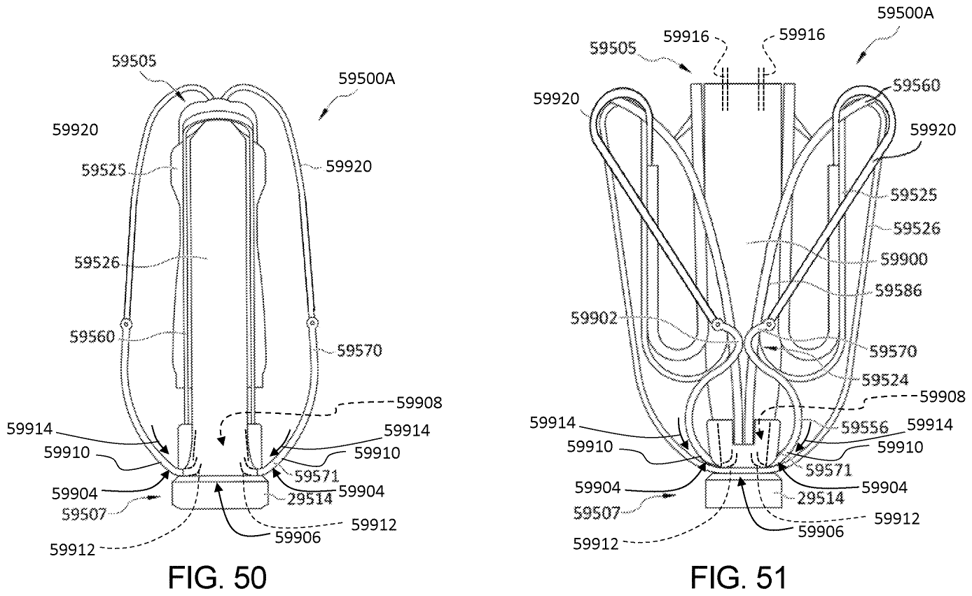

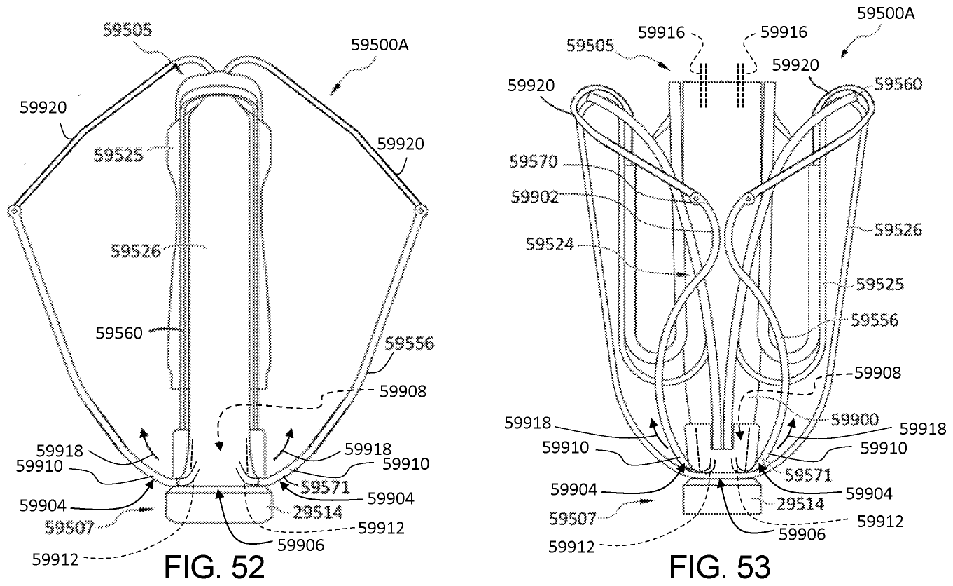

Referring now to FIGS. 50-53, an implementation of a device 59500A similar to device 59500 shown in FIGS. 45-48 is illustrated. Device 59500A uses portions of the adjustable paddle frames to move the paddle frames from the narrowed (FIGS. 50 and 51) to the expanded positions (FIGS. 52 and 53).

In an example, portions of the paddle frames can extend and retract from the distal end of a central body of the device 59500A. In some implementations, the central body can comprise one or more of the base, spacer, center support member, receiver, or bushing. The paddle frames extending and retracting from the distal end of the central body can affect the change in position from the narrowed position to the expanded position. By using the distal end of the central body of the device (e.g., base, spacer, center support member, receiver, or bushing) and extension and retraction of portions of the paddle frames therefrom, this arrangement provides additional space or clearance for native leaflet tissue to be captured in the region between the expanded paddles/frame portions.

Referring to FIGS. 50 and 51, front and side elevation views of an implementation of device 59500A with the paddle frame portions in the collapsed or narrowed position are illustrated. The arrangement in device 59500A extends and retracts portion(s) of the adjustable paddle frames from the distal end of the central body of the device (e.g., base, spacer, center support member, receiver, or bushing) proximate the cap. In comparison, the arrangement of device 59500 in FIGS. 45-48 extends and retracts the frame portions from the proximal end of the central body. As such, the frame portions of the device 59500 act as expandable spacer portions or expandable spacer frames (positioned between the leaflets when implanted) and the frame portions 59500A act as expandable paddle frames (on the outside of the leaflets when implanted).

In the implementation of device 59500A, frame portions 59571 are retracted into and extended from distal end portion 59906 of the central body of the device (e.g., base, spacer, center support member, receiver, or bushing). FIGS. 50 and 51 illustrate the configuration where frame portions 59571 include movable frame portions 59904 that have been retracted into the distal end portion 59906 to provide a narrowed position of the adjustable paddle frames. More specifically, movable frame portions 59904 can include an outer frame portion 59910 and an inner frame portion 59912. Outer frame portion 59910 can extend from distal end portion 59906 of the central body and inner frame portion 59912 can reside inside distal end portion 59906. In the implementation shown, outer frame portion 59910 and inner frame portion 59912 have curved or arcuate shapes or portions permitting the frame portions to bend or flex around distal end portion 59906 and into inner space 59908. In other examples, different shapes can be used to accomplish the same or similar configuration and functionality of the movable frame portions 59904.

In some implementations, the central body, (e.g., base, spacer, center support member, receiver, or bushing's body) includes an inner space 59908 from which movable frame portions 59904 can be extended from and retracted into. In some implementations, inner space 59908 can be one or more open spaces or regions in spatial communication with the distal end portion 59906 such that components can extend from inner space 59908 and out of distal end portion 59906 and vice-versa. Inner space can extend from the proximal portion to the distal end portion 59906 of the central body. In other examples, inner space 59908 extends a portion of the length of the central body.

In some implementations, one or more actuation elements 59916 (lines, sutures, wires, chords, rods, tubes, etc.) are provided and connect to movable frame portions 59904. Actuation elements 59916 can translate or move to thereby cause movable frame portions 59904 to extend and/or retract from distal end portion 59906 of the central body (e.g., base, spacer, center support member, receiver, or bushing). As such, actuation elements 59916 can extend through the central body and connect to movable frame portions 59904. In some implementations, actuation elements 59916 can be extensions of movable frame portions 59904. In some implementations, actuation elements can be separate elements connected to the movable frame portions 59904.

As previously described, the paddle frame portions can be made of flexible materials such as, for example, Nitinol, which allow the paddle frame portions to change shape, bend, flex, be shape-set, have shape memory, etc. Actuation elements 59916 can also be made of the same or similar materials if being a separate component from movable frame portions 59904. Actuation elements 59916 can be actuated or moved by any one or more mechanisms or components including, for example, paddle actuation control 1626 and/or paddle width control 1628, which can be located in a control handle 1616 of a catheter-based implantable device system.

Arrows 59914 in FIGS. 50 and 51 indicate the direction of movement for movable frame portions 59904 when retracting movable frame portions 59904 into distal end portion 59906 to position the paddle frames in the narrowed position. In the implementation shown, movement of movable frame portions 59904 shortens the length of outer frame portion 59910 thereby drawing inward or collapsing paddle frame portion 59920 to provide the paddle frames with the collapsed or narrowed position illustrated.

Referring now to FIGS. 52 and 53, front and side elevation views of an implementation of device 59500A with the paddle frame portions in the expanded position are illustrated. In this arrangement, paddle frame portions 59571 have been extended in the direction of arrows 59918 from the distal end portion 59906 to provide an expanded position of the paddle frames. Extending frame portions 59571 in the direction of arrows 59918 allows movable frame portions 59904 to expand outward to their shape-set configuration. This is because outer frame portion 59910 gets longer as it is extended from distal end portion 59906, which allows paddle frame portion 59920 to move or swing outward to achieve the expanded configuration shown. Hence, by lengthening and shortening outer frame portion 59910 of movable frame portions 59904, paddle frame portion 59920 can moved from a narrowed (or collapsed) position to an extended position.

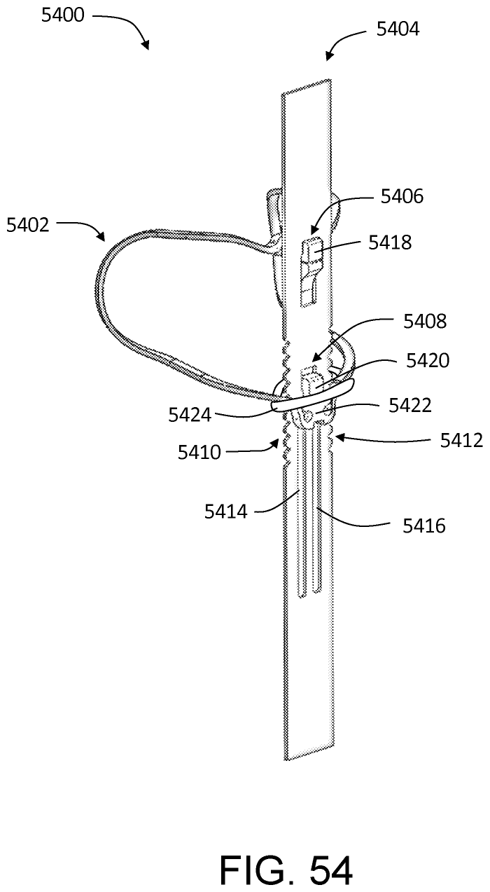

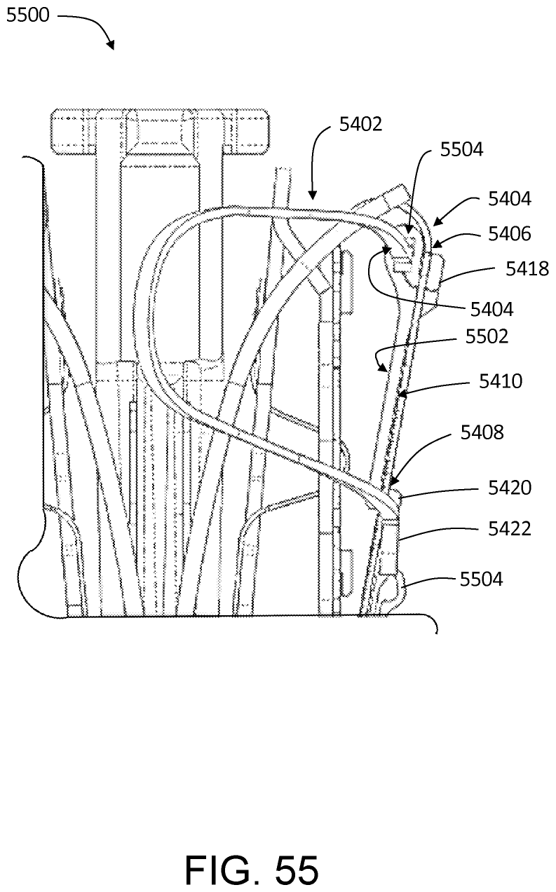

Referring now to FIGS. 54 and 55, an implementation of a system 5400 and device 5500 for expanding paddle frames is illustrated. In some implementations, device 5500 may be similar to devices described elsewhere in this application (e.g., device 29505). That is, device 5500 may include like components such as a central body, one or more anchors, one or more paddles, etc.

FIG. 54 shows a partial perspective of the system 5400 in isolation with certain parts of the implantable device not shown. FIG. 55 shows a partial side elevational view of a device 5500 having system 5400. System 5400 is an implementation or arrangement for expanding and/or narrowing paddle frame(s) 5402 of which there are normally one or more per side (e.g., front and back or left and right) of the implantable device (as described throughout this disclosure). Guides, such as, for example, guides 49900 and/or 49901 (e.g., shown in FIG. 44) (other guides disclosed herein) can either be included or omitted in this implementation. In the implementation shown in FIGS. 54 and 55, a guide 5424 is shown, which can be according to any of the examples disclosed herein including, for example, a suture, wire (e.g., nitinol), or other guide arrangement disclosed herein.

System 5400 includes paddle frame(s) 5402 and paddle portion 5404. Paddle portion 5404 can be any portion of the paddles including inner and/or outer portions. In some implementations, paddle portion 5404 is part of the outer paddle structure (e.g., see outer paddle 29526 in FIG. 44 and other examples disclosed herein). As will be described in more detail, paddle frame(s) 5402 can be connected to paddle portion 5404 and arranged to be expanded and/or narrowed, as previously described throughout this disclosure.

In some implementations, paddle portion 5404 includes openings 5406 and 5408 and slot apertures 5414 and 5416. Openings 5406 and 5408 are sized to accept clip portions 5418 and 5420 that mount and fixate paddle frame attachment body 5502 to paddle portion 5404. The attachment between the clip portions 5418 and 5420 and the openings 5406 and 5408 can be a snap-fit, interference fit, clip-on or similar type arrangement. In some implementations, clip portions 5418 and 5420 can be cantilevered lug portions having projecting beams to form a hook-type arrangement for clipping into openings 5406 and 5408. In some implementations, attachment can be via fasteners, welds, glues and/or adhesives.

The paddle frames 5402 can be connected to the paddles at a first location through attachment portion 5504 of the mount and fixate paddle frame attachment body 5502. Attachment portion 5504 can include, in one example, an opening or aperture for receiving and fixating a portion of paddle frame(s) 5402A, as shown in FIG. 55. Paddle frame(s) 5402 are secured via a second location through paddle frame connection portion 5422, which includes pin-like protrusions (or projections) for riding within slot apertures 5414 and 5416. While two slot apertures are illustrated, one or more can be used. A connector 5504 (see also, connector 29566 (FIG. 42) and other examples of connectors disclosed herein) can be connected to paddle frame connection portion 5422 via a hook, loop, suture, wire or other arrangement disclosed herein.

In some implementations, paddle portion 5404 includes first and second side portions having fixating portions 5410 and 5412, respectively. In some implementations, fixating portions 5410 and 5412 can include undulating surfaces such as, for example, a tooth or saw tooth or other varying pattern and can position guide 5424 along the length thereof. So arranged, fixating portions 5410 and 5412 provide a plurality of positions along the length of paddle portion 5404. In some implementations, the fixating portions 5410 and 5412 includes one or more optional recesses for retaining or fixating guide 5424 in position to create one or more constriction regions for narrowing the paddle frame(s) 5402, as previously discussed herein. Slot apertures 5414 and 5416 define a linear path of movement along the length for paddle frame connection portion 5422 as it moves along paddle portion 5404 to expand or narrow the paddle frame(s) 5402.

In operation, the connector (e.g., 29566 in FIG. 42) pulls on paddle frame connection portion 5422 resulting in portions of the paddle frame(s) 5402 moving or passing through the choke or constriction region created by guide 5424 and its fixation to paddle portion 5404. The movement of paddle frame connection portion 5422 is guided by slot apertures 5414 and 5416, which can define the upper and lower distance/position limits of the slotted movement.

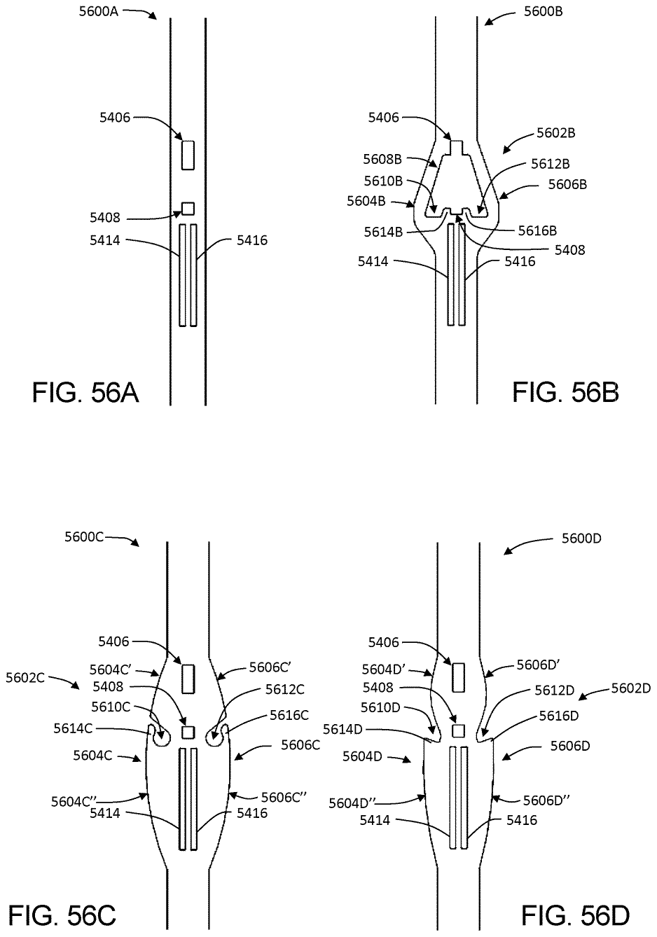

FIGS. 56A, 56B, 56C and 56D illustrate further implementations of paddle portions. FIG. 56A illustrates a system 5600A similar to system 5400 of FIG. 54, except without fixating portions 5410 and 5412. Slot apertures 5414 and 5416 are included for guided movement of the paddle frames connection portion 5422 as previously described. In this implementation, guides similar to guide 29900 (FIG. 42) can be used. Other guide arrangements can also be used as disclosed herein.

FIG. 56B illustrates a system 5600B wherein the paddle portion includes a distended portion 5602B. Distended portion 5602B includes first side 5604B and second side 5606B, which progressively widen to create the distension of portion 5602B. Within distended portion 5602B is a guide portion 5608B. Guide portion 5608B is shown generally triangular or trapezoidal, though other shapes can be used. Guide portion 5608B includes portions 5610B and 5612B, which can be cutouts, recesses, or inset apertures/openings and provide regions or areas of constriction for paddle frame members similar to the previously described guides (e.g., see FIGS. 38-44) that constrict, restrict, and/or choke movement of the paddle frame members during expansion and/or narrowing.

In the implementation shown, left and right side guides 5610B and 5612B, respectively, each receive a portion of a paddle frame member therethrough and restrict its lateral movement while allowing its longitudinal movement to effect expansion and narrowing of the paddle frame. Spaced apart projecting members or tabs 5614B and 5616B project into the inner space of guide portion 5608B to provide end wall portions of guides 5610B and 5612B and also to provide end wall portions for attachment or mounting opening 5408. Guide portions 5610B and 5612B are shown having linear or rectilinear inner bearing wall surfaces. In some implementations, these wall surfaces can be arched or curved (e.g., similar to that shown in FIGS. 56C and 56D). Attachment or mounting opening 5406 is formed in the perimeter wall of guide portion 5608B as shown. In some implementations, openings 5406 and 5408 can be formed separately as discrete openings (e.g., as shown in FIG. 56A). In operation, paddle frame members are positioned within guides 5610B and 5612B, which constrict the lateral movement of the paddle frame portions while allowing their longitudinal movement to effect expansion and narrowing of the paddle frames.

FIG. 56C illustrates a system 5600C wherein the paddle portion includes a distended portion 5602C. Distended portion 5602C includes two distended portions. In some implementations, more or less distended portions may be included. A first distended portion is provided by side portions 5604C′ and 5606C′ and a second portion includes side portions 5604C″ and 5606C″. Within or between these distended side portions, guide portions 5610C and 5612C are formed. Guide portions 5610C and 5612C, which can be cutouts, recesses, or inset apertures/openings and provide regions or areas of constriction and/or choke areas for paddle frame members similar to the previously described guides (e.g., see FIGS. 38-44) that constrict, restrict, and/or choke the paddle frame members movement during expansion and/or narrowing.

In the implementation shown, left and right side guides 5610C and 5612C, respectively, each receive a portion of a paddle frame member therethrough and restrict its lateral movement while allowing its longitudinal movement to effect expansion and narrowing of the paddle frame. Spaced apart projecting members or tabs 5614C and 5616C project from sides 5604C and 5606C to provide wall portions of guides 5610C and 5612C. Guide portions 5610C and 5612C are shown having arched or curved inner bearing wall surfaces that almost for a complete circuit, except for a small entry channel for inserting the paddle frame members. In some implementations, these wall surfaces can be linear or rectilinear similar to that shown in FIG. 56B. In operation, paddle frame members are positioned within guides 5610C and 5612C, which constrict the lateral movement of the paddle frame portions while allowing their longitudinal movement to effect expansion and narrowing of the paddle frames.

FIG. 56D illustrates a system 5600D wherein the paddle portion includes a distended portion 5602D. Distended portion 5602D includes two distended portions. A first distended portion is provided by side portions 5604D′ and 5606D′ and a second distended portion is provided by side portions 5604D″ and 5606D″. Within or between these distended side portions, guide portions 5610D and 5612D are formed. Guide portions 5610D and 5612D, which can be cutouts, recesses, or inset apertures/openings and provide regions or areas of constriction and/or choke points for paddle frame members similar to the previously described guides (e.g., see FIGS. 38-44) that constrict, restrict, and/or choke the paddle frame members movement during expansion and/or narrowing. Although shown with two distended portions additional distended portions may be included without departing from the scope of this disclosure.

In the implementation shown, left and right-side guides 5610D and 5612D, respectively, each receive a portion of a paddle frame member therethrough and restrict its lateral movement while allowing its longitudinal movement to effect expansion and narrowing of the paddle frame. Spaced apart projecting members or tabs 5614D and 5616D project from sides 5604D″ and 5606D″ to provide wall portions of guides 5610C and 5612C. Guide portions 5610C and 5612C are shown having arched or curved inner bearing wall surfaces. In some implementations, these wall surfaces can be linear or rectilinear similar to that shown in FIG. 56B. In operation, paddle frame members are positioned within guides 5610D and 5612D, which constrict the lateral movement of the paddle frame portions while allowing their longitudinal movement to effect expansion and narrowing of the paddle frames.

In these implementations (e.g., FIGS. 56A, 56B, 56C, and 56D), additional guide elements can optionally be included such as, for example, guide 5424, which can be according to any of the examples disclosed herein including, for example, a suture, wire (e.g., nitinol), or other guide arrangement disclosed herein. Hence, the implantable devices disclosed herein can include paddles having one or more guide elements in the structure of the paddle themselves that can facilitate the narrowing and expansion of the associated paddle frames.

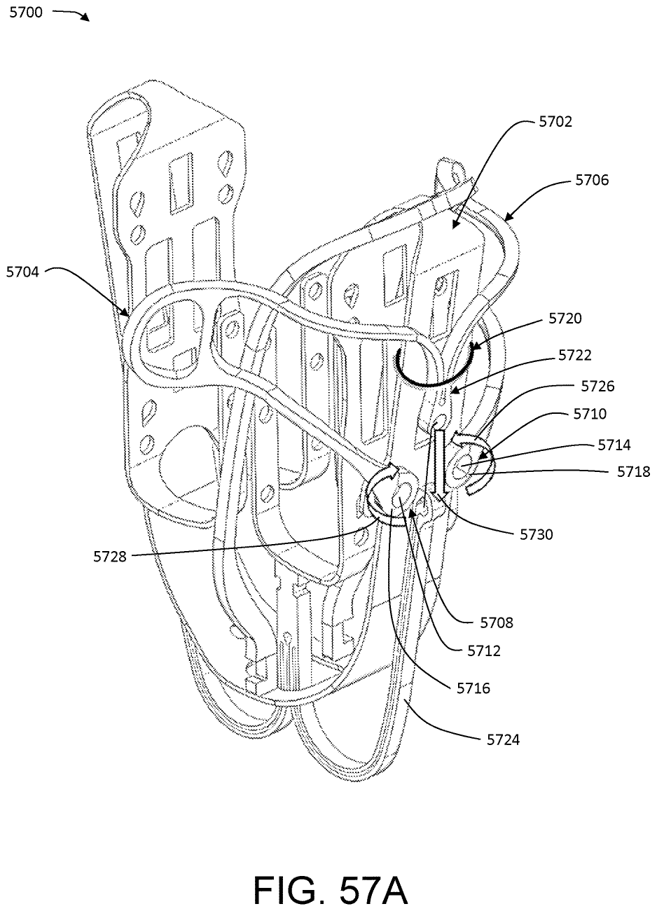

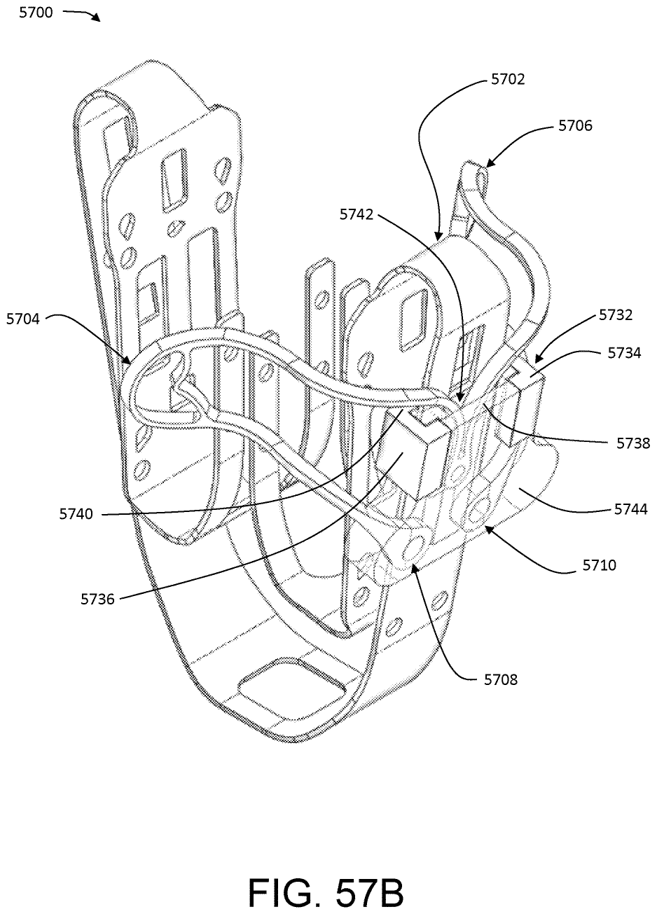

Referring now to FIGS. 57A-65, some implementations of implantable devices for heart valve repair are shown. As explained throughout this disclosure, the implementations include adjustable width paddle frames that assist in native leaflet capture. The paddle frames can be adjustable from a narrowed or collapsed arrangement suitable for delivery of the implantable device (e.g. to traverse more easily through chordae tendinea and/or for passing through a catheter) to an expanded and/or capture-ready arrangement suitable for capture of native heart valve leaflets and/or to engage a wider area of the native heart valve leaflets. The implementations of FIGS. 57A-65 show further implementations of such devices.

Referring now to FIGS. 57A-57B, some implementations of an implantable device 5700 having a pivot or hinged joint or connection for the adjustable width paddle frames 5704 and 5706 are shown. In FIGS. 57A-57B, the paddles and paddle frames have been shown in isolation from other components of the device (e.g., central bushing, cap, retaining element, actuation elements, etc.), which can be found throughout the disclosure including in, for example, FIGS. 22-23, 26-34, and 38-56, and the associated text. Referring now to FIG. 57A, implantable device 5700 includes a paddle 5702 and first and second adjustable width paddle frames 5704 and 5706. A similar set of paddle frames are present on the second paddle and not shown but with the understanding that the present disclosure is equally applicable.

The device 5700 can also include hinge or pivot joint portions 5708 and 5710 that allow movement of adjustable width paddle frames 5704 and 5706. In some implementations, the movement is a pivoting or hinge movement about a region or area. The pivot portions 5708 and 5710 can include, for example, pivot pin portions 5712 and 5714 located on paddle 5702 and pivot ring portions 5716 and 5718 located near the base region of adjustable width paddle frames 5704 and 5706. In some implementations, the arrangement can be reversed with pivot pin portions 5712 and 5714 located on paddle 5702 and pivot pin portions 5714 and 5716 located on adjustable width paddle frames 5704 and 5706. As will be described further below, hinge or pivot portions 5708 and 5710 allow adjustable width paddle frames 5704 and 5706 to move in a pivoting or hinging motion as they change from a narrowed to an expanded position.

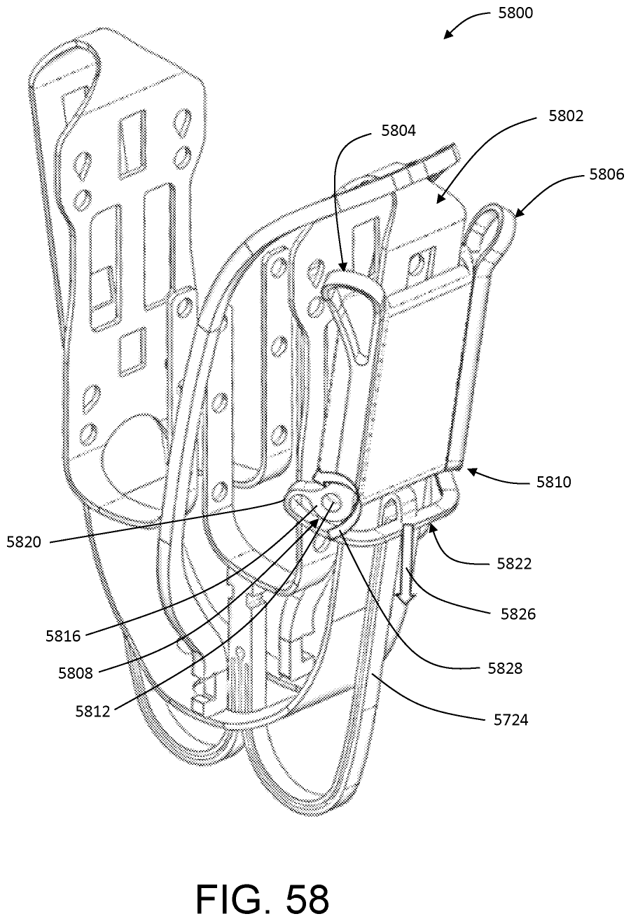

In some implementations, hinge or pivot portions 5708 and 5710 are positioned a distance approximately halfway along the longitudinal length of the outer paddle portion of paddle 5702 as shown in FIG. 57A (and 57B). However, in some implementations, hinge or pivot portions 5708 and 5710 can be positioned along distances that are a quarter, third, half, two-thirds, three-quarters, etc., along the longitudinal length of the paddle structure, (e.g., outer paddle structure). In the implementation shown, hinge or pivot portions 5708 and 5710 are arranged co-planar, substantially co-planar, and/or in a plane parallel offset with the plane of the outer paddle structure of paddle 5702. In some implementations, such as that shown in FIG. 58, hinge or pivot portions 5708 and 5710 can be angularly offset from the plane of the outer paddle structure of paddle 5702.

In some implementations, the device 5700 can include one or more guides 5720. One or more guides 5720 can be according to any of the previously described implementations. The one or more guides 5720 can constrict, restrict and/or choke movement of portions of adjustable width paddle frames 5704 and 5706 as the adjustable width paddle frames are moved from their narrowed to expanded (or vice-versa) positions.

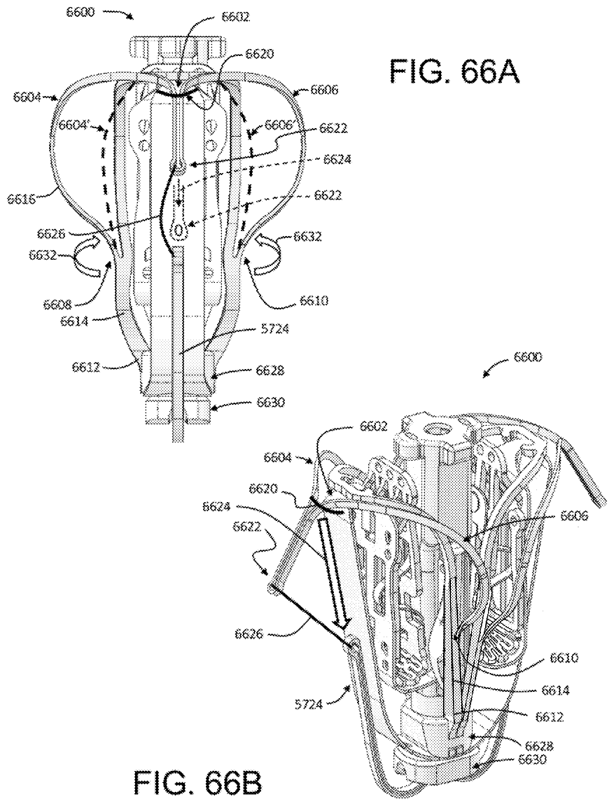

Adjustable width paddle frames 5704 and 5706 can include a distal connection portion 5722. Distal connection portion 5722 functions in the same manner as other distal connection portions described herein (e.g., see reference number 39670 (FIG. 43), reference number 49671 (FIG. 44), etc., and associated text) to effect movement of adjustable width paddle frames 5704 and 5706. Distal connection portion 5722 can include, for example, one or more portions for connecting to an actuation connector 5724 that can move longitudinally. In some implementations, connection portion 5722 can include an aperture, slot, flare, recess, cutout, loop, hook, etc., or any other implementation described herein. Distal connection portion 5722 can be connected to actuation connector 5724 via any of the described examples herein including, for example, wire, suture, sleeve, etc. Distal connection portion 5722 can also be shape set to be biased inwardly or outwardly from implantable device 5700. (e.g., see FIG. 66B showing distal connection portion 6622 shape set with an outward bias.) This can provide a pre-load on, for example, actuation connectors 5724 and paddle frames 6404 and 6406. In some implementations, a bias or pre-load is not be used.

In operation, adjustable width paddle frames 5704 and 5706 can be shape set to the expanded configuration shown in FIG. 57A. Downward movement as represented by arrow 5730 of actuation connector 5724 causes downward movement of connection portion 5722. This draws portions of adjustable width paddle frames 5704 and 5706 through one or more guides 5720 and causes adjustable width paddle frames 5704 and 5706 to pivot or rotate about hinge or pivot portions 5708 and 5710 as indicated by arrows 5726 and 5728. This pivoting or rotational movement causes adjustable width paddle frames 5704 and 5706 to change from their expanded to their narrowed or collapsed position where they are drawn closer to the body of implantable device 5700. This arrangement can be referred to as passively expanding paddle frames, since the paddles can be shape set in the expanded state and will return to the expanded state in the absence of the pulling force 5730.

Referring now to FIG. 57B, device 5700 is shown with guide 5732. Guide 5732 can be in the form of a conduit, channel, or passageway 5742, which can be partially or fully enclosed by a guide body. The guide 5732 body can include a plurality of walls 5734, 5736, 5738 and 5740 that partially and/or fully include distal connection portion 5722. The guide 5732 body can be any shape including rectangular, polygonal, circular and/or non-circular. Additional wall portions including wall portions 5744 can be included which cover partially or fully regions having or near hinge or pivot portions 5708 and 5710. Guide 5732 can be attached to any location on paddle 5702 including, for example, the outer paddle structure shown in FIG. 57A. Similar to the previously described guides herein, guide 5732 acts to guide portions of adjustable width paddle frames 5704 and 5706 as the paddle frames are changed from their narrowed to expanded positions. In this implementation, guide 5732 uses its passageway 5742 to restrict and guide the movement of adjustable width paddle frames 5704 and 5706.

Referring now to FIG. 58, an implantable device 5800 having a pivot or hinged connection for the adjustable width paddle frames 5804 and 5806 is shown. Similar to FIGS. 57A-57B, the paddles and paddle frames have been shown in isolation from other components of the device (e.g., central bushing, cap, retaining element, actuation elements, etc.), which can be found throughout the disclosure including in, for example, FIGS. 22-23, 26-34, and 38-56, and the associated text. Implantable device 5800 includes a paddle 5802 and first and second adjustable width paddle frames 5804 and 5806. A similar set of paddle frames are present on the second paddle and not shown but with the understanding that the present disclosure is equally applicable.

Implantable device 5800 includes hinge or pivot joint portions 5808 and 5810 that allow movement of adjustable width paddle frames 5804 and 5806. In this implementation, the movement is a pivoting or hinge movement about a region or area. The pivot portions 5808 and 5810 can include, for example, pivot pin portions 5812 located on paddle 5802 and pivot ring portions 5816 located near the base region of adjustable width paddle frames 5804 and 5806. In some implementations, the arrangement can be reversed with pivot pin portions 5812 located on paddle 5802 and pivot ring portions 5816 located on adjustable width paddle frames 5804 and 5806. As will be described further below, hinge or pivot portions 5808 and 5810 allow adjustable width paddle frames 5804 and 5806 to move in a pivoting or hinging motion as they change from a narrowed to an expanded position.

In some implementations, hinge or pivot portions 5808 and 5810 can be positioned a distance approximately halfway along the longitudinal length of the outer paddle of paddle 5802 as shown in FIG. 58. However, in some implementations, hinge or pivot portions 5808 and 5810 can be positioned along distances that are a quarter, third, half, two-thirds, three-quarters, etc., along the longitudinal length of the paddle structure (e.g., outer paddle structure). Iin the implementation shown, hinge or pivot portions 5808 and 5810 are arranged in a plane that is angularly offset from the plane of the outer paddle structure of paddle 5702. In some implementations, as shown in FIGS. 57A and 57B, hinge or pivot portions 5808 and 5810 can be arranged co-planar, substantially co-planar, and/or in a plane that is parallel and offset with the plane of the outer paddle structure of paddle 5802.

In some implementations, adjustable width paddle frames 5804 and 5806 include a distal connection portion 5822 in the form of a tab or cross-brace beam or element. Distal connection portion 5822 functions in the same manner as other distal connection portions described herein (e.g., see reference number 39670 (FIG. 43), reference number 49671 (FIG. 44), etc., and associated text) to effect movement of adjustable width paddle frames 5804 and 5806. Distal connection portion 5822 can include, for example, one or more portions for connecting to an actuation connector 5724 that can move longitudinally. In some implementations, connection portion 5822 can include an aperture, slot, flare, recess, cutout, loop, hook, etc. or any other implementation described herein. Distal connection portion 5822 can be connected to actuation connector 5724 via any of the described examples herein including, for example, wire, suture, sleeve, etc.

In operation, adjustable width paddle frames 5804 and 5806 can be shape set to the narrowed configuration shown in FIG. 58. Downward movement, as represented by arrow 5826, of actuation connector 5724 causes downward movement of connection portion 5822. This unwinds or partially unwinds and/or straightens or partially straightens a curved connection portion 5820 of the adjustable width paddle frames 5804 and 5806. This unwinding or straightening causes the adjustable width paddle frames 5804 and 5806 to pivot or rotate about hinge or pivot portions 5808 and 5810 as indicated generally by arrow 5828, which moves adjustable width paddle frames 5804 and 5806 outward. This pivoting or rotational movement causes adjustable width paddle frames 5804 and 5806 to change from their narrowed to their expanded position where they are extended or pivoted further away from the body of implantable device 5800. This arrangement can be referred to as actively expanding paddle frames, since the paddles can be shape set in the narrowed state and will only move to the expanded state when the pulling force 5826 is applied. In some implementations, the actively expanding paddle frames can control the force applied by the expanding paddle frames to the native valve leaflets, since the force applied to the leaflets is dependent on the applied pulling force 5826.

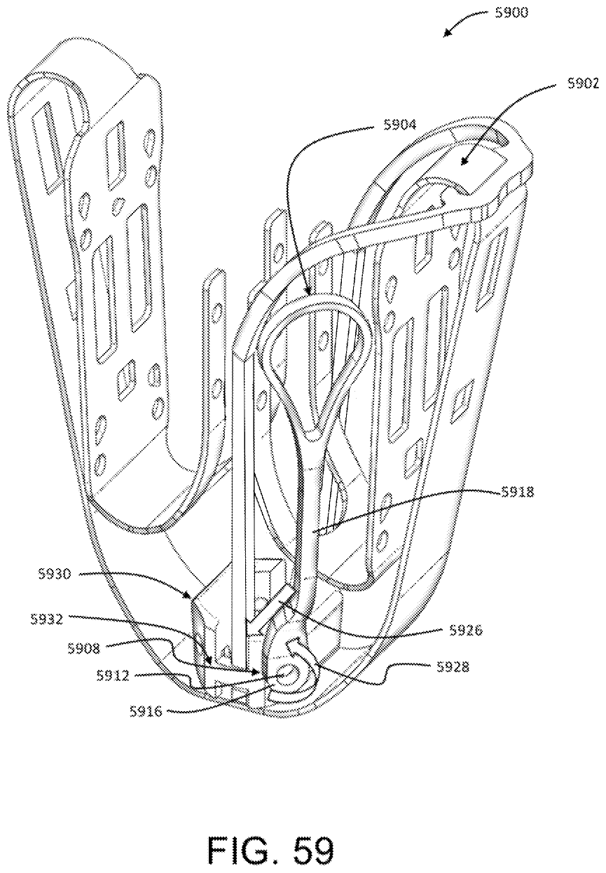

Referring now to FIG. 59, an implantable device 5900 having a pivot or hinged connection for the adjustable width paddle frame 5904 is shown. Similar to FIGS. 57A-57B, the paddles and paddle frames have been shown in isolation from other components of the device (e.g., central bushing, cap, retaining element, actuation elements, etc.), which can be found throughout the disclosure including in, for example, FIGS. 22-23, 26-34, and 38-56, and the associated text. Implantable device 5900 includes a paddle 5902 and first and second adjustable width paddle frames similar to the previous implementations, except only adjustable width paddle frame 5904 is presently shown. A similar set of paddle frames are present on other side(s) of retention portion 5930 and not shown but with the understanding that the present disclosure is equally applicable.

In some implementations, adjustable width paddle frame 5904 can include a proximal loop portion, pivot ring portion 5916 and intermediate strut or beam 5918. Other examples shown herein can also be used. In some implementations, adjustable width paddle frame 5904 can include hinge or pivot joint portion 5908 that allows movement of adjustable width paddle frame 5904. A similar hinge or pivot portion can be included for the other adjustable width paddle frames that are not shown in this view.

As previously mentioned, paddle frame movement can be a pivoting or hinge movement about a region or area. The pivot portions (e.g., 5908) can include, for example, pivot pin portions (e.g., 5912) located on retention portion 5930, which can be an independent component or part of a cap assembly, and pivot ring portions (e.g., 5916) located near the base region of the adjustable width paddle frames (e.g., 5904). In some implementations, the arrangement can be reversed with pivot pin portions 5912 located on retention portion 5930 and pivot pin portions 5912 located on adjustable width paddle frames 5904. As will be described further below, hinge or pivot portions 5908 allow adjustable width paddle frames 5904 to move in a pivoting or hinging motion as they change from a narrowed to an expanded position. Hinge or pivot portions 5908 can be positioned distally on the device and, in this implementation, on retention portion 5730. In some implementations, hinge or pivot portions 5808 are arranged in a plane that is angularly offset from the planar wall 5932 of the retention portion 5930. In some implementations, such as that shown in FIGS. 57A and 57B, hinge or pivot portions 5808 and 5810 can be arranged co-planar, substantially co-planar, and/or in a plane that is parallel and offset with respect to the planar wall 5932 of the retention portion 5930.

In operation, adjustable width paddle frames 5904 can be initially set to the narrowed configuration shown in FIG. 59. Downward movement as represented by arrow 5926 causes downward movement of the adjustable width paddle frame. The downward movement can be effected by any of the implementations described herein including actuation elements, connectors, sutures, wires, or other structures that can exert a pulling or downward force on adjustable paddle frame 5904. This expands adjustable width paddle frames 5904 outward and causes adjustable width paddle frames 5804 to pivot or rotate about hinge or pivot portions 5908 as indicated generally by arrow 5928. This pivoting or rotational movement can cause adjustable width paddle frames 5904 to move from their narrowed to their expanded position where they are extended or pivoted further away from the body of implantable device 5900. This arrangement can be referred to as actively expanding paddle frames, since the paddles can be shape set in the narrowed state and will only move to the expanded state when the pulling force 5826 is applied.

In some implementations, the actively expanding paddle frames can control the force applied by the expanding paddle frames to the native valve leaflets, since the force applied to the leaflets is dependent on the applied pulling force. In this example, the hinge or pivot portions 5908 are positioned on retention portion 5930 instead of paddle 5902 and, thus, any forces involved in this regard are substantially isolated from paddle 5902 and directed to retention portion 5930.

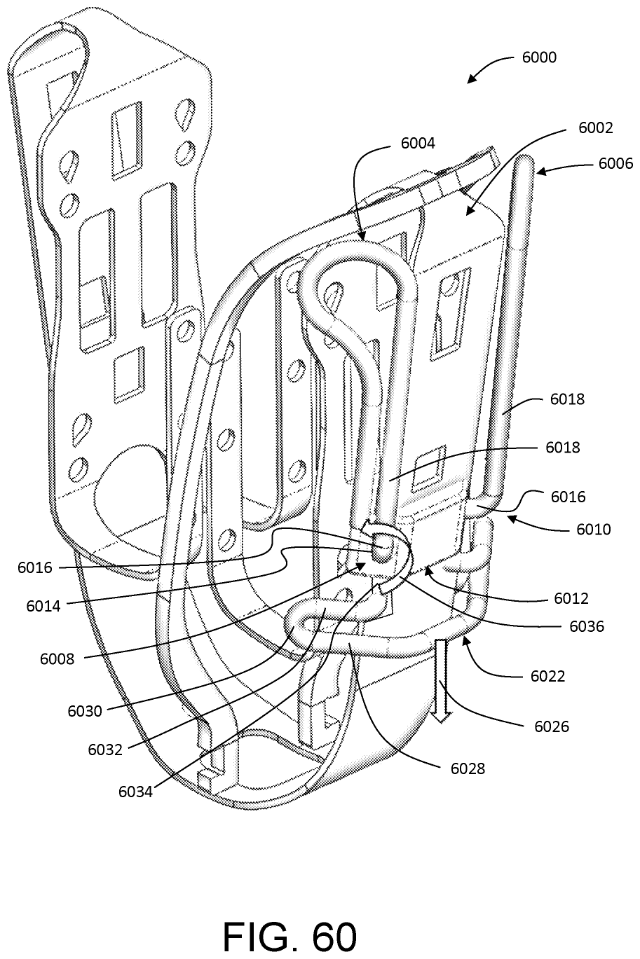

FIG. 60 illustrates an implementation of an implantable device 6000 having a pivot or hinged connection for the adjustable width paddle frames 6004 and 6006. Similar to FIGS. 57A-57B, the paddles and paddle frames have been shown in isolation from other components of the device (e.g., central bushing, cap, retaining element, actuation elements, etc.), which can be found throughout the disclosure including in, for example, FIGS. 22-23, 26-34, and 38-56, and the associated text. A similar set of paddle frames are present on the second paddle and not shown but with the understanding that the present disclosure is equally applicable.

Implantable device 6000 includes a paddle 6002 and first and second adjustable width paddle frames 6004 and 6006. Also included are hinge or pivot portions 6008 and 6010 that allow movement of adjustable width paddle frames 6004 and 6006. In some implementations, the movement is a bending, flexing, and or twisting type of pivoting or hinge movement about a region or area. The pivot joint portions 6008 and 6010 can include, for example, a guide portion 6014. In some implementations, the guide portion can be part of a rim, tube, passageway or other structure. As will be described further below, hinge or pivot portions 6008 and 6010 allow adjustable width paddle frames 6004 and 6006 to move in a pivoting or hinging motion by bending and/or flexing as the adjustable width paddle frames move from a narrowed to an expanded position.

In some implementations, hinge or pivot portions 6008 and 6010 can be part of a mount 6012 positioned a distance approximately halfway along the longitudinal length of the outer paddle of paddle 6002 as shown in FIG. 60. However, in some implementations, hinge or pivot portions 6008 and 6010 can be positioned along distances that are a quarter, third, half, two-thirds, three-quarters, etc., along the longitudinal length of the paddle structure (e.g., outer paddle structure). As shown in FIG. 60, hinge or pivot portions 6008 and 6010 are arranged in a plane that is angularly offset from the plane of the outer paddle structure of paddle 6002. In some implementations, as shown in FIGS. 57A and 57B, hinge or pivot portions 6008 and 6010 can be arranged co-planar, substantially co-planar, and/or in a plane parallel offset with the plane of the outer paddle structure of paddle 6002.

In some implementations, adjustable width paddle frames 6004 and 6006 include a distal connection portion 6022 in the form of a tab or cross-brace beam or element. Distal connection portion 6022 can function in the same manner as other distal connection portions described herein (e.g., see reference number 39670 (FIG. 43), reference number 49671 (FIG. 44), etc., and associated text) to effect movement of adjustable width paddle frames 6004 and 6006. In some implementations, distal connection portion 6022 can include, for example, one or more portions for connecting to an actuation connector 5724 (e.g., see FIG. 57) that can move longitudinally. In some implementations, connection portion 6022 can include an aperture, slot, flare, recess, cutout, loop, hook, etc. or any other implementation described herein. Distal connection portion 6022 can be connected to actuation connector 5724 via any of the described examples herein including, for example, wire, suture, sleeve, etc.

In some implementations, mount 6012 includes a guide portion 6014 for each adjustable width paddle frame 6004 and 6006. In such implementations, adjustable width paddle frames 6004 and 6006 can be shape set to the narrowed or collapse configuration shown in FIG. 60 and pass through internal passageways (not shown) in mount 6012. In some implementations, adjustable width paddle frames 6004 and 6006 include a plurality of beams or struts including 6018, 6016, 6028, 6030, 6032, 6032 and 6034, which can include one or more curved and linear portions (as shown).

In operation, adjustable width paddle frames 6004 and 6006 can be shape set to the narrowed configuration shown in FIG. 60. Adjustable width paddle frames 6004 and 6006 can be made of a shape-settable material such as, for example, nitinol or other similar material that can be set to a particular shape but also bent or flexed. Downward movement as represented by arrow 6026 of actuation connector 5724 (FIG. 57) causes downward movement of connection portion 6022. This movement draws beam or strut portion 6016 into mount 6012. Beam or strut portion 6016 includes a curved portion outside of guide portion 6014 of mount 6012. In some implementations, guide portion 6014 can be, for example, in the form of a bore or passageway having a rigid exterior (or other similar configuration that can act as a guide or guide rail).

As the curved portion of beam or strut 6016 encounters rigid guide portion 6014, it begins to conform to the guide portion 6014 and thus begins to unbend or lessen in curvature. This causes adjustable width paddle frames 6004 and 6006 to bend, flex, or twist outwardly thus pivoting or rotating about hinge or pivot portions 6008 and 6010, as indicated generally by arrow 6036. This pivoting or rotational movement causes adjustable width paddle frames 6004 and 6006 to move from their narrowed to their expanded position where they are extended or pivoted further away from the body of implantable device 6000.

Hence, the pivoting or hinging effect of the adjustable width paddle frames can be accomplished via an anchored guide portion and flexing or unflexing of the portion of the adjustable width paddle frames 6004 and 6006 via guide rail type arrangement. This arrangement can be referred to as actively expanding paddle frames, since the paddles can be shape set in the narrowed state and will only move to the expanded state when the pulling force 6026 is applied. In some implementations, the actively expanding paddle frames can control the force applied by the expanding paddle frames to the native valve leaflets, since the force applied to the leaflets depends on the applied pulling force 6026.

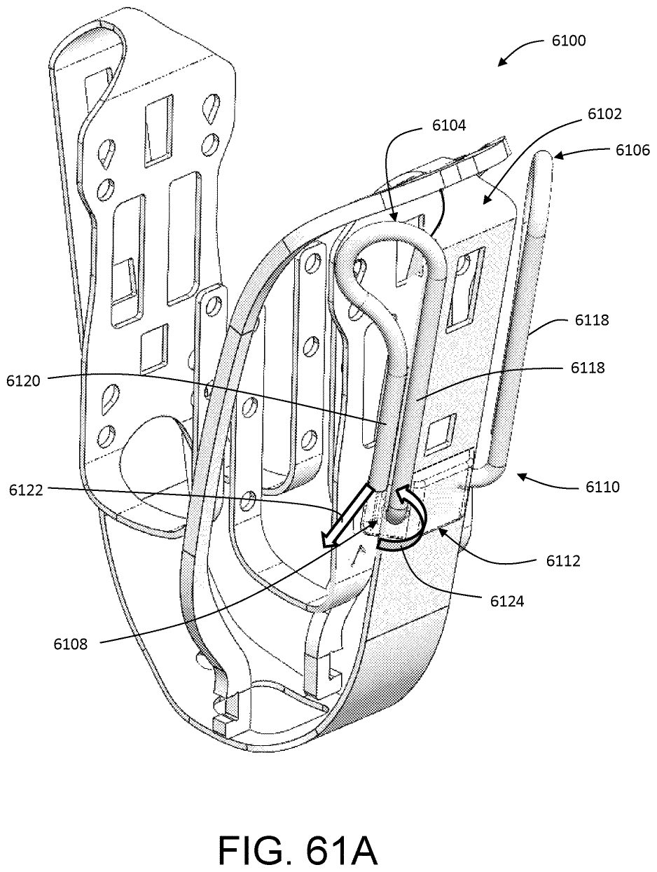

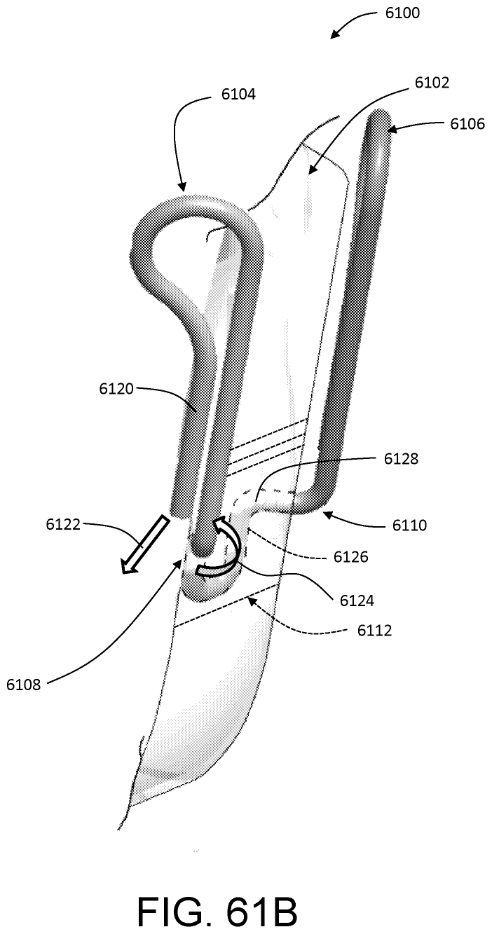

FIGS. 61A and 61B illustrate an implementation of an implantable device 6100 that is similar to FIG. 60, except that a distal connection portion 6022 is not used. Implantable device 6100 includes a paddle 6102 and first and second adjustable width paddle frames 6104 and 6106. Implantable device 6100 includes hinge or pivot joint portions 6108 and 6110 that allow movement of adjustable width paddle frames 6104 and 6106. In this implementation, the movement is a bending, flexing, and/or twisting type of pivoting or hinge movement about a region or area. The pivot portions 6108 and 6110 can include, for example, a guide portion (similar to guide portion 6014 of FIG. 60) that can be part of a rim, tube, passageway or other structure. As will be described further below, hinge or pivot portions 6108 and 6110 allow adjustable width paddle frames 6104 and 6106 to move in a pivoting or hinging motion by bending and/or flexing as they change from a narrowed to an expanded position.

In some implementations, hinge or pivot portions 6108 and 6110 can be part of or connected to a mount 6112 positioned a distance approximately halfway along the longitudinal length of the outer paddle of paddle 6102 as shown in FIGS. 61A and 61B. In other implementations, hinge or pivot portions 6108 and 6110 can be positioned along distances that are a quarter, third, half, two-thirds, three-quarters, etc., along the longitudinal length of the paddle structure (e.g., outer paddle structure). Also, in the implementation shown, hinge or pivot portions 6108 and 6110 are arranged in a plane that is angularly offset from the plane of the outer paddle structure of paddle 6102. Also, as shown in FIGS. 57A and 57B, hinge or pivot portions 6108 and 6110 can be arranged co-planar, substantially co-planar, and/or in a plane that is parallel to and offset with the plane of the outer paddle structure of paddle 6102.

Adjustable width paddle frames 6104 and 6106 can be shape set to the narrowed or collapse configuration as shown in FIGS. 61A and 61B. In some implementations, the adjustable width paddle frames 6104 and 6106 can have portions 6128 (FIG. 61B) that are located in internal spaces or passageways 6126 (FIG. 61B) in mount 6112. In some implementations, mount 6112 functions as an anchor or rigid connector mounting adjustable width paddle frames 6104 and 6106 to paddle 6102. Adjustable width paddle frames 6104 and 6106 can include a plurality of beams or struts including 6118 and 6120, which can be separated by a loop portion but also can otherwise be according to any paddle frame structure described herein. Beam or strut 6020 can be connected to an actuation element or connector (not shown), which can be via any of the implementations described herein.

In operation, adjustable width paddle frames 6104 and 6106 can be shape set to the narrowed configuration shown in FIGS. 61A and 61B. Adjustable width paddle frames 6104 and 6106 can be made of a shape-settable material such as, for example, nitinol or other similar material that can be set to a particular shape but also bent or flexed. Downward movement, as represented by arrow 6122, of beam or strut 6120 causes adjustable width paddle frames 6104 and 6106 to bend, flex, or twist outwardly thus pivoting or rotating about hinge or pivot portions 6108 and 6110, as indicated generally by arrow 6124. This pivoting or rotational movement causes adjustable width paddle frames 6104 and 6106 to move from their narrowed to their expanded position where they are extended or pivoted further away from the body of implantable device 6000.

Hence, the pivoting or hinging effect can be accomplished via an anchored mount portion and flexing or unflexing of portions of the adjustable width paddle frames 6104 and 6106. This arrangement can be referred to as actively expanding paddle frames, since the paddles can be shape set in the narrowed state and will only move to the expanded state when the pulling force 6122 is applied. In some implementations, the actively expanding paddle frames can control the force applied by the expanding paddle frames to the native valve leaflets, since the force applied to the leaflets is dependent on the applied pulling force 6122.

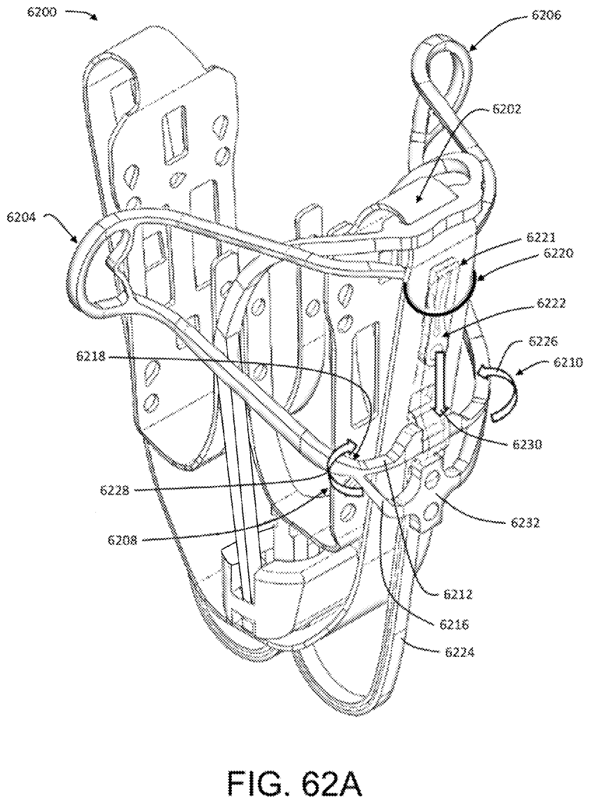

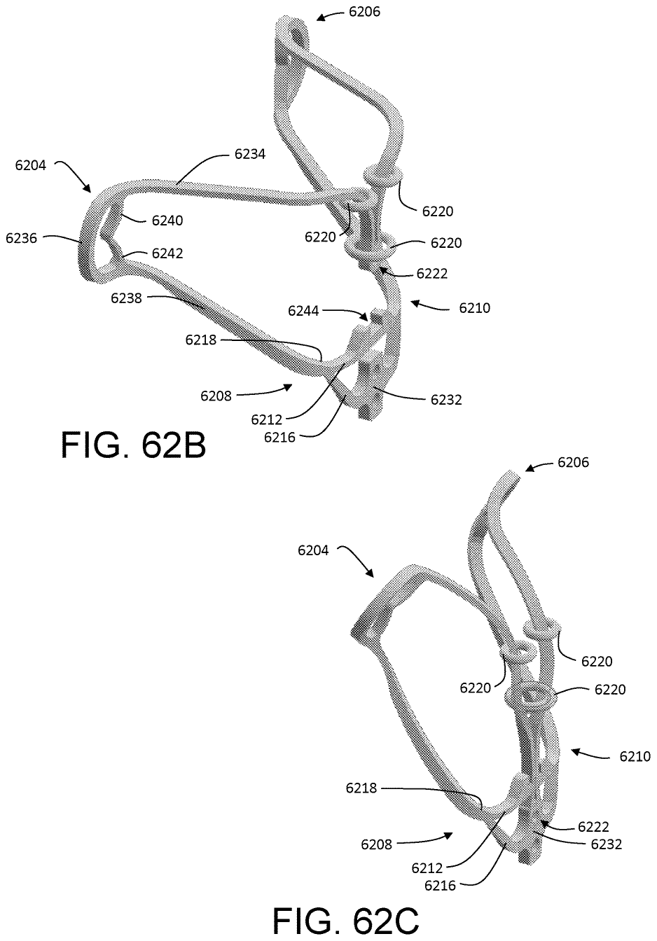

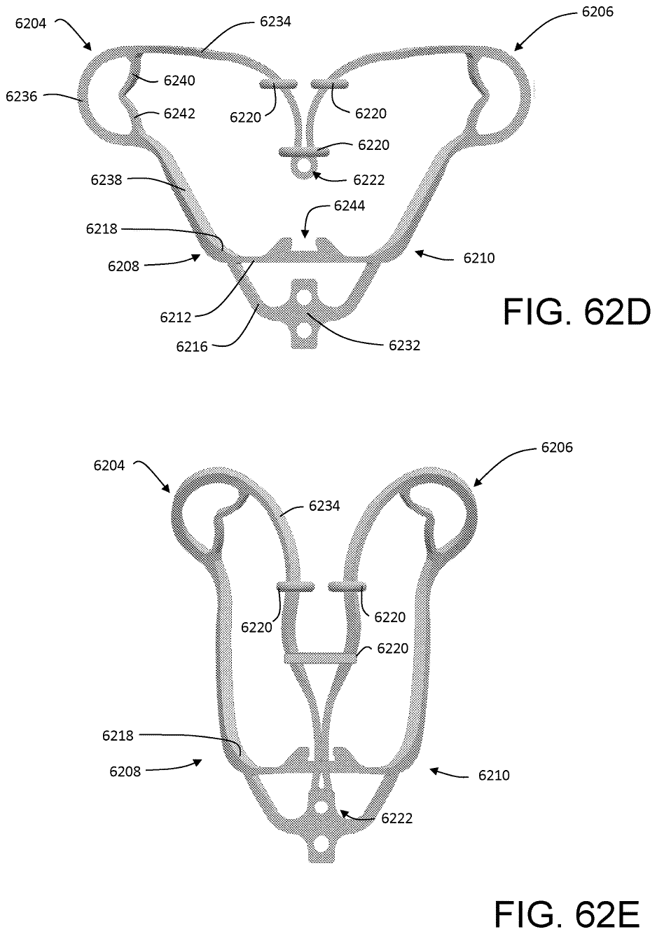

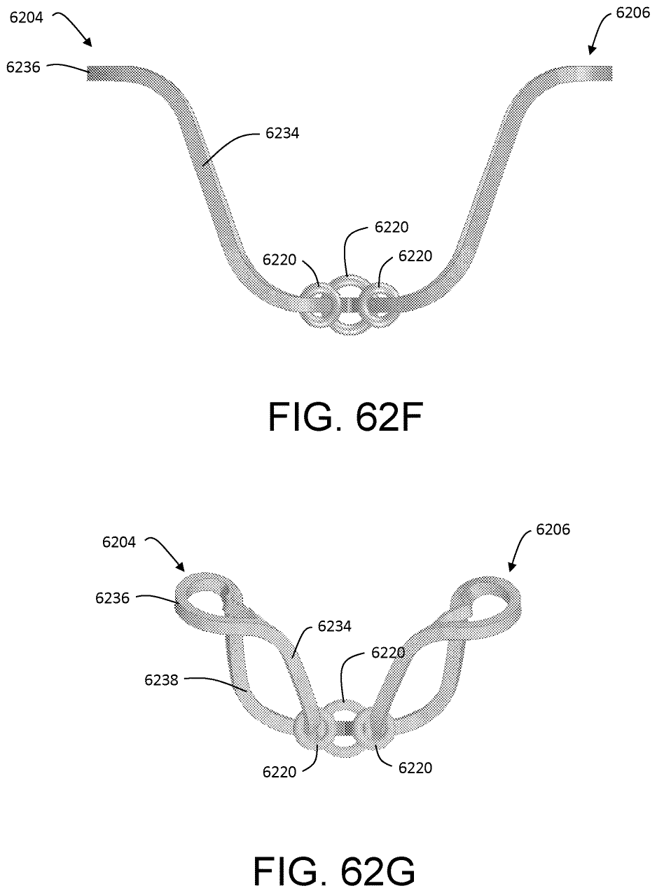

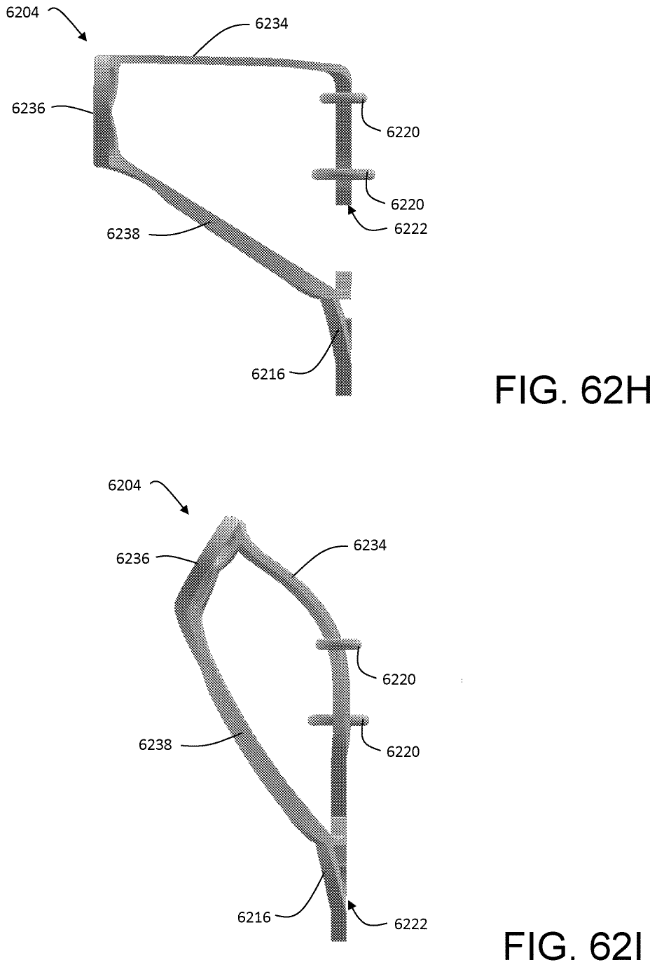

FIGS. 62A-62I illustrate an implementation of an implantable device 6200 having a pivot or hinged connection for the adjustable width paddle frames 6204 and 6206. Similar to FIGS. 57A-57B, the paddles and paddle frames have been shown in isolation from other components of the device (e.g., central bushing, cap, retaining element, actuation elements, etc.), which can be found throughout the disclosure including in, for example, FIGS. 22-23, 26-34, and 38-56, and the associated text. A similar set of paddle frames are present on the second paddle and not shown but with the understanding that the present disclosure is equally applicable.

Implantable device 6200 includes a paddle 6202 and first and second adjustable width paddle frames 6204 and 6206. Also included are hinge or pivot joint portions 6208 and 6210 that allow movement of adjustable width paddle frames 6204 and 6206. In this implementation, the movement is a bending, flexing, and/or twisting type of pivoting or hinge movement about a region or area. As will be described further below, hinge or pivot portions 6208 and 6210 allow adjustable width paddle frames 6204 and 6206 to move in a pivoting or hinging motion by bending and/or flexing as they move from a narrowed to an expanded position.

In some implementations, hinge or pivot portions 6208 and 6210 can be part of adjustable width paddle frames 6204 and 6206 in the form of bendable or flexible beam or strut portions and/or interconnections. In some implementations, the hinge or pivot portions 6208 and 6210 can be positioned a distance approximately halfway along the longitudinal length of the outer paddle of paddle 6202 as shown in FIGS. 62A-62I. In some implementations, hinge or pivot portions 6208 and 6210 can be positioned along distances that are a quarter, third, half, two-thirds, three-quarters, etc., along the longitudinal length of the paddle structure (e.g., outer paddle structure). In some implementations, hinge or pivot portions 6208 and 6210 are arranged in an offset plane or otherwise slightly angled or curved perspective from the plane of the outer paddle structure of paddle 6202. In some implementations, as shown in FIGS. 57A and 57B, hinge or pivot portions 6208 and 6210 can be arranged co-planar, substantially co-planar, and/or in a plane parallel offset with the plane of the outer paddle structure of paddle 6202.

Adjustable width paddle frames 6204 and 6206 can be shape set to the expanded configuration shown, but other configurations are also possible. In some implementations, a mount portion 6232 functions to connect and anchor a lower portion of adjustable width paddle frames 6204 and 6206 to paddle 6202. This arrangement can be referred to as passively expanding paddle frames, since the paddles can be shape set in the expanded state and will return to the expanded state in the absence of the pulling force 6230.

In some implementations, Adjustable width paddle frames 6204 and 6206 include a plurality of beams or struts. Adjustable width paddle frame 6204 will now be described with the understanding that adjustable width paddle frame 6206 is similarly constructed. Adjustable width paddle frames 6204 can include beams or struts 6212 and 6216 at its lower portion. Struts 6212 can be connected to mount portion 6232 at one end and to struts 6216 at its other end via connection 6218. Beam or strut 6238 can be connected at one end to connection 6218 and to curved strut 6236 at its other end. Curved strut 6236 can be connected at its other end to strut 6234, which can be connected to distal connection portion 6222. Between strut 6234 and strut 6238 one or more brace struts 6240 and 6244 are connected. While the beams or struts are illustrated with the arrangement of shapes shown in FIGS. 62A-62I, modifications thereto can also be made including more or less curvature, longer or shorter lengths and widths, etc. In some implementations, one or more guides 6220 can also be included and can be according to any of the implementations described herein.

Referring specifically to FIG. 62A, lower strut 6216 is connected to mount portion 6232, which is fastened or otherwise mounted, connected, or affixed to paddle 6202. Distal connection portion 6222, which can be according to any of the implementations described herein, is connected to an actuation connector 6224, which can also be according to any of the implementations described herein.

In operation, adjustable width paddle frames 6204 and 6206 can be shape set to the expanded configuration shown in FIGS. 62A, 62B, 62D, 62F, and 62H. FIGS. 62C, 62E, 62G, 62I show the narrowed configuration corresponding to FIGS. 62A and 62B, 62D, 62F, and 62H, respectively. Adjustable width paddle frames 6204 and 6206 can be made of a shape-settable material such as, for example, nitinol or other similar material that can be set to a particular shape but also bent or flexed. Downward movement, as represented by arrow 6230 of distal connection portion 6222 causes adjustable width paddle frames 6204 and 6206 to bend, flex, or twist inwardly thus pivoting or rotating about hinge or pivot portions 6208 and 6210, as indicated generally by arrows 6226, 6228. See also FIGS. 62C, 62E, 62G, 62I showing the narrowed or collapsed configuration. This pivoting or rotational movement causes adjustable width paddle frames 6204 and 6206 to move from their expanded to their narrowed position where they are drawn closer to the body of implantable device 6200. Hence, the pivoting or hinging effect can be accomplished via beams or struts and flexing or unflexing portions thereof to change the configuration of the adjustable width paddle frames 6204 and 6206.

For example, referring to FIGS. 62D and 62E, the shape-set expanded configuration (FIG. 62D) and narrowed configuration (62E) are shown. The change from the expanded configuration (FIG. 62D) to narrowed configuration (62E) is accomplished by pulling distal connection portion 6222 downward (as shown by arrow 6230 in FIG. 62A). The one or more guides 6220 are fixed in position via paddle 6202 and cause adjustable width paddle frames 6204 and 6206 to bend or flex about hinge or pivot portions 6208 and 6210. During this movement, struts 6212 and 6216 act as braces and resist movement while strut 6238 begins to bend or flex at connection 6218 resulting in the narrowed configuration shown in FIGS. 62C, 62E, 62G, 62I. One or more brace struts 6240 and 6242 provide additional rigidity to curved strut 6236 during this movement to minimize its bending or deformation.

In some implementations, strut 6212 includes an optional extension having a slot or cutout between two tabs for allowing portions of distal connection portion 6222 to pass therethrough. The optional extension can be used in connection with a hook, loop or other fastening device to further connect the lower portion of adjustable width paddle frames 6204 and 6206 to paddle 6202. In some implementations, paddle 6202 includes an aperture or window 6221 (FIG. 62A) through which distal connection portion 6222 can extend from one side of the outer paddle to the other to allow the pulling action of actuation connector 6224. Thus, by pulling on the top portions of adjustable width paddle frames 6204 and 6206, pivoting or hinging movement can be accomplished via bending or flexing action of the beams or struts. This arrangement can be referred to as actively expanding paddle frames, since the paddles can be shape set in the narrowed state and will only move to the expanded state when the pulling force 6230 is applied. In some implementations, the actively expanding paddle frames can control the force applied by the expanding paddle frames to the native valve leaflets, since the force applied to the leaflets is dependent on the applied pulling force 6230.

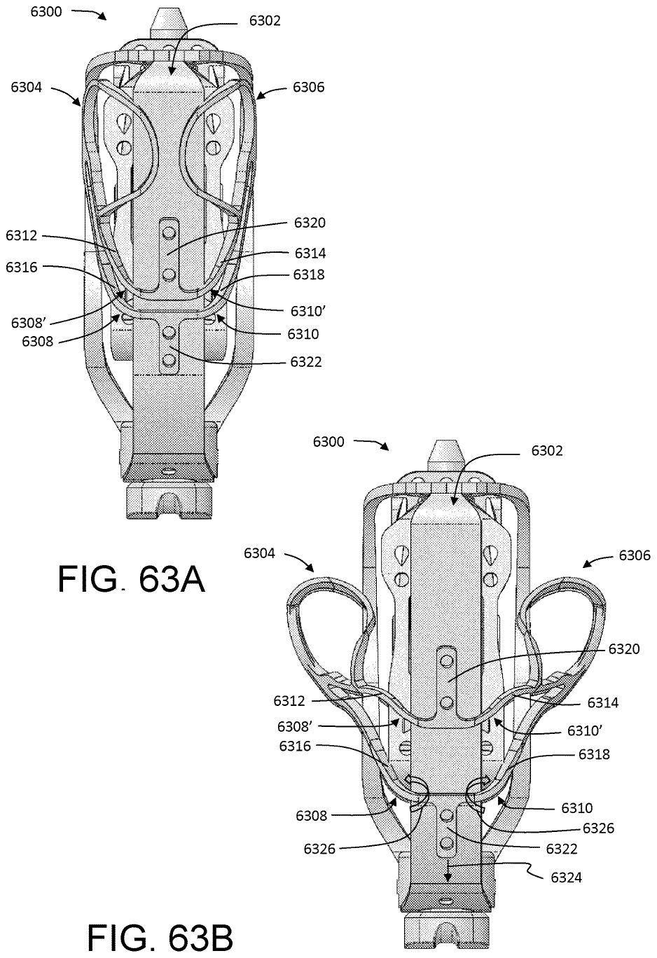

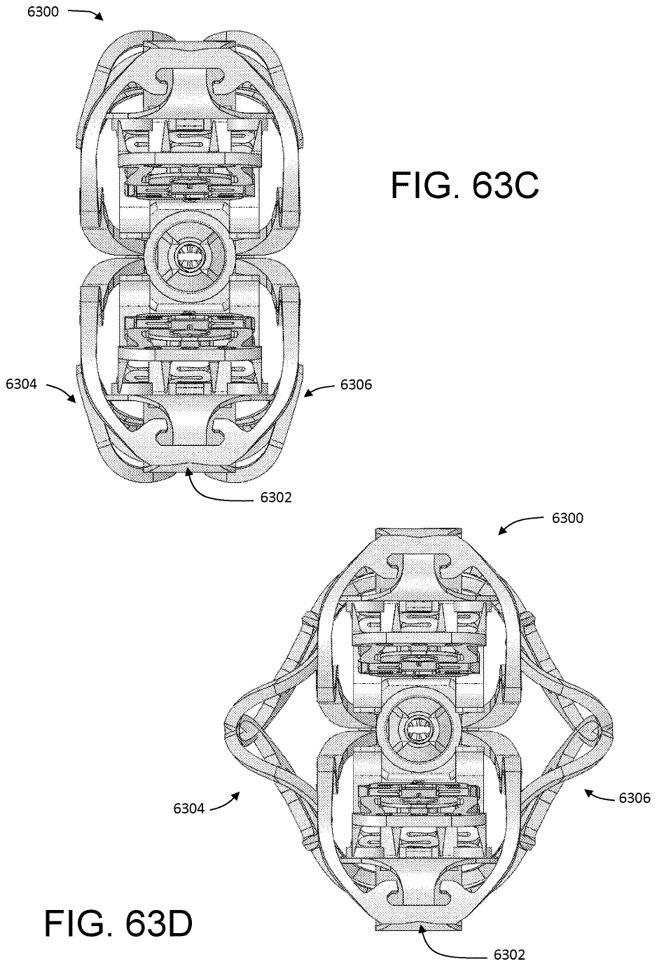

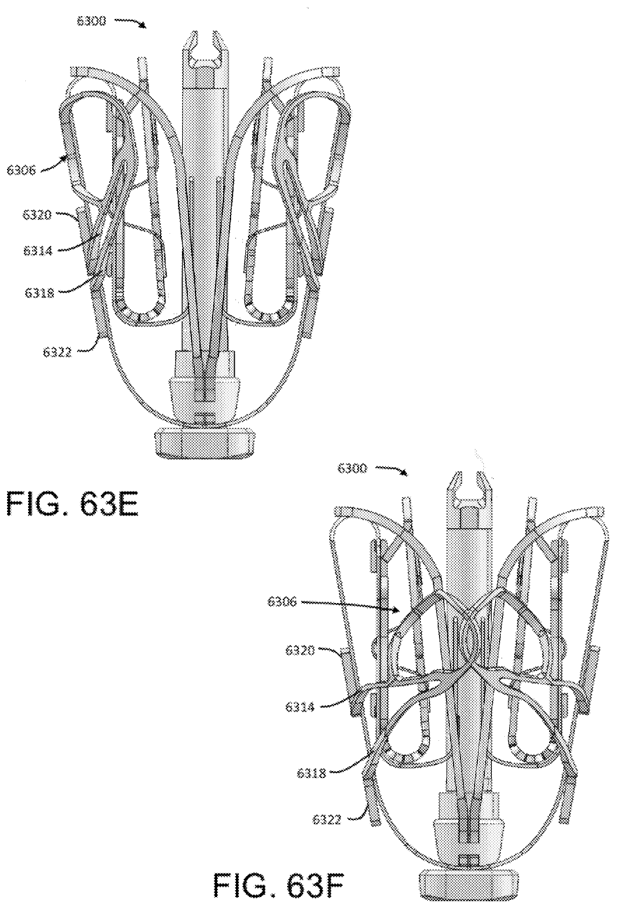

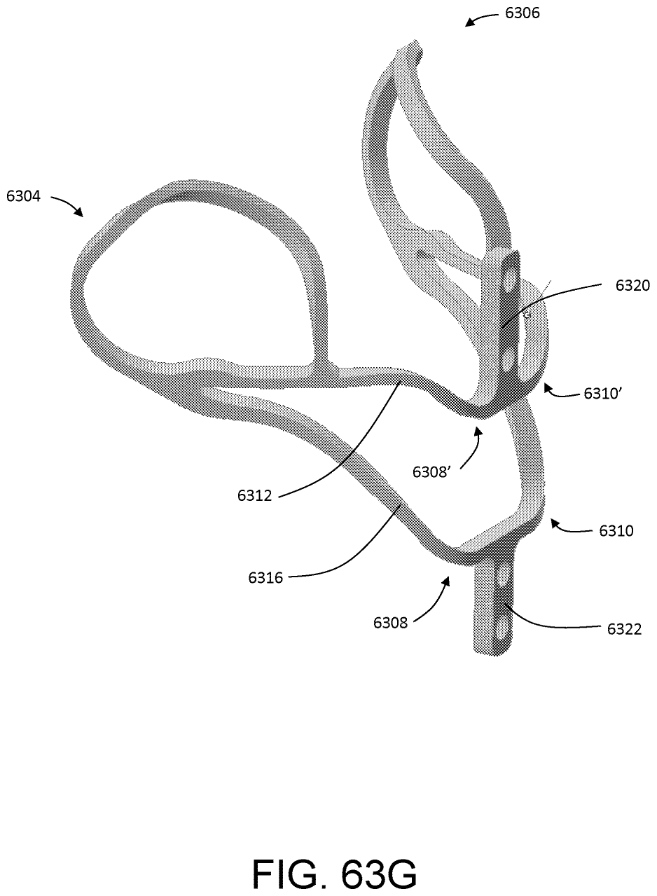

Referring now to FIGS. 63A-63G, an implementation of an implantable device 6300 having a pivot or hinged connection for the adjustable width paddle frames 6304 and 6306 is shown. In some implementations, each pivot or hinge portion includes first and second portions. For example, one pivot or hinge portion can be provided by pivot or hinge joint portions 6308 and 6308′ and another pivot or hinge portion is provided by 6310 and 6310′. As with previous descriptions, only one side will be described in detail with the understanding that the description is equally applicable to other paddle and paddle frame arrangement on implantable device 6300.

Implantable device 6300 includes a paddle 6302 and first and second adjustable width paddle frames 6304 and 6306. The included hinge or pivot portions 6308 and 6308′ and 6310 and 6310′ allow movement of adjustable width paddle frames 6304 and 6306 from the narrowed (FIGS. 63A, 63C, 63E) to the expanded configuration (FIGS. 63B, 63D, 63E, 63G, respectively). In some implementations, the movement is a bending, flexing, and or twisting type of pivoting or hinge movement about a region or area. As will be described further below, hinge or pivot portions 6308 and 6308′ and 6310 and 6310′ allow adjustable width paddle frames 6204 and 6006 to move in a pivoting or hinging motion by bending and/or flexing as they move from a narrowed to an expanded position.

In some implementations, hinge or pivot portions 6308 and 6308′ and 6310 and 6310′ can be part of adjustable width paddle frames 6204 and 6206 in the form of bendable or flexible beam or strut portions and/or interconnections. The hinge or pivot portions 6308 and 6308′ and 6310 and 6310′ can be positioned a distance approximately halfway along the longitudinal length of the outer paddle of paddle 6302 as shown in FIG. 63A. In some implementations, hinge or pivot portions 6308 and 6308′ and 6310 and 6310′ can be positioned along distances that are a quarter, third, half, two-thirds, three-quarters, etc., along the longitudinal length of the paddle structure (e.g., outer paddle structure). In some implementations, hinge or pivot portions 6308 and 6308′ and 6310 and 6310′ are arranged in an offset plane or otherwise slightly angled or curved perspective from the plane of the outer paddle structure of paddle 6302. In some implementations, as shown in FIGS. 57A and 57B, hinge or pivot portions 6308 and 6308′ and 6310 and 6310′ can be arranged co-planar, substantially co-planar, and/or in a plane parallel offset with the plane of the outer paddle structure of paddle 6302.

Adjustable width paddle frames 6304 and 6306 can be shape set to the narrowed configuration shown, but other configurations are also possible. In some implementations, a mount portion 6320 functions to connect and anchor an upper portion and a lower portion of adjustable width paddle frames 6304 and 6306 to paddle 6302.

Adjustable width paddle frames 6304 and 6306 can include a plurality of beams or struts. Adjustable width paddle frame 6204 includes beams or struts 6312, 6314 at its mid to upper portion and struts 6316, 6318 at its lower portion. Struts 6212, 6314 are connected to mount portion 6320 at one end and to a loop portion of paddle frame 6304 at its other end. Mount portion 6320 connects and affixes the mid to upper portion of adjustable width paddle frames 6304 and 6306 to paddle 6302. Where struts 6312, 6314 connect to mount portion 6320, a flexible curved portion of the strut can be present. Struts 6316, 6318 can be connected to a distal connection portion 6322 also via a flexible curved strut portion. In some implementations, these flexible portions need not be curved or angled but can be linear or strait. While the beams or struts are illustrated with the arrangement of shapes shown in FIGS. 63A-63G, modifications thereto can also be made including more or less curvature, longer or shorter lengths and widths, etc. One or more guides can also be included and can be according to any of the implementations described herein.

Referring specifically to FIGS. 63A and 63B, lower strut 6316 is connected to distal connection portion 6322, which can be according to any of the implementations described herein. The distal connection portion 6322 can be connected to an actuation connector (e.g., see actuation connector 6224 in FIG. 62A), which can also be according to any of the implementations described herein.

In operation, adjustable width paddle frames 6304 and 6306 can be shape set to the narrowed configuration shown in FIGS. 63A, 63C, and 63E. FIGS. 63B, 63D, and 63F illustrate the corresponding expanded configurations for FIGS. 63A, 63C, and 63E, respectively. As with other examples, adjustable width paddle frames 6304 and 6306 can be made of a shape-settable material such as, for example, nitinol or other similar material that can be set to a particular shape but also bent or flexed. Downward movement, as represented by arrow 6324 in FIG. 63B of distal connection portion 6322 causes adjustable width paddle frames 6304 and 6306 to bend, flex, or twist outwardly thus pivoting or rotating about hinge or pivot portions 6308 and 6308′ and 6210, as indicated generally by arrows 6326. This pivoting or rotational movement causes adjustable width paddle frames 6304 and 6306 to move from their narrowed to their expanded position where they are extended further from the body of implantable device 6300. Hence, the pivoting or hinging effect can be accomplished via beams or struts and flexing or unflexing portions thereof to change the configuration of the adjustable width paddle frames 6204 and 6206.

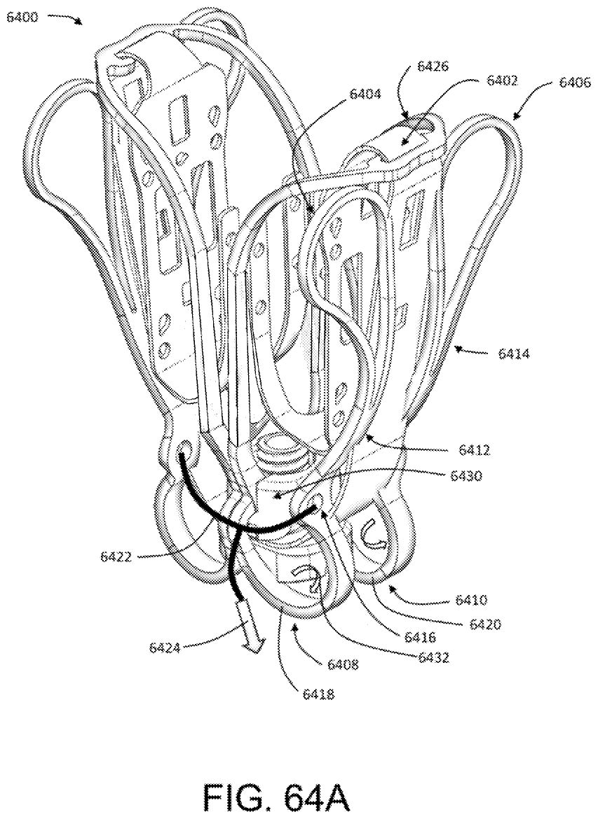

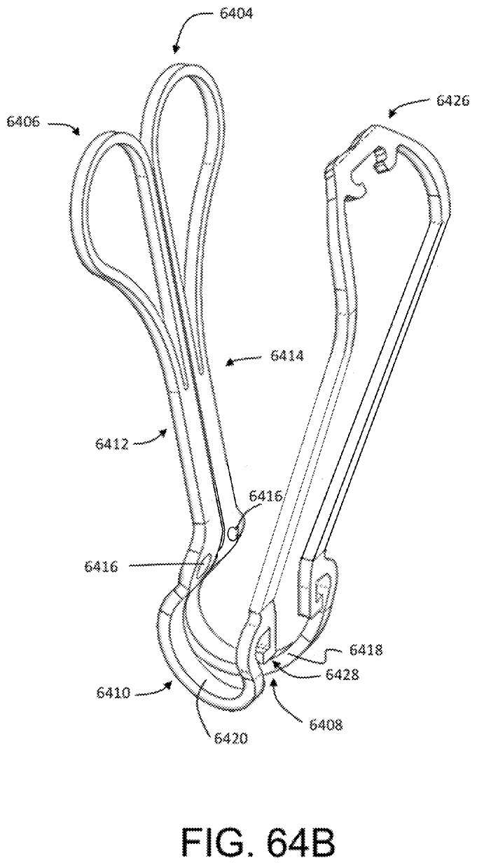

Referring now to FIGS. 64A and 64B, an implementation of an implantable device 6400 having a pivot or hinged connection for the adjustable width paddle frames 6404 and 6406 is shown. Similar to FIGS. 57A-57B, the paddles and paddle frames are shown in isolation from other components of the device (e.g., central bushing, cap, actuation elements, etc.), which can be found throughout the disclosure including in, for example, FIGS. 22-23, 26-34, and 38-56, and the associated text. A similar set of paddle frames are present on the second paddle with the understanding that the present disclosure is equally applicable.

Implantable device 6400 includes a paddle 6402 and first and second adjustable width paddle frames 6404 and 6406. Implantable device 6400 can include hinge or pivot joint portions 6408 and 6410 that allow movement of adjustable width paddle frames 6404 and 6406. In some implementations, the movement is a bending, flexing, and or twisting type of pivoting or hinge movement about a region or area. As will be described further below, hinge or pivot portions 6408 and 6410 allow adjustable width paddle frames 6404 and 6406 to move in a pivoting or hinging motion by bending and/or flexing as they move from a narrowed (as shown) to an expanded position. Hinge or pivot portions 6408 and 6410 can be part of adjustable width paddle frames 6404 and 6406 in the form of bendable or flexible beam or strut portions and/or interconnections. The hinge or pivot portions 6408 and 6410 can be positioned proximate a retention member 6430 (or cap assembly).

As shown in FIGS. 64A and 64B, hinge or pivot portions 6408 and 6410 can be formed and located near paddle frame mounting extension, clips or hooks 6428. Hinge or pivot portions 6408 and 6410 can include a plurality of beams or struts including 6418 and 6420, which can be connected to mounting extension, clips or hooks 6428 thereby anchoring hinge or pivot portions 6408 and 6410 to a location. In some implementations, apertures 6416 (or similar structures) can be included for connecting adjustable width paddle frames 6404 and 6406 to an actuation connector via devices 6422 (e.g., sutures, wires, hooks, etc.). Adjustable width paddle frames 6404 and 6406 can include loop portions connected to beams or struts 6412 and 6414 and a cantilever, spring or bias portion 6426 connected to the opposite paddle structure.

In operation, adjustable width paddle frames 6404 and 6406 can be shape set to the narrowed configuration shown in FIGS. 64A and 64B. As with other examples, adjustable width paddle frames 6404 and 6406 can be made of a shape-settable material such as, for example, nitinol or other similar material that can be set to a particular shape but also bent or flexed. Downward movement, as represented by arrow 6424 causes adjustable width paddle frames 6404 and 6406 to bend, flex, or twist outwardly about beam or struts 6418 and 6420. This results in pivoting or rotating movement about hinge or pivot portions 6408 and 6410, as indicated generally by arrow 6432. This pivoting or rotational movement causes adjustable width paddle frames 6404 and 6406 to move from their narrowed to their expanded position where they are extended further from the body of implantable device 6400.

Hence, the pivoting or hinging effect can be accomplished via beams or struts and flexing or unflexing portions thereof to change the configuration of the adjustable width paddle frames 6404 and 6406. This arrangement can be referred to as actively expanding paddle frames, since the paddles can be shape set in the narrowed state and move to the expanded state when the pulling force 6424 is applied. In some implementations, the actively expanding paddle frames can control the force applied by the expanding paddle frames to the native valve leaflets, since the force applied to the leaflets is dependent on the applied pulling force 6424.

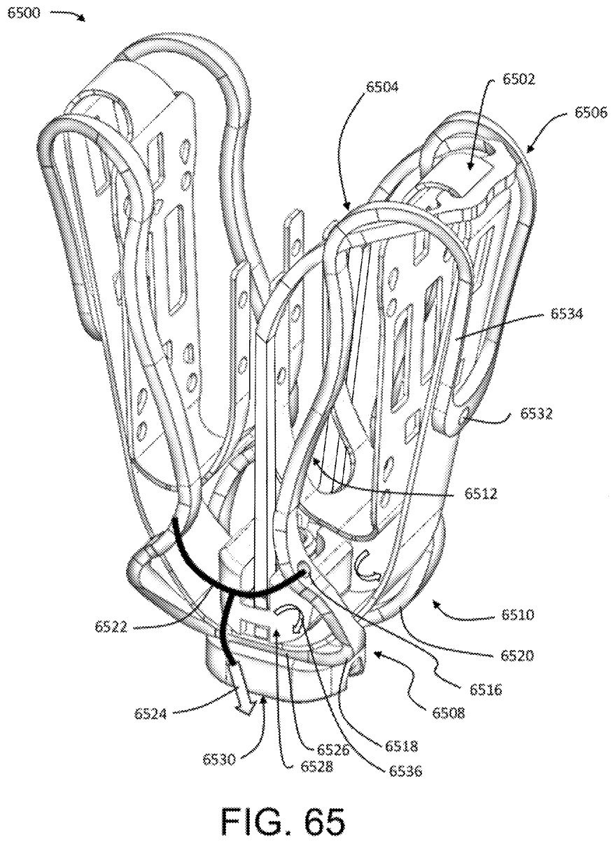

Referring now to FIG. 65, an implementation of an implantable device 6500 having a pivot or hinged connection for the adjustable width paddle frames 6504 and 6506 is shown. Similar to FIGS. 57A-57B, the paddles and paddle frames are shown in isolation from other components of the device (e.g., central bushing, actuation elements, etc.), which can be found throughout the disclosure including in, for example, FIGS. 22-23, 26-34, and 38-56, and the associated text. A similar set of paddle frames are present on the second paddle with the understanding that the present disclosure is equally applicable. In some implementations, the paddles are made from a single body that is laser cut and shape set to the geometry shown in FIG. 65. That is, all of the paddles'beams and struts are formed in a single body or piece of material.

Implantable device 6500 can include a paddle 6502 and first and second adjustable width paddle frames 6504 and 6506. Also included are hinge or pivot joint portions 6508 and 6510 that allow movement of adjustable width paddle frames 6504 and 6506. In some implementations, the movement is also a bending, flexing, and/or twisting type of pivoting or hinge movement about a region or area.

As will be described further below, hinge or pivot portions 6508 and 6510 allow adjustable width paddle frames 6504 and 6506 to move in a pivoting or hinging motion by bending and/or flexing as they change from a narrowed (as shown) to an expanded position. Hinge or pivot portions 6508 and 6510 can be part of adjustable width paddle frames 6504 and 6506 in the form of bendable or flexible beam or strut portions and/or interconnections. The hinge or pivot portions 6508 and 6510 can be positioned proximate a retention member 6528 (or cap 6530) and/or between retention member 6528 and cap 6530. Adjustable width paddle frames 6504 and 6506 can be retained in that location via a clamping, friction, snap-fit, interference fit, fastening, or any other suitable arrangement. Apertures 6516 (or similar structures) can also be included for connecting adjustable width paddle frames 6504 and 6508 to an actuation connector via devices 6522 (e.g., sutures, wires, hooks, etc.) Adjustable width paddle frames 6504 and 6506 can include beams or struts 6418 and 6420. Adjustable width paddle frames 6504 and 6506 can include struts 6512, 6526 and 6534 and are further connected or anchored to paddle 6502 via and a pin or pin-like element 6532 or other suitable fastener or connector. Holes or apertures 6516 (or similar structures) for connecting adjustable width paddle frames 6504 and 6508 to an actuation connector via devices 6522 (e.g., which can be sutures, wires, hooks, etc.).

In operation, adjustable width paddle frames 6504 and 6506 can be shape set to the narrowed configuration shown in FIG. 65. As with other examples, adjustable width paddle frames 6504 and 6506 can be made of a shape-settable material such as, for example, nitinol or other similar material that can be set to a particular shape but also bent or flexed. Downward movement, as represented by arrow 6524 causes adjustable width paddle frames 6504 and 6506 to bend, flex, or twist outwardly about beam or struts 6518 and 6520. Beams or struts 6518, 6520 also flex, bend and/or twist to allow for the expansion. This results in pivoting or rotating movement about hinge or pivot portions 6508 and 6510, as indicated generally by arrow 6536. This pivoting or rotational movement causes adjustable width paddle frames 6504 and 6506 to move from their narrowed to their expanded position where they are extended further from the body of implantable device 6500. Hence, the pivoting or hinging effect can be accomplished via beams or struts and flexing or unflexing portions thereof to change the configuration of the adjustable width paddle frames 6504 and 6506. This arrangement can be referred to as actively expanding paddle frames, since the paddles can be shape set in the narrowed state and move to the expanded state when the pulling force 6524 is applied. In some implementations, the actively expanding paddle frames can control the force applied by the expanding paddle frames to the native valve leaflets, since the force applied to the leaflets is dependent on the applied pulling force 6524.

As discussed above, some implementations can provide an active expansion allowing control of the pressure applied to leaflets. For example, in the implementations of FIGS. 58, 59, 60, 61A-61B, 63A-63G, 64A-64B and 65, the paddles are set to their narrow or collapsed position and use an actuating force to move the paddles to their expanded position. For example, in FIG. 58, downward movement of tab or connection portion 5822 causes paddles 5804 and 5806 to move outwardly to their expanded position via hinge or pivot portions 5808 and 5810. During leaflet capture, further movement of tab or connection portion 5822 allows for active control of the amount of pressure placed on the leaflets by the paddles. For example, further downward movement of connection portion 5822 actively places increasing pressure on the leaflets by the paddles. Lesser downward or upward movement (when the paddles are expanded) actively reduces or decreases the pressure on the leaflets by the paddles. Hence, the force of movement used to expand the paddles from their narrow or collapsed state or position also provides an active force control on the leaflets by the paddles.

Referring now to FIGS. 66A and 66B, an implementation of an implantable device 6600 having a pivot or hinged joint connection for the adjustable width paddle frames 6604 and 6606 is shown. A similar set of paddle frames are present on the second paddle with the understanding that the present disclosure is equally applicable. Implantable device 6600 includes a paddle 6602 and first and second adjustable width paddle frames 6604 and 6606. Implantable device 6600 can include hinge or pivot joint portions 6608 and 6610 that allow movement of adjustable width paddle frames 6504 and 6506. In this implementation, the movement is also a bending, flexing, and/or twisting type of pivoting or hinge movement about a region or area.

As will be described further below, hinge or pivot portions 6608 and 6610 allow adjustable width paddle frames 6604 and 6606 to move in a pivoting or hinging motion by bending and/or flexing as they change from an expanded (as shown) to a narrowed position. The narrowed position is indicated by 6604′ and 6606′. Hinge or pivot portions 6608 and 6610 can be part of adjustable width paddle frames 6604 and 6606 in the form of bendable or flexible beam or strut portions 6616 and/or interconnections. The hinge or pivot portions 6608 and 6610 can be positioned proximate a retention member 6628 (or cap 6630) and/or between retention member 6628 and cap 6630. Adjustable width paddle frames 6604 and 6606 can be retained in that location via a clamping, friction, snap-fit, interference fit, fastening, or any other suitable arrangement.

In some implementations, a distal connection portion 6622 is included for connecting adjustable width paddle frames 6604 and 6606 to actuation connector 5724. This connection can be according to any of the implementations or devices 6626 described herein including, for example, clips, hooks, wires, sutures, etc. As previously described, actuation connector 5724 can be shape set to provide a bias or preload. For example, actuation connector 5724 can be shape set to extend outwardly from implantable device 6600 as shown in FIG. 66B. This can provide, for example, a pre-load on actuation connector 5724. When connected to actuation connector 5724, distal connection portion 6622 is flexed inward toward actuation connector 5724 and forms the same orientation as distal connection portion 5722 shown in FIGS. 57A-57B.

One or more guides 6620 are also provided as shown and can also be according to any of the implementations described herein. Adjustable width paddle frames 6604 and 6606 include a plurality of beams or struts including struts 6612, 6614, and 6616. Struts 6612 can be located distally and can be secured to retention member 6628 via any of the implementations disclosed herein including, for example, hook, clip, snap fit, interference fit, fasteners, etc.

In operation, adjustable width paddle frames 6604 and 6606 can be shape set to the expanded configuration shown in FIGS. 66A and 66B. This arrangement can be referred to as passively expanding paddle frames, since the paddles can be shape set in the expanded state and will return to the expanded state in the absence of the pulling force.

As with other examples, adjustable width paddle frames 6604 and 6606 can be made of a shape-settable material such as, for example, nitinol or other similar material that can be set to a particular shape but also bent or flexed. Downward movement, as represented by arrow 6624 of distal connection portion 6622 causes adjustable width paddle frames 6604 and 6606 to bend, flex, or twist inwardly thus pivoting or rotating about hinge or pivot portions 6608 and 6610, as indicated generally by arrows 6632. This pivoting or rotational movement causes adjustable width paddle frames 6604 and 6606 to move from their expanded to their narrowed position 6604′ and 6606′ where they are drawn closer to the body of implantable device 6200. Hence, the pivoting or hinging effect can be accomplished via beams or struts and flexing or unflexing portions thereof to change the configuration of the adjustable width paddle frames 6604 and 6606.

The following includes additional description of examples. The examples are intended to illustrate examples of combinations of elements and are not intended to limit the scope of any particular example or implementation disclosed herein.

Example 1. A device useable for treating and/or repairing a native valve of a heart, the device comprising: one or more paddles movable between an open position and a closed position, wherein the one or more paddles are configured to attach the device to the native valve; one or more adjustable width frame members coupled to the one or more paddles such that the one or more adjustable width frame members open and close with the one or more paddles; one or more guides; wherein the one or more adjustable width frame members extend through the one or more guides; and wherein the one or more guides control a shape of the one or more adjustable width frame members as the one or more adjustable width frame members move between a narrow configuration and a wide configuration.

Example 2. The device of example 1 further comprising a connector coupled to the one or more adjustable width frame members configured to move the one or more adjustable width frame members between the narrow configuration and the wide configuration.

Example 3. The device of example 2 wherein the connector includes attachment portions that connect to the one or more adjustable width frame members.

Example 4. The device of example 1 further comprising one or more inner frame members coupled to the one or more paddles configured to bias the one or more paddles to the closed position.

Example 5. The device of example 1 wherein each of the one or more adjustable width frame members have an anterior portion extending from the medial side of the device to the lateral side of the device and a posterior portion extending from the medial side of the device to the lateral side of the device opposite the anterior portion.

Example 6. The device of example 5, wherein the anterior portion and the posterior portion of each adjustable width frame member attach to one, or both, of an inner frame portion and the one or more paddles at a proximal portion of the device.

Example 7. The device of any one of examples 1-6, wherein each of the one or more adjustable width frame members comprise a shape-memory alloy.

Example 8. The device of any one of examples 1-7, wherein the one or more adjustable width frame members are symmetric.

Example 9. The device of any one of examples 1-8 wherein each guide of the one or more guides comprises a sling.

Example 10. The device of any one of examples 1-8 wherein each guide of the one or more guides comprises a one or more loops.

Example 11. The device of example 1 wherein the one or more adjustable width frame members are attached to a proximal portion of the device.

Example 12. The device of any one of examples 1-10 wherein the one or more adjustable width frame members are attached to a distal portion of the device.

Example 13. The device of example 2 wherein the one or more adjustable width frame members are attached to a proximal portion of the device and the connector is pulled into a distal portion of the device to narrow the one or more adjustable width frame members.

Example 14. The device of example 2 wherein the one or more adjustable width frame members are attached to a distal portion of the device and the connector is pulled into a proximal portion of the device to narrow the one or more adjustable width frame members.

Example 15. A system useable for treating and/or repairing a native valve of a heart, the system comprising: a device comprising: one or more paddles; one or more adjustable width frame members coupled to the one or more paddles such that the one or more adjustable width frame members open and close with the one or more paddles; one or more guides; wherein the one or more adjustable width frame members extend through the one or more guides; wherein the one or more guides control a shape of the one or more adjustable width frame members as the one or more adjustable width frame members move between a narrow configuration and a wide configuration; a delivery device comprising: an opening and closing control element coupled to the one or more paddles configured to move the one or more paddles between an open position and a closed position; wherein the one or more paddles are configured to attach the device to the native valve by moving the one or more paddles to the closed position; and a width control element coupled to the one or more adjustable width frame members configured to move the one or more adjustable width frame members move between the narrow configuration and the wide configuration.

Example 16. The system of example 15 wherein the device further comprises a connector coupled to the one or more adjustable width frame members and the width control element configured to move the one or more adjustable width frame members between the narrow configuration and the wide configuration in response to movement by the width control element.

Example 17. The system of example 16 wherein the connector includes attachment portions that connect to the one or more adjustable width frame members.

Example 18. The system of example 15 wherein the device further comprises one or more inner frame members coupled to the one or more paddles configured to bias the one or more paddles to the closed position.

Example 19. The system of example 15 wherein each of the one or more adjustable width frame members have an anterior portion extending from the medial side of the device to the lateral side of the device and a posterior portion extending from the medial side of the device to the lateral side of the device opposite the anterior portion.

Example 20. The system of example 19, wherein the anterior portion and the posterior portion of each adjustable width frame member attach to one, or both, of an inner frame portion and the one or more paddles at a proximal portion of the device.

Example 21. The system of any one of examples 15-20, wherein each of the one or more adjustable width frame members comprise a shape-memory alloy.

Example 22. The system of any one of examples 15-21, wherein the one or more adjustable width frame members are symmetric.

Example 23. The system of any one of examples 15-22 wherein each guide of the one or more guides comprises a sling.

Example 24. The system of any one of examples 15-22 wherein each guide of the one or more guides comprises one or more loops.

Example 25. The system of any one of examples 15-24 wherein the one or more adjustable width frame members are attached to a proximal portion of the device.

Example 26. The system of any one of examples 15-24 wherein the one or more adjustable width frame members are attached to a distal portion of the device.

Example 27. The system of example 16 wherein the one or more adjustable width frame members are attached to a proximal portion of the device and the connector is pulled into a distal portion of the device to narrow the one or more adjustable width frame members.

Example 28. The system of example 16 wherein the one or more adjustable width frame members are attached to a distal portion of the device and the connector is pulled into a proximal portion of the device to narrow the one or more adjustable width frame members.

Example 29. A device usable for treating and/or repairing a native valve of a heart, the device comprising: a pair of paddles movable between an open position and a closed position, wherein the pair of paddles are configured to attach the device to the native valve; pair of adjustable width frame members coupled to the pair of paddles such that the pair of adjustable width frame members open and close with the pair of paddles; and wherein the pair of adjustable width frame members are attached to a distal portion of the device.

Example 30. The device of example 29 wherein the pair of adjustable width frame members are attached directly to a distal portion of a main body of the device.

Example 31. The device of example 30 further comprising a spacer disposed over the main body of the device.

Example 32. The device of example 29 wherein the pair of adjustable width frame members are attached directly to a cap of the device.

Example 33. The device of example 32 wherein movement of the cap moves the pair of paddles between the open position and the closed position.

Example 34. The device of any one of examples 29-33 further comprising a connector coupled to the pair of adjustable width frame members configured to move the pair of adjustable width frame members between a narrow configuration and a wide configuration.

Example 35. The device of example 34 wherein the connector is pulled into a proximal portion of the device to narrow the pair of adjustable width frame members.

Example 36. The device of example 35 wherein the connector is pulled into a collar of the device.

Example 37. The device of example 34 wherein the connector includes attachment portions that connect to the pair of adjustable width frame members.

Example 38. The device of any one of examples 29-37 further comprising a pair of inner frame members coupled to the pair of paddles configured to bias the pair of paddles to the closed position.

Example 39. The device of any one of examples 29-38 wherein each of the pair of adjustable width frame members have an anterior portion extending from the medial side of the device to the lateral side of the device and a posterior portion extending from the medial side of the device to the lateral side of the device opposite the anterior portion.

Example 40. The device of example 39, wherein the anterior portion and the posterior portion of each adjustable width frame member attach to one, or both, of an inner frame portion and the pair of paddles at a proximal portion of the device.

Example 41. The device of any one of examples 29-40, wherein each of the pair of adjustable width frame members comprise a shape-memory alloy.

Example 42. The device of any one of examples 29-41, wherein the pair of adjustable width frame members are symmetric.

Example 43. A system usable for treating and/or repairing a native valve of a heart, the system comprising: a device comprising: one or more paddles movable between an open position and a closed position, wherein the one or more paddles are configured to attach the device to the native valve; one or more adjustable width frame members coupled to the one or more paddles such that the one or more adjustable width frame members open and close with the one or more paddles; wherein the one or more adjustable width frame members are attached to a distal portion of the device; a delivery device comprising: an opening and closing control element coupled to the one or more paddles configured to move the one or more paddles between the open position and the closed position; wherein the one or more paddles are configured to attach the device to the native valve by moving the one or more paddles to the closed position; and a width control element coupled to the one or more adjustable width frame members configured to move the one or more adjustable width frame members move between a narrow configuration and a wide configuration.

Example 44. The system of example 43 wherein the one or more adjustable width frame members are attached directly to a distal portion of a main body of the device.

Example 45. The system of example 44 further comprising a spacer disposed over the main body of the device.

Example 46. The system of example 43 wherein the one or more adjustable width frame members are attached directly to a cap of the device.

Example 47. The system of example 46 wherein movement of the cap by the opening and closing member moves the one or more paddles between the open position and the closed position.

Example 48. The system of any one of examples 43-47 further comprising a connector coupled to the one or more adjustable width frame members configured to move the one or more adjustable width frame members between the narrow configuration and the wide configuration.

Example 49. The system of example 48 wherein the connector is pulled into a proximal portion of the device to narrow the one or more adjustable width frame members.

Example 50. The system of example 49 wherein the connector is pulled into a collar of the device.

Example 51. The system of example 48 wherein the connector includes attachment portions that connect to the one or more adjustable width frame members.

Example 52. The system of any one of examples 43-51 further comprising one or more inner frame members coupled to the one or more paddles configured to bias the one or more paddles to the closed position.

Example 52. The system of any one of examples 43-52 wherein each of the one or more adjustable width frame members have an anterior portion extending from the medial side of the device to the lateral side of the device and a posterior portion extending from the medial side of the device to the lateral side of the device opposite the anterior portion.

Example 54. The system of example 53, wherein the anterior portion and the posterior portion of each adjustable width frame member attach to one, or both, of an inner frame portion and the one or more paddles at a proximal portion of the device.

Example 55. The system of any one of examples 43-54, wherein each of the one or more adjustable width frame members comprise a shape-memory alloy.

Example 56. The system of any one of examples 43-55, wherein the one or more adjustable width frame members are symmetric.

Example 57. A method of repairing a native valve of a heart, comprising: moving one or more paddles of a device from a closed position to an open position; moving the one or more paddles from the open position to the closed position to attach the device to the native valve; adjusting a width of one or more adjustable width frame members by moving portions of the one or more adjustable width frame members through one or more guides; and wherein the one or more guides control a shape of the one or more adjustable width frame members as the one or more adjustable width frame members move between a narrow configuration and a wide configuration.

Example 58. The method of example 57 wherein each guide of the one or more guides comprises a sling.

Example 59. The method of example 57 wherein each guide of the one or more guides comprises a one or more loops.

Example 60. A method of repairing a native valve of a heart, comprising: moving one or more paddles of a device from a closed position to an open position; moving the one or more paddles from the open position to the closed position to attach the device to the native valve; and narrowing one or more adjustable width frame members by pulling the one or more adjustable width frame members proximally.

Example 61. The method of example 60 wherein the one or more adjustable width frame members are attached directly to a distal portion of a main body of the device.

Example 62. The method of example 60 wherein the one or more adjustable width frame members are pulled proximally by pulling a connector into a proximal portion of the device.

Example 63. The method of example 60 further comprising widening the one or more adjustable width frame members by pushing the one or more adjustable width frame members distally.

Example 64. The method of example 63 wherein the one or more adjustable width frame members are pushed distally by pushing a connector out of a proximal portion of the device.

Example 65. A device useable for treating and/or repairing a native valve of a heart, the device comprising: a central body having a proximal end portion, a distal end portion, and an inner space, a cap connected to the distal end portion of the central body; one or more paddles movable between an open position and a closed position, wherein the one or more paddles are configured to attach the device to the native valve; one or more adjustable width frame members coupled to the one or more paddles such that the one or more adjustable width frame members open and close with the one or more paddles; wherein the one or more adjustable width frame members comprise an outer frame portion extending from the distal end portion of the central body and extending from the inner space of the central body.

Example 66. The device of example 65, wherein the one or more adjustable width frame members further comprise an inner frame portion connecting to the outer frame portion and having a location in the inner space of the central body.

Example 67. The device of any one of examples 65-66, wherein the one or more adjustable width frame members comprise an expanded state having the outer frame portion in an extended position relative to the distal end portion of the central body.

Example 68. The device of any one of examples 65-67, wherein the one or more adjustable width frame members comprise a narrowed state having the outer frame member portion in a retracted position relative to the distal end portion of the central body.

Example 69. The device of any one of examples 65-68, wherein the outer frame portion of the one or more adjustable width frame members comprise an arcuate shape.

Example 70. The device of any one of examples 65-69, further comprising a connector extending through the inner space of the central body and connected to the one or more adjustable width frame members.

Example 71. The device of any one of examples 65-70, wherein the outer frame portion of the one or more adjustable width frame members is configured to extend from and retract into the distal end portion of the central body.

Example 72. The device of any one of examples 65-71, wherein the outer frame portion arcuately extends from the inner space of the central body and out from the distal end portion of the central body.

Example 73. The device of any one of examples 65-72, wherein the outer frame portion is configured to open the one or more adjustable width frame members when the outer frame portion is retracted into the distal end portion of the central body.

Example 74. The device of any one of examples 65-73, wherein the outer frame portion is configured to close the one or more adjustable width frame members when the outer frame portion is extended from the distal end portion of the central body.

Example 75. A device useable for treating and/or repairing a native valve of a heart, the device comprising: one or more paddles movable between an open position and a closed position, wherein the one or more paddles are configured to attach the device to the native valve; wherein the one or more paddles; one or more adjustable width frame members coupled to the one or more paddles such that the one or more adjustable width frame members open and close with the one or more paddles; wherein the one or more paddles comprises at least one guide portion and wherein portions of the one or more adjustable width frame members are positioned inside the at least one guide portion and can move within the at least one guide portion.

Example 76. The device of example 75, wherein the at least one guide portion comprises at least one cut-out.

Example 77. The device of any one of examples 75-76, wherein the at least one guide portion comprises at least one recess.

Example 78. The device of any one of examples 75-77, wherein the at least one guide portion comprises a closed shape having an inner open space.

Example 79. The device of any one of examples 75-78, wherein the at least one guide portion comprises at least one curved section.

Example 80. The device of any one of examples 75-79, wherein the at least one guide portion comprises at one projecting tab.

Example 81. The device of any one of examples 75-80, wherein the one or more paddles comprises a body portion having a distended shape section.

Example 82. The device of example 81, wherein the distended shape section comprises a first and second side portions, and wherein the first and second side portions each comprise the at least one guide.

Example 83. The device of any one of examples 81, wherein the body portion comprises first and second distended sections and the at least one guide between the first and second distended sections.

Example 84. A device useable for treating and/or repairing a native valve of a heart, the device comprising: one or more paddles movable between an open position and a closed position, wherein the one or more paddles are configured to attach the device to the native valve; one or more adjustable width frame members coupled to the one or more paddles such that the one or more adjustable width frame members open and close with the one or more paddles; wherein the one or more adjustable width frame members comprise a connection to the one or more paddles that comprises a pivot joint.

Example 85. The device of example 84, wherein the pivot joint comprises a hinge joint.

Example 86. The device of example 84, wherein the pivot joint comprises a rotational joint.

Example 87. The device of example 84, wherein the pivot joint comprises a flex joint.

Example 88. The device of any one of examples 84-87, wherein the one or more paddles comprise inner and outer paddle portions and the pivot joint comprises a location on the outer paddle portions.

Example 89. The device of any one of examples 84 and 86-88, wherein the pivot joint comprises a pin portion extending from the one or more paddles.

Example 90. The device of any one of examples 84 and 86-89, wherein the adjustable width frame members comprise a pin ring portion.

Example 91. The device of any one of examples 84-90, further comprising at least one activation connector connected to the adjustable width frame members.

Example 92. The device of any one of examples 84-91, wherein the one or more paddles comprise at least one guide portion for restricting movement of the adjustable width frame members.

Example 93. The device of any one of examples 84-92, wherein the adjustable width frame members comprise portions that rotate or flex about the pivot joint.

Example 94. The device of any one of examples 84-91 and 93, further comprising a guide portion connected to the one or more paddles, wherein the guide portion comprises a body having outer walls and an inner passageway for receiving portions of the adjustable width frame members.

Example 95. The device of any one of examples 84-94, wherein the adjustable width frame members comprise at least one strut portion connected to the pivot joint and at least one tab portion connected to the pivot joint and wherein movement of tab portion causes pivoting of the strut portion about the pivot joint.

Example 96. The device of any one of examples 84-94 wherein the adjustable width frame members comprise at least one strut portion connected to the pivot joint and at least one tab portion connected to the pivot joint and wherein movement of tab portion causes flexure of the strut portion relative to the pivot joint.

Example 97. The device of any one of examples 84-94 wherein the adjustable width frame members comprise first and second strut portions connected together and wherein the first strut portion is configured to pivot about the pivot joint when the second strut portion is moved.

Example 98. A device useable for treating and/or repairing a native valve of a heart, the device comprising: a central body having a proximal end portion and a distal end portion, a cap connected to the distal end portion of the central body and having a retaining portion; one or more paddles movable between an open position and a closed position, wherein the one or more paddles are configured to attach the device to the native valve; one or more adjustable width frame members coupled to the cap such that the one or more adjustable width frame members open and close with the one or more paddles; wherein the one or more adjustable width frame members comprise a connection to the cap that comprises a pivot joint.

Example 99. The device of example 98, wherein the pivot joint comprises a hinge joint.

Example 100. The device of example 98, wherein the pivot joint comprises a rotational joint.

Example 101. The device of example 98, wherein the pivot joint comprises a flex joint.

Example 102. The device of any one of examples 98-101, wherein the pivot joint comprises a pin portion extending from the one or more paddles.

Example 103. The device of any one of examples 98-102, wherein the adjustable width frame members comprise a pin ring portion.

Example 104. The device of any one of examples 98-100 and 102-104, wherein the cap comprises a retaining portion connected to the distal end of the base, spacer, center support member, receiver, or bushing and the pivot joint comprises a location on the retaining portion.

The techniques, methods, operations, steps, etc. described or suggested herein or in the references incorporated herein, and any methods of using the systems, assemblies, apparatuses, devices, etc. herein, can be performed on a living subject (e.g., human, other animal, etc.) or on a simulation (e.g., a cadaver, cadaver heart, simulator, imaginary person, etc.). When performed on a simulation, the body parts, e.g., heart, tissue, valve, etc., can be assumed to be simulated or can optionally be referred to as “simulated” (e.g., simulated heart, simulated tissue, simulated valve, etc.) and can optionally comprise computerized and/or physical representations of body parts, tissue, etc. The term “simulation” covers use on a cadaver, computer simulator, imaginary person (e.g., if they are just demonstrating in the air on an imaginary heart), etc.

Any of the various systems, assemblies, devices, components, apparatuses, etc. in this disclosure can be sterilized (e.g., with heat, radiation, ethylene oxide, hydrogen peroxide, etc.) to ensure they are safe for use with patients, and the methods herein can comprise (or additional methods comprise or consist of) sterilization of the associated system, device, component, apparatus, etc. (e.g., with heat, radiation, ethylene oxide, hydrogen peroxide, etc.).

While various inventive aspects, concepts and features of the disclosures can be described and illustrated herein as embodied in combination in the examples herein, these various aspects, concepts, and features can be used in many alternative examples, either individually or in various combinations and sub-combinations thereof. Unless expressly excluded herein all such combinations and sub-combinations are intended to be within the scope of the present application. Still further, while various alternative examples as to the various aspects, concepts, and features of the disclosures—such as alternative materials, structures, configurations, methods, devices, and components, alternatives as to form, fit, and function, and so on—may be described herein, such descriptions are not intended to be a complete or exhaustive list of available alternative examples, whether presently known or later developed. Those skilled in the art can readily adopt one or more of the inventive aspects, concepts, or features into additional examples and uses within the scope of the present application even if such examples are not expressly disclosed herein.

Additionally, even though some features, concepts, or aspects of the disclosures may be described herein as being a preferred arrangement or method, such description is not intended to suggest that such feature is required or necessary unless expressly so stated. Still further, example or representative values and ranges may be included to assist in understanding the present application, however, such values and ranges are not to be construed in a limiting sense and are intended to be critical values or ranges only if so expressly stated.

Moreover, while various aspects, features and concepts may be expressly identified herein as being inventive or forming part of a disclosure, such identification is not intended to be exclusive, but rather there may be inventive aspects, concepts, and features that are fully described herein without being expressly identified as such or as part of a specific disclosure, the disclosures instead being set forth in the appended claims. Descriptions of example methods or processes are not limited to inclusion of all steps as being required in all cases, nor is the order that the steps are presented to be construed as required or necessary unless expressly so stated. The words used in the claims have their full ordinary meanings and are not limited in any way by the description of the examples in the specification.

What is claimed is:

1. A device for treating and/or repairing a native valve of a heart, the device comprising:
one or more paddles movable between an open position and a closed position, wherein the one or more paddles are facilitate to attaching the device to the native valve;
one or more adjustable width frame members coupled to the one or more paddles such that the one or more adjustable width frame members open and close with the one or more paddles;
one or more guides that comprise one or more loops, slings or rings;
wherein the one or more adjustable width frame members extend through the one or more guides; and
wherein the one or more guides pinch portions of the adjustable width frame members to control a shape of the one or more adjustable width frame members as the one or more adjustable width frame members move between a wide configuration and a narrow configuration.

2. The device of claim 1, further comprising a connector coupled to the one or more adjustable width frame members configured to move the one or more adjustable width frame members between the narrow configuration and the wide configuration.

3. The device of claim 2, wherein the connector includes attachment portions that connect to the one or more adjustable width frame members.

4. The device of claim 2, wherein the one or more adjustable width frame members are attached to a distal portion of the device and the connector is pulled into a proximal portion of the device to narrow the one or more adjustable width frame members.

5. The device of claim 1, further comprising one or more inner frame members coupled to the one or more paddles configured to bias the one or more paddles to the closed position.

6. The device of claim 1, wherein the one or more adjustable width frame members are attached to a proximal portion of the device.

7. A device useable for treating and/or repairing a native valve of a heart, the device comprising:
one or more paddles movable between an open position and a closed position, wherein the one or more paddles are configured to attach the device to the native valve;
one or more adjustable width frame members coupled to the one or more paddles such that the one or more adjustable width frame members open and close with the one or more paddles;
a connector coupled to the one or more adjustable width frame members configured to move the one or more adjustable width frame members between a narrow configuration and a wide configuration;
wherein the one or more adjustable width frame members are attached to a distal portion of the device and the connector is pulled into a proximal portion of the device to narrow the one or more adjustable width frame members.

8. The device of claim 7, wherein the connector includes attachment portions that connect to the one or more adjustable width frame members.

9. The device of claim 7, further comprising one or more inner frame members coupled to the one or more paddles configured to bias the one or more paddles to the closed position.

10. The device of claim 7, wherein each of the one or more adjustable width frame members have an anterior portion extending from the medial side of the device to the lateral side of the device and a posterior portion extending from the medial side of the device to the lateral side of the device opposite the anterior portion.

11. The device of claim 10, wherein the anterior portion and the posterior portion of each adjustable width frame member attach to one, or both, of an inner frame portion and the one or more paddles at a proximal portion of the device.

12. A device useable for treating and/or repairing a native valve of a heart, the device comprising:
one or more paddles movable between an open position and a closed position, wherein the one or more paddles are configured to attach the device to the native valve;
one or more adjustable width frame members coupled to the one or more paddles such that the one or more adjustable width frame members open and close with the one or more paddles; and
wherein the one or more adjustable width frame members comprise a connection to the one or more paddles that comprises a pivot joint.

13. The device of claim 12 wherein the pivot joint comprises a hinge joint, a rotational joint, or a flex joint.

14. The device of claim 12, wherein the one or more paddles comprise inner and outer paddle portions and the pivot joint comprises a location on the outer paddle portions.

15. The device of claim 12, wherein the pivot joint comprises a pin portion extending from the one or more paddles.

16. The device of claim 12, wherein the adjustable width frame members comprise a pin ring portion.

17. The device of claim 12, further comprising at least one activation connector connected to the adjustable width frame members.

18. The device of claim 12, wherein the one or more paddles comprise at least one guide portion for restricting movement of the adjustable width frame members.

19. The device of claim 12, wherein the adjustable width frame members comprise portions that rotate or flex about the pivot joint.

20. The device of claim 12, further comprising a guide portion connected to the one or more paddles, wherein the guide portion comprises a body having outer walls and an inner passageway for receiving portions of the adjustable width frame members.
